SEQUENCE LISTING (36 sequences)

SEQ ID NO: 1 - DNA, 20 bp, synthetic construct
  source 1..20: mol_type = other DNA
cagaactcaagataggcagc

SEQ ID NO: 2 - DNA, 20 bp, synthetic construct
  source 1..20: mol_type = other DNA
gtgaaattcctgatgcggtc

SEQ ID NO: 3 - DNA, 20 bp, synthetic construct
  source 1..20: mol_type = other DNA
gaagtagcactgtacctcct

SEQ ID NO: 4 - DNA, 20 bp, synthetic construct
  source 1..20: mol_type = other DNA
acagagtcaggacaaatggc

SEQ ID NO: 5 - DNA, 20 bp, synthetic construct
  source 1..20: mol_type = other DNA
cactcttgcagcagacattc

SEQ ID NO: 6 - DNA, 20 bp, synthetic construct
  source 1..20: mol_type = other DNA
agaatcaaggactctcggag

SEQ ID NO: 7 - DNA, 20 bp, synthetic construct
  source 1..20: mol_type = other DNA
aggtaaactggtgagggcaa

SEQ ID NO: 8 - DNA, 20 bp, synthetic construct
  source 1..20: mol_type = other DNA
aaggtacttcatgccagagg

SEQ ID NO: 9 - DNA, 20 bp, synthetic construct
  source 1..20: mol_type = other DNA
ctgctcattccaaagtcagc

SEQ ID NO: 10 - DNA, 20 bp, synthetic construct
  source 1..20: mol_type = other DNA
gcactgacatctagggcaaa

SEQ ID NO: 11 - DNA, 19 bp, synthetic construct
  source 1..19: mol_type = other DNA
tattgttgaacatgctaga

SEQ ID NO: 12 - DNA, 19 bp, synthetic construct
  source 1..19: mol_type = other DNA
tcgtatctcttctccagca

SEQ ID NO: 13 - DNA, 19 bp, synthetic construct
  source 1..19: mol_type = other DNA
tcttcgtatctcttctcca

SEQ ID NO: 14 - DNA, 19 bp, synthetic construct
  source 1..19: mol_type = other DNA
aattgatcttgtacatggg

SEQ ID NO: 15 - DNA, 19 bp, synthetic construct
  source 1..19: mol_type = other DNA
tgctgtagtgaacttgccc

SEQ ID NO: 16 - DNA, 19 bp, synthetic construct
  source 1..19: mol_type = other DNA
tcatggtagtgaaattcct

SEQ ID NO: 17 - DNA, 20 bp, synthetic construct
  source 1..20: mol_type = other DNA
tctccagagcaccatactga

SEQ ID NO: 18 - DNA, 20 bp, synthetic construct
  source 1..20: mol_type = other DNA
cctccgtgtttgatcctgat

SEQ ID NO: 19 - DNA, 20 bp, synthetic construct
  source 1..20: mol_type = other DNA
cacttgatgttcatgcggga

SEQ ID NO: 20 - DNA, 20 bp, synthetic construct
  source 1..20: mol_type = other DNA
gcaagccagtctacatcaag

SEQ ID NO: 21 - DNA, 20 bp, synthetic construct
  source 1..20: mol_type = other DNA
ccctcagacacccacaatta

SEQ ID NO: 22 - DNA, 20 bp, synthetic construct
  source 1..20: mol_type = other DNA
cgtaccgtgatcagaaagtg

SEQ ID NO: 23 - DNA, 20 bp, synthetic construct
  source 1..20: mol_type = other DNA
ccctgtctcttgaagtttgc

SEQ ID NO: 24 - DNA, 20 bp, synthetic construct
  source 1..20: mol_type = other DNA
tctctgtgctgtcgttgtct

SEQ ID NO: 25 - DNA, 20 bp, synthetic construct
  source 1..20: mol_type = other DNA
atccatccatccatcctgct

SEQ ID NO: 26 - DNA, 20 bp, synthetic construct
  source 1..20: mol_type = other DNA
cgtcctgtcttgctttgtct

SEQ ID NO: 27 - DNA, 20 bp, synthetic construct
  source 1..20: mol_type = other DNA
ggactacgaattcacaaggc

SEQ ID NO: 28 - DNA, 20 bp, synthetic construct
  source 1..20: mol_type = other DNA
acattgttggaaagggaggc

SEQ ID NO: 29 - DNA, 20 bp, synthetic construct
  source 1..20: mol_type = other DNA
aacaaaccaggtcctttggc

SEQ ID NO: 30 - DNA, 20 bp, synthetic construct
  source 1..20: mol_type = other DNA
gcaatggtggtcagtgtctg

SEQ ID NO: 31 - DNA, 20 bp, synthetic construct
  source 1..20: mol_type = other DNA
ggttcagaatgagtcagtgg

SEQ ID NO: 32 - DNA, 20 bp, synthetic construct
  source 1..20: mol_type = other DNA
tctctggaggtaaggcacat

SEQ ID NO: 33 - DNA, 20 bp, synthetic construct
  source 1..20: mol_type = other DNA
tctgaaacacggtctggatt

SEQ ID NO: 34 - DNA, 20 bp, synthetic construct
  source 1..20: mol_type = other DNA
atgccaggttgctcagattc

SEQ ID NO: 35 - DNA, 20 bp, synthetic construct
  source 1..20: mol_type = other DNA
agtttgacagtatctgccca

SEQ ID NO: 36 - DNA, 20 bp, synthetic construct
  source 1..20: mol_type = other DNA
acacgctccacctttattcc

(19) United States
(12) Patent Application Publication
(10) Pub. No.: US 2026/0284095 A1
(43) Pub. Date: Sep. 24, 2026

(54) METHODS AND MATERIALS FOR TREATING PROGRESSIVE SUPRANUCLEAR PALSY

(71) Applicant: Mayo Foundation for Medical Education and Research, Rochester, MN (US)

(72) Inventors: Mariet A. Younkin, Jacksonville Beach, FL (US); Yuhao Min, Jacksonville, FL (US); Nilufer Taner, Jacksonville, FL (US); Xue Wang, Jacksonville, FL (US)

(21) Appl. No.: 19/479,794

(22) PCT Filed: May 1, 2024
(86) PCT No.: PCT/US2024/027237

§ 371 Date: Oct. 29, 2025

Related U.S. Application Data
(60) Provisional application No. 63/463,212, filed on May 1, 2023.

(51) Int. Cl. A61K 31/7088; A61K 9/00; A61P 25/28

(52) U.S. Cl. CPC A61K 31/7088; A61K 9/0019; A61P 25/28

(57) ABSTRACT

This document relates to methods and materials for treating progressive supranuclear palsy (PSP) in a mammal (e.g., a human). For example, methods and materials for using one or more inhibitors of a glial polypeptide to treat a mammal having PSP are provided.

### CROSS-REFERENCE TO RELATED APPLICATIONS

This application claims the benefit of U.S. Patent Application Ser. No. 63/463,212, filed on May 1, 2023. The disclosure of the prior application is considered part of, and is incorporated by reference in, the disclosure of this application.

### STATEMENT REGARDING FEDERAL FUNDING

This invention was made with government support under AG046139, AG061796, and NS080820 awarded by the National Institutes of Health. The government has certain rights in the invention.

### SEQUENCE LISTING

This application contains a Sequence Listing that has been submitted electronically as an XML file named “07039-2185WO_SL.xml.” The XML file, created on Apr. 13, 2024, is 32000 bytes in size. The material in the XML file is hereby incorporated by reference in its entirety.

### TECHNICAL FIELD

This document relates to methods and materials for treating progressive supranuclear palsy (PSP) in a mammal (e.g., a human). For example, this document provides methods and materials for using one or more inhibitors of a glial polypeptide to treat a mammal having PSP.

### BACKGROUND INFORMATION

PSP, also referred to as Steele-Richardson-Olszewski syndrome, is a neurodegenerative disorder with an early age of onset and rapid progression to death (Coyle-Gilchrist et al., Neurology 86:1736-1743 (2016)). PSP is a primary tauopathy characterized by the overexpression of 4-repeat tau polypeptide isoform in both neuronal and glial cells, leading to cell-specific tau lesions including neurofibrillary tangles (NFT) in the neurons, tufted astrocytes (TA), coiled bodies (CB) in oligodendrocytes, and tau threads (TauTh) in white matter.

The disease mechanism(s) and key molecular player(s) that underlie PSP pathogenesis remain to be elucidated, creating a barrier to develop treatments.

### SUMMARY

This document provides methods and materials for treating PSP. For example, this document provides methods and materials for administering one or more inhibitors of a glial polypeptide to a mammal (e.g., a human) having PSP to treat the mammal. As described herein, glial polypeptides such as discoidin domain receptor tyrosine kinase 2 (DDR2) polypeptides, KN motif and ankyrin repeat domains 2 (KANK2) polypeptides, and stomatin (STOM) polypeptides can be upregulated in PSP. Also as described herein, one or more inhibitors of a glial polypeptide can be used treat a mammal (e.g., a human) having PSP. For example, one or more inhibitors of a glial polypeptide can be administered to a mammal (e.g., a human) having PSP to treat the mammal. In some cases, one or more inhibitors of a glial polypeptide can be used to reduce or slow neurodegeneration (e.g., tau-related neurodegeneration) within the brain of a mammal (e.g., a human) having PSP and/or to reduce a level of tau polypeptides (e.g., 4-repeat tau polypeptides) in the brain of a mammal (e.g., a human) having PSP.

In general, one aspect of this document features methods for treating a mammal having PSP, wherein the methods can include, or consist essentially of, administering an inhibitor of a glial polypeptide to said mammal. The mammal can be a human. The method can include identifying said mammal as having said PSP prior to said administering. The inhibitor of a glial polypeptide can inhibit a DDR2 polypeptide, a KANK2 polypeptide, or a STOM polypeptide. The inhibitor of said glial polypeptide can be an inhibitor of DDR2 polypeptide activity. The inhibitor of said DDR2 polypeptide activity can be an anti-DDR2 antibody. The inhibitor of said glial polypeptide can be an inhibitor of DDR2 polypeptide expression. The inhibitor of said DDR2 polypeptide expression can be a nucleic acid molecule designed to induce RNA interference of said DDR2 polypeptide expression. The nucleic acid molecule designed to induce RNA interference of said DDR2 polypeptide expression can comprise, consist essentially or, or consist of a nucleic acid sequence set forth in any one of SEQ ID NOs: 1-16. The inhibitor of said glial polypeptide can be an inhibitor of KANK2 polypeptide activity. The inhibitor of said KANK2 polypeptide activity can be an anti-KANK2 antibody. The inhibitor of said glial polypeptide can be an inhibitor of KANK2 polypeptide expression. The inhibitor of said KANK2 polypeptide expression can be a nucleic acid molecule designed to induce RNA interference of said KANK2 polypeptide expression. The nucleic acid molecule designed to induce RNA interference of said KANK2 polypeptide expression can comprise, consist essentially or, or consist of a nucleic acid sequence set forth in any one of SEQ ID NOs: 17-26. The inhibitor of said glial polypeptide can be an inhibitor of STOM polypeptide activity. The inhibitor of said STOM polypeptide activity can be an anti-STOM antibody. The inhibitor of said glial polypeptide can be an inhibitor of STOM polypeptide expression. The inhibitor of said STOM polypeptide expression can be a nucleic acid molecule designed to induce RNA interference of said STOM polypeptide expression. The nucleic acid molecule designed to induce RNA interference of said STOM polypeptide expression can comprise, consist essentially or, or consist of a nucleic acid sequence set forth in any one of SEQ ID NOs: 27-36.

In another aspect, this document features uses of a composition comprising an inhibitor of a glial polypeptide to treat a mammal having PSP. The inhibitor of a glial polypeptide can inhibit a DDR2 polypeptide, a KANK2 polypeptide, or a STOM polypeptide. The inhibitor of said glial polypeptide can be an inhibitor of DDR2 polypeptide activity. The inhibitor of said DDR2 polypeptide activity can be an anti-DDR2 antibody. The inhibitor of said glial polypeptide can be an inhibitor of DDR2 polypeptide expression. The inhibitor of said DDR2 polypeptide expression can be a nucleic acid molecule designed to induce RNA interference of said DDR2 polypeptide expression. The nucleic acid molecule designed to induce RNA interference of said DDR2 polypeptide expression can comprise, consist essentially or, or consist of a nucleic acid sequence set forth in any one of SEQ ID NOs: 1-16. The inhibitor of said glial polypeptide can be an inhibitor of KANK2 polypeptide activity. The inhibitor of said KANK2 polypeptide activity can be an anti-KANK2 antibody. The inhibitor of said glial polypeptide can be an inhibitor of KANK2 polypeptide expression. The inhibitor of said KANK2 polypeptide expression can be a nucleic acid molecule designed to induce RNA interference of said KANK2 polypeptide expression. The nucleic acid molecule designed to induce RNA interference of said KANK2 polypeptide expression can comprise, consist essentially or, or consist of a nucleic acid sequence set forth in any one of SEQ ID NOs: 17-26. The inhibitor of said glial polypeptide can be an inhibitor of STOM polypeptide activity. The inhibitor of said STOM polypeptide activity can be an anti-STOM antibody. The inhibitor of said glial polypeptide can be an inhibitor of STOM polypeptide expression. The inhibitor of said STOM polypeptide expression can be a nucleic acid molecule designed to induce RNA interference of said STOM polypeptide expression. The nucleic acid molecule designed to induce RNA interference of said STOM polypeptide expression can comprise, consist essentially or, or consist of a nucleic acid sequence set forth in any one of SEQ ID NOs: 27-36. The mammal can be a human.

In another aspect, this document features compositions comprising an inhibitor of a glial polypeptide for use in the preparation of a medicament to treat a mammal having PSP. The inhibitor of a glial polypeptide can inhibit a DDR2 polypeptide, a KANK2 polypeptide, or a STOM polypeptide. The inhibitor of said glial polypeptide can be an inhibitor of DDR2 polypeptide activity. The inhibitor of said DDR2 polypeptide activity can be an anti-DDR2 antibody. The inhibitor of said glial polypeptide can be an inhibitor of DDR2 polypeptide expression. The inhibitor of said DDR2 polypeptide expression can be a nucleic acid molecule designed to induce RNA interference of said DDR2 polypeptide expression. The nucleic acid molecule designed to induce RNA interference of said DDR2 polypeptide expression can comprise, consist essentially or, or consist of a nucleic acid sequence set forth in any one of SEQ ID NOs: 1-16. The inhibitor of said glial polypeptide can be an inhibitor of KANK2 polypeptide activity. The inhibitor of said KANK2 polypeptide activity can be an anti-KANK2 antibody. The inhibitor of said glial polypeptide can be an inhibitor of KANK2 polypeptide expression. The inhibitor of said KANK2 polypeptide expression can be a nucleic acid molecule designed to induce RNA interference of said KANK2 polypeptide expression. The nucleic acid molecule designed to induce RNA interference of said KANK2 polypeptide expression can comprise, consist essentially or, or consist of a nucleic acid sequence set forth in any one of SEQ ID NOs: 17-26. The inhibitor of said glial polypeptide can be an inhibitor of STOM polypeptide activity. The inhibitor of said STOM polypeptide activity can be an anti-STOM antibody. The inhibitor of said glial polypeptide can be an inhibitor of STOM polypeptide expression. The inhibitor of said STOM polypeptide expression can be a nucleic acid molecule designed to induce RNA interference of said STOM polypeptide expression. The nucleic acid molecule designed to induce RNA interference of said STOM polypeptide expression can comprise, consist essentially or, or consist of a nucleic acid sequence set forth in any one of SEQ ID NOs: 27-36. The mammal can be a human.

In another aspect, this document features compositions comprising an inhibitor of a glial polypeptide for use in the treatment of a mammal having PSP. The inhibitor of a glial polypeptide can inhibit a DDR2 polypeptide, a KANK2 polypeptide, or a STOM polypeptide. The inhibitor of said glial polypeptide can be an inhibitor of DDR2 polypeptide activity. The inhibitor of said DDR2 polypeptide activity can be an anti-DDR2 antibody. The inhibitor of said glial polypeptide can be an inhibitor of DDR2 polypeptide expression. The inhibitor of said DDR2 polypeptide expression can be a nucleic acid molecule designed to induce RNA interference of said DDR2 polypeptide expression. The nucleic acid molecule designed to induce RNA interference of said DDR2 polypeptide expression can comprise, consist essentially or, or consist of a nucleic acid sequence set forth in any one of SEQ ID NOs: 1-16. The inhibitor of said glial polypeptide can be an inhibitor of KANK2 polypeptide activity. The inhibitor of said KANK2 polypeptide activity can be an anti-KANK2 antibody. The inhibitor of said glial polypeptide can be an inhibitor of KANK2 polypeptide expression. The inhibitor of said KANK2 polypeptide expression can be a nucleic acid molecule designed to induce RNA interference of said KANK2 polypeptide expression. The nucleic acid molecule designed to induce RNA interference of said KANK2 polypeptide expression can comprise, consist essentially or, or consist of a nucleic acid sequence set forth in any one of SEQ ID NOs: 17-26. The inhibitor of said glial polypeptide can be an inhibitor of STOM polypeptide activity. The inhibitor of said STOM polypeptide activity can be an anti-STOM antibody. The inhibitor of said glial polypeptide can be an inhibitor of STOM polypeptide expression. The inhibitor of said STOM polypeptide expression can be a nucleic acid molecule designed to induce RNA interference of said STOM polypeptide expression. The nucleic acid molecule designed to induce RNA interference of said STOM polypeptide expression can comprise, consist essentially or, or consist of a nucleic acid sequence set forth in any one of SEQ ID NOs: 27-36. The mammal can be a human.

In another aspect, this document features methods for treating a mammal having PSP, wherein said method can include, or consist essentially of, administering an inhibitor of a KANK2 polypeptide to said mammal. The inhibitor of said KANK2 polypeptide can be an anti-KANK2 antibody. The inhibitor of said KANK2 polypeptide can be a nucleic acid molecule designed to induce RNA interference of said KANK2 polypeptide expression. The nucleic acid molecule designed to induce RNA interference of said KANK2 polypeptide expression can comprise, consist essentially or, or consist of a nucleic acid sequence set forth in any one of SEQ ID NOs: 17-26. The administering can also include administering an inhibitor of a DDR2 polypeptide. The inhibitor of said DDR2 polypeptide can be an anti-DDR2 antibody. The inhibitor of said DDR2 polypeptide can be a nucleic acid molecule designed to induce RNA interference of said DDR2 polypeptide expression. The nucleic acid molecule designed to induce RNA interference of said DDR2 polypeptide expression can comprise, consist essentially or, or consist of a nucleic acid sequence set forth in any one of SEQ ID NOs: 1-16. The administering can also include administering an inhibitor of a STOM polypeptide. The inhibitor of said STOM polypeptide can be an anti-STOM antibody. The inhibitor of said STOM polypeptide can be a nucleic acid molecule designed to induce RNA interference of said STOM polypeptide expression. The nucleic acid molecule designed to induce RNA interference of said STOM polypeptide expression can comprise, consist essentially or, or consist of a nucleic acid sequence set forth in any one of SEQ ID NOs: 27-36. The administering can include administering an inhibitor of a DDR2 polypeptide and administering an inhibitor of a STOM polypeptide. The inhibitor of said DDR2 polypeptide can be an anti-DDR2 antibody. The inhibitor of said DDR2 polypeptide can be a nucleic acid molecule designed to induce RNA interference of said DDR2 polypeptide expression. The nucleic acid molecule designed to induce RNA interference of said DDR2 polypeptide expression can comprise, consist essentially or, or consist of a nucleic acid sequence set forth in any one of SEQ ID NOs: 1-16. The inhibitor of said STOM polypeptide can be an anti-STOM antibody. The inhibitor of said STOM polypeptide can be a nucleic acid molecule designed to induce RNA interference of said STOM polypeptide expression. The nucleic acid molecule designed to induce RNA interference of said STOM polypeptide expression can comprise, consist essentially or, or consist of a nucleic acid sequence set forth in any one of SEQ ID NOs: 27-36.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1E show that bulk RNAseq analysis identified vast and replicable differentially expressed genes (DEGs) in PSP brains. FIG. 1A is a table showing the number of DEGs identified with respect to different phenotypes at different thresholds. FIG. 1B is a heatmap of log FC (for PSPvsCtrl) and association beta (for neuropathology) of the top genes. Top genes were defined as lowest 5% false discovery rate (FDR) and top 5% of the absolute value of the log FC. Top genes that are also marker genes for astrocyte, endothelia, and oligodendrocyte are shown. FIG. 1C is a volcano plot for all DEGs with FDR<0.05 comparing gene expression levels between PSP and control. FIG. 1D is a series of graphs showing the distribution of the expression levels of MOBP, KANSL1-AS1, YAP1, and CLU between PSP and control in each study cohort. Statistical test: differential expression analysis using linear regression adjusting for covariates. Significance: FDR-adjusted p values (*=p<0.05, 0.01 **, 0.001 ***, 1×10⁻⁴ ****). FIG. 1E is a heatmap for the 4 genes highlighted in FIG. 1D.

FIGS. 2A-2D show that glial cell-enriched gene co-expression network modules are associated with PSP. FIG. 2A shows the associations between the module eigengenes (ME) and PSP diagnosis or quantitative tau neuropathology that were assessed. FIG. 2B shows enrichment of cell type specific genes that were detected in seven modules, three of which (M3-oligodendrocyte, M4=astrocyte, and M6-microglia/endothelia) were significantly associated with PSP. FIG. 2C is a series of plots showing that the associations were robust and consistent across both studies and were significant in the consensus network modules M3 (oligodendrocyte-enriched), M4 (astrocyte-enriched), and M6 (microglial/endothelia-enriched). Statistical test: t-test comparing PSP and control module eigengenes. Significance: Bonferroni-adjusted p values (*=p<0.05, 0.01 **, 0.001 ***, 1×10⁻⁴ ****). FIG. 2D shows the top enriched gene ontology biological process terms in the PSP-associated glial cell-enriched modules.

FIGS. 3A-3E show that single-nucleus RNAseq captured glial expression changes in PSP brains. FIG. 3A is a graph showing the nuclei in UMAP space by cluster number and grouped by cell type. UMAP is a dimension reduction technique and has been described elsewhere (see, for example, McInnes. ArXiv e-prints 1802.03426, 2018). FIG. 3B shows the average expression and percent nuclei expressed for each cell type marker gene used for cluster type assignment in each single nucleus RNA sequencing (snRNAseq) cluster. FIG. 3C is a graph of the count and proportion of each nuclei type. FIG. 3D is a series of graphs showing expression levels of module genes from the three cell-type-enriched, PSP-associated expression modules (M3, M4, M6) in the snRNAseq clusters for each cell type. FIG. 3E shows the number of overlapping genes between snRNAseq DEGs in each cluster and the three modules. P value: Fisher's exact test for the enrichment of snRNAseq DEGs in the expression module. Pie chart demonstrates the proportion of up- and down-regulated genes. The radii of the pie charts reflect the significance of the overlap, where significant overlaps (p<0.05) have solid outline. Transparency of pie chart reflects the size of the overlap between module genes and cluster DEGs (more opaque=higher number of overlapping genes).

FIGS. 4A-4F show an exemplary prioritization and experimental validation strategy for PSP brain transcriptome changes. FIG. 4A is an exemplary schematic prioritization approach using bulk brain human RNAseq, snRNAseq, and rTG4510 tau mouse brain transcriptome data, which led to the identification of 21 high confidence glial perturbed genes in PSP. Of these, 11 had Drosophila ortholog genes with RNAi available for in vivo validation. FIG. 4B is an upset plot showing the overlap of different validation methods and the number of genes selected at each filtering stage from each module. FIG. 4C is a plot of the number of genes identified in the Drosophila screen. FIG. 4D shows representative images of Drosophila eyes taken during the screening that indicated a strong rescue effect when inhibiting the expression of DDR2 or STOM. FIG. 4E is series of graphs showing expression levels of the top in vivo validated genes in bulk RNAseq dataset. Horizontal bars indicate median expression levels. Statistical test: differential expression analysis using linear regression adjusting for covariates. Significance: FDR-adjusted p values (*=p<0.05, 0.01 **, 0.001 ***). FIG. 4F shows expression levels of the top in vivo validated genes in the snRNAseq dataset. Statistical test: Zero-inflated regression adjusting for sex and age, implemented by the MAST package. Significance: FDR-adjusted (FDR) or unadjusted (P) p values as indicated.

FIG. 5 is a circos plot showing genes from the three glial cell-enriched, PSP associated gene expression modules. Modules M3, M4, and M6 (outer-most=first ring) and their bulk DEG directions of PSP associations (second ring), module membership (third ring, module membership scores are color-coded), validation in snRNAseq (fourth ring), tau mouse model rTG4510 brain RNAseq (fifth ring), and Drosophila tau model RNAi in vivo validations (sixth ring, Drosophila eye neurodegeneration morphology scores are color-coded) are shown. * indicates the 11 high confidence PSP glial perturbed hub genes that have available Drosophila orthologs and were validated experimentally in vivo. Refer to FIG. 4A-4B for numbers of genes filtered at each stage of the prioritization approach.

FIG. 6 shows the distribution of the median R package CQN value for each gene in the two bulk RNAseq cohorts. Based on the bimodal distribution, expression thresholds were chosen at 1 for cohort 1 and −2 for cohort 2 (dotted lines).

FIG. 7 is a series of graphs showing the correlation between beta coefficients from PSP vs control DEG and beta coefficients from gene expression associations with quantitative tau neuropathology in PSP.

FIGS. 8A-8B are heatmaps of bulk DEGs between PSP and control brains that represent the lowest 5% FDR (statistically significant; FIG. 8A) or the highest absolute value of the log of the fold chance (log FC), which is the largest expression difference; FIG. 8B).

FIG. 9 is a volcano plot showing DEGs between PSP and control that are within 1 Mbp of genes previously identified as PSP genetic risk factors.

FIGS. 10A-10B show that the consensus weighted gene co-expression network analysis (WGCNA network) definition is robust. FIG. 10A shows independent embedding of the expression data in T-distributed Stochastic Neighbor Embedding (t-SNE) space showing high agreement between the gene cluster and module assignment. FIG. 10B shows module preservation analysis and indicates that all modules were well-preserved with a summary score above the threshold of 10 (dotted lines).

FIG. 11 is a series of graphs showing the module eigengene distribution between PSP and control samples in study 1, study 2, and the consensus network for all 16 modules.

FIG. 12 is a series of graphs showing the top enriched Gene Ontology biological process terms in each of the WGCNA modules.

FIGS. 13A-13C are stacked bar plots showing the proportion of nuclei from different (FIG. 13A) sex, (FIG. 13B) diagnosis, and (FIG. 13C) sample in each snRNAseq cluster. There was no statistically significant enrichment of nuclei from either sex or diagnosis in any of the clusters. Eight clusters had statistically significant enrichment of nuclei from a small number of samples, depicted with sample numbers on the clusters. All but one of these 8 clusters (CL12) constitute<1% of total nuclei.

FIG. 14 is a graph showing the expression of cell-type marker genes in each snRNAseq cluster and confirming the identified clusters.

FIG. 15 is a table showing that enrichment of cell-type marker genes in each cluster identified using the R package ‘BRETIGEA’ corresponds to the cluster type. BRETIGEA cell-types for each column are shown at the bottom and number of BRETIGEA genes for these cell-types expressed in the snRNAseq data is shown at the top. P value and fold enrichment of these BRETIGEA cell-type marker genes for each cluster is shown in the data cells.

FIG. 16 shows overlap of WGCNA module genes and snRNASeq DEG genes. The number of overlapping genes between snRNAseq DEGs in each cluster and the 16 WGCNA modules are shown. P value: Fisher's exact test for the enrichment of snRNAseq DEGs in the expression module. Pie chart demonstrates proportion of up- and down-regulated genes. The radii of the pie charts reflect the significance of the overlap, where significant overlaps (p<0.05) have solid outline. Transparency of pie chart reflects the size of the overlap between module genes and cluster DEGs (more opaque-higher number of overlapping genes).

FIG. 17 is a series of graphs showing the expression level of the Drosophila tau model screened genes in PSP and control in bulk RNAseq.

FIG. 18 is a series of graphs showing the expression levels of the Drosophila tau model screened genes in PSP and control in snRNAseq.

FIG. 19 is a heat map of the bulk RNAseq log fold change of the 155 sn-validated genes.

FIG. 20 contains graphs showing principal component analysis (PCA) of the mouse expression data. No outlier was detected. Genotype+=rTg4510 mice; −=non-transgenic littermates.

FIG. 21 contains graphs showing the expression of chromosome Y genes RPS4Y1, DIF1AY, DDX3Y, and KDMD5D as a sex check for the mouse sample. Genotype: +=rTg4510 mice; −=non-transgenic littermates.

FIG. 22 is a schematic of the workflow used to identify dysregulated genes in the brain underlying PSP risk and pathogenesis.

FIGS. 23A-23B show results of RNAi knockdown of STOM, KANK2, and DDR2 in Drosophila. FIG. 23A) Representative Drosophila eye pictures indicate a robust rescue of tau-mediated toxicity when inhibiting the expression of the fly ortholog of STOM, KANK2, or DDR2 with RNAi. FIG. 23B) Statistical tests (two-sided Wilcoxon rank sum tests) showed a significant reduction in eye degeneration (n=10 flies per genotype were assessed).

FIG. 24 is a graph showing that treatment of Drosophila eyes with the DDR2 inhibitor dasatinib created a dosage-dependent reduction in tau-mediated eye degeneration.

FIG. 25 contains a gene expression analysis showing that DDR2, KANK2, and STOM were up-regulated in the transgenic mice expressing human P301S mutant Tau compared to non-transgenic control.

FIG. 26 contains a graph showing that knocking down the drosophila orthologs DDR2, KANK2, and STOM reduced tau seeding activity in tau transgenic Drosophila brains.

FIG. 27 contains a gene expression analysis showing that DDR2, KANK2, and STOM had similar expression levels in H4 neuroglioma cells (ATCC cat #HTB-148) cells and in PSP brains, indicating H4 cell line is suitable for conducting ASO screening experiments

FIG. 28 contains graphs of cycle thresholds (Ct values) showing that cDNA synthesis kits (High-cap and VILO) quantitatively reverse transcribed RNA into cDNA. The target gene quantity correlates to the RNA input for all genes in the study, validating the cDNA synthesis kit.

FIG. 29 contains graphs of Ct values showing that qPCR master mixes (Fast Advance, Universal) quantitatively amplified cDNA to generate signals. The target gene quantity correlates to the RNA input for all genes in the study, validating the qPCR mix.

FIG. 30 contains graphs of Ct values showing that duplex qPCR with probes targeting housekeeping genes and genes of interest yielded similar results as single qPCR reaction, validating the duplex qPCR setup.

FIG. 31 contains a gene expression analysis when a positive control antisense oligonucleotide (ASO) targeting PPIB was delivered to H4 cells. The transfection method (forward transfection vs reverse transfection) yielded similar results.

FIGS. 32A-32F contain graphs showing gene knockdown efficiency (FIGS. 32A, 32C, and 32E) and percent toxicity measured by LDH assay (FIGS. 32B, 32D, and 32F) following administration of ASOs targeting DDR2 (FIGS. 32A and 32B), KANK2 (FIGS. 32C and 32D), or STOM (FIGS. 32E and 32F).

FIGS. 33A-33C contain graphs showing gene knockdown efficiency (FIG. 33A), percent toxicity measured by LDH assay (FIG. 33B), and percent viability measured by XTT assay (FIG. 33C) following administration of ASOs targeting DDR2, KANK2, or STOM for the ASO hits (D-4, D-4, D-9, K-4, K-5, K10, S-1, S-4, and S-10).

FIG. 34 contains dose-response curves for various concentrations of ASOs in H4 cells. The half-maximal inhibitory concentration (IC50) for each ASO hit (D-4, D-4, D-9, K-4, K-5, K10, S-1, S-4, and S-10) was calculated with a four-parameter log-logistic model.

FIG. 35 contains a representative western blot image (top) and a graph quantifying the western blot (bottom) showing the protein levels of a DDR2 polypeptide following administration of ASOs targeting DDR2 in H4 cells. Statistics: two-sided t-tests comparing the beta-actin normalized DDR2 band intensity between DDR2-ASO treated against non-targeting-ASO (NT) treated cells. (p: *<0.05, **<0.01, ***<0.001, ****<1E-4).

FIGS. 36A-36B contain immunohistochemistry images showing that iPSCs expressed pluripotency markers (FIG. 36A) and showing that iPSCs differentiated into three germ layers (FIG. 36B), proving their pluripotency.

FIG. 37 contains immunohistochemistry images showing that iPSCs differentiated into neuronal progenitor cells (NPCs, Nestin), neurons (beta III tubulin+), and astrocytes (S100 beta+).

FIG. 38 shows the ASOs treatment scheme for neurons and shows that ASO D5 modulated DDR2 mRNA in iPSC-derived neurons in a dose-dependent manner. (p: *<0.05, **<0.01, ***<0.001, ****<1E-4).

FIG. 39 shows the ASO treatment schemes for astrocytes and shows that ASO D5 modulated DDR2 mRNA in iPSC-derived astrocytes in a dose-dependent manner. (p: *<0.05, **<0.01, ***<0.001, ****<1E-4).

FIG. 40 contains a representative western blot image (top) and a graph quantifying the western blot (bottom) showing the protein levels of a STOM polypeptide following the administration of ASOs targeting STOM in H4 cells. Statistics: two-sided t-tests comparing the beta-actin normalized STOM band intensity between STOM-ASO treated against non-targeting-ASO (NT) treated cells. (p: *<0.05, **<0.01, ***<0.001, ****<1E-4).

FIG. 41 contains a representative western blot image (top) and a graph quantifying the western blot (bottom) showing the protein levels of a KANK2 polypeptide following the administration of ASOs targeting KANK2 in H4 cells. Statistics: two-sided t-tests comparing the beta-actin normalized KANK2 band intensity between KANK2-ASO treated against non-targeting-ASO (NT) treated cells. (p: *<0.05, **<0.01, ***<0.001, ****<1E-4).

### DETAILED DESCRIPTION

This document provides methods and materials for treating PSP. For example, this document provides methods and materials for administering one or more inhibitors of a glial polypeptide to a mammal (e.g., a human) having PSP to treat the mammal. As described herein, glial polypeptides such as DDR2, KANK2, and STOM can be upregulated in PSP. In some cases, one or more inhibitors of a glial polypeptide can be used to reduce or slow the progression of PSP. For example, one or more inhibitors of a glial polypeptide can be administered to a mammal having PSP to reduce or slow the progression of PSP. In some cases, one or more inhibitors of a glial polypeptide can be used to reduce or slow neurodegeneration (e.g., tau-related neurodegeneration) within the brain of a mammal (e.g., a human) having PSP. In some cases, one or more inhibitors of a glial polypeptide can be used to reduce a level of tau polypeptides (e.g., 4-repeat tau polypeptides) within the brain of a mammal (e.g., a human) having PSP.

In some cases, one or more (e.g., one, two, three, four, or more) inhibitors of a glial polypeptide can be used to reduce the severity of one or more symptoms of PSP. For example, one or more inhibitors of a glial polypeptide can be administered to a mammal (e.g., a human) in need thereof (e.g., a human having PSP) to reduce the severity of one or more symptoms of the PSP. Examples of symptoms of PSP include, without limitation, loss of balance (e.g., loss of balance while walking), inability to control eye movements (e.g., inability to look downward), blurring vision, double vision, problems with swallowing, stiffness (e.g., stiffness of the neck), awkward movements, slow or slurred speech, sensitivity to bright light, sleep disturbances, dizziness, and cognitive impairment. In some cases, the methods and materials described herein can be effective to reduce the severity of one or more symptoms of PSP in a mammal having PSP by, for example, 10, 20, 30, 40, 50, 60, 70, 80, 90, 95, or more percent.

In some cases, one or more (e.g., one, two, three, four, or more) inhibitors of a glial polypeptide can be used to reduce or slow the progression of PSP. For example, one or more inhibitors of a glial polypeptide can be administered to a mammal (e.g., a human) in need thereof (e.g., a human having PSP) to reduce or slow the progression of PSP in the mammal. In some cases, the methods and materials described herein can be effective to reduce or slow the progression of PSP in a mammal having PSP by, for example, 10, 20, 30, 40, 50, 60, 70, 80, 90, 95, or more percent. In some cases, the methods and materials described herein can be effective to slow the progression of PSP in a mammal having PSP by, for example, at least 6 months (e.g., about 6 months, about 8 months, about 10 months, about 1 year, about 1.5 years, about 2 years, about 2.5 years, about 3 years, about 4 years, about 5 years, or more).

In some cases, one or more (e.g., one, two, three, four, or more) inhibitors of a glial polypeptide can be used to reduce or eliminate a level of tau polypeptides (e.g., 4-repeat tau polypeptides). For example, one or more inhibitors of a glial polypeptide can be administered to a mammal (e.g., a human) in need thereof (e.g., a human having PSP) to reduce a level of tau polypeptides (e.g., 4-repeat tau polypeptides) within the brain of the mammal. In some cases, the methods and materials described herein can be effective to reduce a level of tau polypeptides (e.g., 4-repeat tau polypeptides) within the brain of a mammal having PSP by, for example, 10, 20, 30, 40, 50, 60, 70, 80, 90, 95, or more percent. For example, one or more inhibitors of a glial polypeptide can be administered to a mammal (e.g., a human) in need thereof (e.g., a human having PSP) to reduce a level of tau polypeptides (e.g., 4-repeat tau polypeptides) within the brain of the mammal. In some cases, the methods and materials described herein can be effective to reduce a level of tau polypeptides (e.g., 4-repeat tau polypeptides) within the brain of a mammal having PSP by, for example, 10, 20, 30, 40, 50, 60, 70, 80, 90, 95, or more percent.

In some cases, one or more (e.g., one, two, three, four, or more) inhibitors of a glial polypeptide can be used to reduce or slow neurodegeration. For example, one or more inhibitors of a glial polypeptide can be administered to a mammal (e.g., a human) in need thereof (e.g., a human having PSP) to reduce or slow neurodegeration in the brain of the mammal. In some cases, the methods and materials described herein can be effective to reduce or slow neurodegeration in the brain of a mammal having PSP by, for example, 10, 20, 30, 40, 50, 60, 70, 80, 90, 95, or more percent.

Any type of mammal having PSP can be treated as described herein. Examples of mammals that can have PSP and can be treated as described herein include, without limitation, humans, non-human primates such as monkeys, dogs, cats, horses, cows, pigs, sheep, rabbits, mice, and rats.

In some cases, the methods described herein can include identifying a mammal (e.g., a human) as having PSP. Any appropriate method can be used to identify a mammal as having PSP. For example, physical examination (e.g., to evaluate particular body movements, eye movement, gait, muscle tone, and posture), cognitive function assessments, imaging techniques (e.g., magnetic resonance imaging (MRI) and positron emission tomography (PET) scanning) to detect shrinkage in specific regions of the brain associated with PSP, and/or laboratory tests (e.g., for total tau and/or phosphorylated tau levels in the CSF and/or plasma) can be used to identify a human or other mammal as having PSP.

A mammal (e.g., a human) having PSP can be administered or instructed to self-administer one or more (e.g., one, two, three, four, or more) inhibitors of a glial polypeptide. An inhibitor of a glial polypeptide can inhibit any appropriate glial polypeptide. Examples of glial polypeptides that can be targeted by an inhibitor of a glial polypeptide to treat PSP as described herein include, without limitation, DDR2 polypeptides, and KANK2 polypeptides, STOM polypeptides.

In some cases, a mammal (e.g., a human) having PSP can be administered or instructed to self-administer one or more (e.g., one, two, three, four, five, or more) inhibitors of a DDR2 polypeptide to treat PSP within the mammal. An inhibitor of a DDR2 polypeptide can be an inhibitor of DDR2 polypeptide activity (e.g., anti-DDR2 antibodies such as neutralizing anti-DDR2 antibodies and small molecules that target a DDR2 polypeptide) or an inhibitor of DDR2 polypeptide expression (e.g., nucleic acid molecules designed to induce RNA interference (RNAi) of DDR2 polypeptide expression such as antisense oligonucleotides (ASOs), siRNA molecules, and shRNA molecules). Examples of inhibitors of a DDR2 polypeptide that can be used to treat PSP as described herein can be as shown in Table 1.

TABLE 1
Inhibitors of DDR2 polypeptides.

| Inhibitor | Catalog No./Brand Name/Sequence |
|---|---|
| Nilotinib | TASIGNA ® |
| Saracatinib | AZD-0530 |
| Erlotinib | Tarceva |
| Dasatinib | SPRYCEL ® |
| Sitravatinib | MGCD516 |
| Regorafenib | STIVARGA ® |
| Imatinib | Gleevec and Glivec |
| Sorafenib | NEXAVAR ® |
| ASO | CAGAACTCAAGATAGGCAGC (SEQ ID NO:1) |
| ASO | GTGAAATTCCTGATGCGGTC (SEQ ID NO:2) |
| ASO | GAAGTAGCACTGTACCTCCT (SEQ ID NO:3) |
| ASO | ACAGAGTCAGGACAAATGGC (SEQ ID NO:4) |
| ASO | CACTCTTGCAGCAGACATTC (SEQ ID NO:5) |
| ASO | AGAATCAAGGACTCTCGGAG (SEQ ID NO:6) |
| ASO | AGGTAAACTGGTGAGGGCAA (SEQ ID NO:7) |
| ASO | AAGGTACTTCATGCCAGAGG (SEQ ID NO:8) |
| ASO | CTGCTCATTCCAAAGTCAGC (SEQ ID NO:9) |
| ASO | GCACTGACATCTAGGGCAAA (SEQ ID NO:10) |
| nucleic acid encoding a shRNA | TATTGTTGAACATGCTAGA (SEQ ID NO:11) |
| nucleic acid encoding a shRNA | TCGTATCTCTTCTCCAGCA (SEQ ID NO:12) |
| nucleic acid encoding a shRNA | TCTTCGTATCTCTTCTCCA (SEQ ID NO:13) |
| nucleic acid encoding a shRNA | AATTGATCTTGTACATGGG (SEQ ID NO:14) |
| nucleic acid encoding a shRNA | TGCTGTAGTGAACTTGCCC (SEQ ID NO:15) |
| nucleic acid encoding a shRNA | TCATGGTAGTGAAATTCCT (SEQ ID NO:16) |
| Antibody | Monoclonal anti-DDR2 antibody, clone 3B11E4 |
| Antibody | Monoclonal anti-DDR2 antibody, clone HL1107 |
| Antibody | Monoclonal anti-DDR2 antibody, clone ARC0958 |
| Antibody | Monoclonal anti-DDR2 antibody, clone 1E9A10 |
| Antibody | Monoclonal anti-DDR2 antibody, clone 2B12.1 |

In some cases when an inhibitor of a DDR2 polypeptide is a nucleic acid molecule designed to induce RNAi of DDR2 polypeptide expression, the nucleic acid molecule can include one or more modified nucleotides. Examples of modified nucleotides that can be present in a nucleic acid molecule designed to induce RNAi of DDR2 polypeptide expression include, without limitation, 2′-O-methoxyethyl(2′MOE) nucleotides, 2′-O-methyl(2′OMe) nucleotides, locked nucleic acids (LNAs), and methylated nucleotides (e.g., 5-methyl cytosines (MeCs)).

In some cases when an inhibitor of a DDR2 polypeptide is a nucleic acid molecule designed to induce RNAi of DDR2 polypeptide expression, nucleotides within the nucleic acid molecule can be linked by a phosphorothioate bond.

Examples of nucleic acid molecules designed to induce RNAi of DDR2 polypeptide expression (e.g., SEQ ID NOs: 1-16) containing one or more modified nucleotides and/or one or more phosphorothioate bonds are shown in Example 3.

Additional inhibitors of a DDR2 polypeptide (e.g., nucleic acid molecules designed to induce RNAi against DDR2 polypeptide expression) can be designed based on any appropriate nucleic acid (e.g., a messenger RNA (mRNA)) encoding a DDR2 polypeptide sequence. Examples of nucleic acids encoding a DDR2 polypeptide sequence include, without limitation, those set forth in National Center for Biotechnology Information (NCBI) accession no. NM 006182.4.

In some cases, an inhibitor of a DDR2 polypeptide can be as described elsewhere (see, e.g., Hebron et al., J. Neuroimmunol., 311:1-9 (2017)).

In some cases, a mammal (e.g., a human) having PSP can be administered or instructed to self-administer one or more (e.g., one, two, three, four, five, or more) inhibitors of a KANK2 polypeptide to treat PSP within the mammal. An inhibitor of a KANK2 polypeptide can be an inhibitor of KANK2 polypeptide activity (e.g., anti-KANK2 antibodies such as neutralizing anti-KANK2 antibodies and small molecules that target a KANK2 polypeptide) or an inhibitor of KANK2 polypeptide expression (e.g., nucleic acid molecules designed to induce RNAi of KANK2 polypeptide expression such as ASOs, siRNA molecules, and shRNA molecules). Examples of inhibitors of a KANK2 polypeptide that can be used to treat PSP as described herein can be as shown in Table 2.

TABLE 2
Inhibitors of KANK2 polypeptides.

| Inhibitor | Catalog No./Sequence |
|---|---|
| ASO | TCTCCAGAGCACCATACTGA (SEQ ID NO:17) |
| ASO | CCTCCGTGTTTGATCCTGAT (SEQ ID NO:18) |
| ASO | CACTTGATGTTCATGCGGGA (SEQ ID NO:19) |
| ASO | GCAAGCCAGTCTACATCAAG (SEQ ID NO:20) |
| ASO | CCCTCAGACACCCACAATTA (SEQ ID NO:21) |
| ASO | CGTACCGTGATCAGAAAGTG (SEQ ID NO:22) |
| ASO | CCCTGTCTCTTGAAGTTTGC (SEQ ID NO:23) |
| ASO | TCTCTGTGCTGTCGTTGTCT (SEQ ID NO:24) |
| ASO | ATCCATCCATCCATCCTGCT (SEQ ID NO:25) |
| ASO | CGTCCTGTCTTGCTTTGTCT (SEQ ID NO:26) |
| Antibody | Polyclonal anti-KANK2 antibody, Invitrogen Cat #PA5-116620 |
| Antibody | Polyclonal anti-KANK2 antibody, Proteintech Cat #21733-1-AP |
| Antibody | Polyclonal anti-KANK2 antibody, Invitrogen Cat # PA5-34539 |
| Antibody | Polyclonal anti-KANK2 antibody, Invitrogen Cat # PA5-100585 |
| Antibody | Polyclonal anti-KANK2 antibody, Invitrogen Cat # PA5-53357 |
| Antibody | Polyclonal anti-KANK2 antibody, Proteintech Cat # 20546-1-AP |
| Antibody | Polyclonal anti-KANK2 antibody, Abcam Cat # ab99351 |
| Antibody | Polyclonal anti-KANK2 antibody, Sigma-Aldrich Cat # HPA015643 |

In some cases when an inhibitor of a KANK2 polypeptide is a nucleic acid molecule designed to induce RNAi of KANK2 polypeptide expression, the nucleic acid molecule can include one or more modified nucleotides. Examples of modified nucleotides that can be present in a nucleic acid molecule designed to induce RNAi of KANK2 polypeptide expression include, without limitation, 2′MOE nucleotides, 2′OMe nucleotides, LNAs, and methylated nucleotides (e.g., MeCs).

In some cases when an inhibitor of a KANK2 polypeptide is a nucleic acid molecule designed to induce RNAi of KANK2 polypeptide expression, nucleotides within the nucleic acid molecule can be linked by a phosphorothioate bond.

Examples of nucleic acid molecules designed to induce RNAi of KANK2 polypeptide expression (e.g., SEQ ID NOs: 17-26) containing one or more modified nucleotides and/or one or more phosphorothioate bonds are shown in Example 3.

Additional inhibitors of a KANK2 polypeptide (e.g., nucleic acid molecules designed to induce RNAi against KANK2 polypeptide expression) can be designed based on any appropriate nucleic acid (e.g., a mRNA) encoding a KANK2 polypeptide sequence.

Examples of nucleic acids encoding a KANK2 polypeptide sequence include, without limitation, those set forth in NCBI accession no. NM_001136191.3.

In some cases, a mammal (e.g., a human) having PSP can be administered or instructed to self-administer one or more (e.g., one, two, three, four, five, or more) inhibitors of a STOM polypeptide to treat PSP within the mammal. An inhibitor of a STOM polypeptide can be an inhibitor of STOM polypeptide activity (e.g., anti-STOM antibodies such as neutralizing anti-STOM antibodies and small molecules that target a STOM polypeptide) or an inhibitor of STOM polypeptide expression (e.g., nucleic acid molecules designed to induce RNAi of STOM polypeptide expression such as ASOs, siRNA molecules, and shRNA molecules). Examples of inhibitors of a STOM polypeptide that can be used to treat PSP as described herein can be as shown in Table 3.

TABLE 3
Inhibitors of STOM polypeptides.

| Inhibitor | Catalog No./Sequence |
|---|---|
| ASO | GGACTACGAATTCACAAGGC (SEQ ID NO:27) |
| ASO | ACATTGTTGGAAAGGGAGGC (SEQ ID NO:28) |
| ASO | AACAAACCAGGTCCTTTGGC (SEQ ID NO:29) |
| ASO | GCAATGGTGGTCAGTGTCTG (SEQ ID NO:30) |
| ASO | GGTTCAGAATGAGTCAGTGG (SEQ ID NO:31) |
| ASO | TCTCTGGAGGTAAGGCACAT (SEQ ID NO:32) |
| ASO | TCTGAAACACGGTCTGGATT (SEQ ID NO:33) |
| ASO | ATGCCAGGTTGCTCAGATTC (SEQ ID NO:34) |
| ASO | AGTTTGACAGTATCTGCCCA (SEQ ID NO:35) |
| ASO | ACACGCTCCACCTTTATTCC (SEQ ID NO:36) |
| Antibody | Polyclonal anti-STOM antibody, Sigma-Aldrich Cat # HPA010961 |
| Antibody | Polyclonal anti-STOM antibody, Proteintech Cat # 12046-1-AP |
| Antibody | Polyclonal anti-STOM antibody, Invitrogen Cat # PA5-110443 |
| Antibody | Polyclonal anti-STOM antibody, Abnova Cat # H00002040-D01P |
| Antibody | Polyclonal anti-STOM antibody, Abnova Cat # H00002040-B01P |

In some cases when an inhibitor of a STOM polypeptide is a nucleic acid molecule designed to induce RNAi of STOM polypeptide expression, the nucleic acid molecule can include one or more modified nucleotides. Examples of modified nucleotides that can be present in a nucleic acid molecule designed to induce RNAi of STOM polypeptide expression include, without limitation, 2′MOE nucleotides, 2′OMe nucleotides, LNAs, and methylated nucleotides (e.g., MeCs).

In some cases when an inhibitor of a STOM polypeptide is a nucleic acid molecule designed to induce RNAi of STOM polypeptide expression, nucleotides within the nucleic acid molecule can be linked by a phosphorothioate bond.

Examples of nucleic acid molecules designed to induce RNAi of STOM polypeptide expression (e.g., SEQ ID NOs: 27-36) containing one or more modified nucleotides and/or one or more phosphorothioate bonds are shown in Example 3.

Additional inhibitors of a STOM polypeptide (e.g., nucleic acid molecules designed to induce RNAi against STOM polypeptide expression) can be designed based on any appropriate nucleic acid (e.g., a mRNA) encoding a STOM polypeptide sequence. Examples of nucleic acids encoding a STOM polypeptide sequence include, without limitation, those set forth in NCBI accession no. NM 004099.6.

In some cases, an inhibitor of a STOM polypeptide can be as described elsewhere (see, e.g., Wang et al., Bioorganic & Medicinal Chemistry Letters 30:127600 (2020)).

When an inhibitor of a glial polypeptide is an inhibitor of glial polypeptide expression (e.g., an inhibitor of DDR2 polypeptide expression, an inhibitor of KANK2 polypeptide expression, and an inhibitor of STOM polypeptide expression), one or more nucleic acid molecules designed to induce RNAi of glial polypeptide expression (e.g., ASOs, siRNA molecules, and shRNA molecules) can be in the form of a vector (e.g., a viral vector or a non-viral vector).

When a vector used to deliver one or more nucleic acid molecules designed to induce RNAi of glial polypeptide expression (e.g., ASOs, siRNA molecules, and shRNA molecules) is a viral vector, any appropriate viral vector can be used. A viral vector can be derived from a positive-strand virus or a negative-strand virus. A viral vector can be derived from a virus with a DNA genome or a RNA genome. In some cases, a viral vector can be a chimeric viral vector. In some cases, a viral vector used as described herein can be a viral vector that can infect dividing cells. In some cases, a viral vector used as described herein can be a viral vector that can infect non-dividing cells. Examples of virus-based vectors that can be used to deliver one or more nucleic acid molecules designed to induce RNAi of glial polypeptide expression (e.g., ASOs, siRNA molecules, and shRNA molecules) to a mammal (e.g., a human) as described herein include, without limitation, virus-based vectors based on adenoviruses, AAVs, Sendai viruses, retroviruses, lentiviruses, or herpes simplex viruses (HSVs).

When a vector used to deliver one or more nucleic acid molecules designed to induce RNAi of glial polypeptide expression (e.g., ASOs, siRNA molecules, and shRNA molecules) is a non-viral vector, any appropriate non-viral vector can be used. In some cases, a non-viral vector can be an expression plasmid (e.g., a cDNA expression vector).

In addition to one or more nucleic acid molecules designed to induce RNAi of glial polypeptide expression (e.g., ASOs, siRNA molecules, and shRNA molecules), a vector (e.g., a viral vector or a non-viral vector) can contain one or more regulatory elements operably linked to the nucleic acid molecules designed to induce RNAi of glial polypeptide expression (e.g., ASOs, siRNA molecules, and shRNA molecules). Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in a vector can involve consideration of several factors, including, without limitation, inducibility, targeting, and the level of expression desired. For example, a promoter can be included in a vector to facilitate transcription of a nucleic acid molecule designed to induce RNAi of glial polypeptide expression (e.g., ASOs, siRNA molecules, and shRNA molecules). A promoter can be a naturally occurring promoter or a recombinant promoter. A promoter can be ubiquitous or inducible (e.g., in the presence of tetracycline) and can affect the expression of a nucleic acid encoding a polypeptide in a general or tissue-specific manner (e.g., GFAP promoters and SYN1 promoters). Examples of promoters that can be used to drive expression of a nucleic acid molecule designed to induce RNAi of glial polypeptide expression (e.g., ASOs, siRNA molecules, and shRNA molecules) in cells include, without limitation, GFAP promoters, SYN1 promoters, MAG promoters, CD68 promoters, and FLT1 promoters. As used herein, “operably linked” refers to positioning of a regulatory element relative to a nucleic acid sequence in such a way as to permit or facilitate expression of the nucleic acid sequence. For example, a vector can contain a promoter and nucleic acid sequence designed to induce RNAi of glial polypeptide expression (e.g., ASOs, siRNA molecules, and shRNA molecules). In this case, the promoter is operably linked to a nucleic acid sequence designed to induce RNAi of glial polypeptide expression (e.g., ASOs, siRNA molecules, and shRNA molecules) such that it drives expression of the nucleic acid sequence to induce RNAi of glial polypeptide expression (e.g., ASOs, siRNA molecules, and shRNA molecules) in cells.

In some cases, one or more (e.g., one, two, three, four, or more) inhibitors of a glial polypeptide can be formulated into a composition (e.g., a pharmaceutically acceptable composition) for administration to a mammal (e.g., a human) having PSP. For example, one or more inhibitors of a glial polypeptide can be formulated together with one or more pharmaceutically acceptable carriers (additives), excipients, and/or diluents. Examples of pharmaceutically acceptable carriers, excipients, and diluents that can be used in a composition described herein include, without limitation, cyclodextrins (e.g., beta-cyclodextrins such as KLEPTOSE®), dimethylsulfoxide (DMSO), sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline such as phosphate buffered saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, when a composition containing one or more (e.g., one, two, three, four, or more) inhibitors of a glial polypeptide is administered to a mammal (e.g., a human) having PSP, the composition can be designed for oral or parenteral (including, without limitation, subcutaneous, intramuscular, intrathecal, intravenous, intradermal, intracerebral, intra cisterna magna, or intraventricular injections) administration to the mammal. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

A composition containing one or more (e.g., one, two, three, four, or more) inhibitors of a glial polypeptide can be administered to a mammal (e.g., a human) having PSP locally or systemically. For example, a composition containing one or more (e.g., one, two, three, four, or more) inhibitors of a glial polypeptide can be administered locally by direct injection (e.g., an intracerebral injection, an intrathecal injection, an intra cisterna magna injection, or an intraventricular injection) to the brain of a mammal (e.g., a human).

In some cases, a composition containing one or more (e.g., one, two, three, four, or more) inhibitors of a glial polypeptide can be in the form of a sterile injectable suspension (e.g., a sterile injectable aqueous or oleaginous suspension). This suspension may be formulated using, for example, suitable dispersing or wetting agents (such as, for example, Tween 80) and suspending agents. The sterile injectable preparation can be a sterile injectable solution or suspension in a non-toxic parenterally-acceptable diluent or solvent, for example, as a solution in 1,3-butanediol. Examples of acceptable vehicles and solvents that can be used include, without limitation, saline, mannitol, water, Ringer's solution, and isotonic sodium chloride solution. In addition, sterile, fixed oils can be used as a solvent or suspending medium. In some cases, a bland fixed oil can be used such as synthetic mono- or di-glycerides.

In some cases, a composition containing one or more (e.g., one, two, three, four, or more) inhibitors of a glial polypeptide can be presented in unit-dose or multi-dose containers, for example, sealed ampules and vials, and may be stored in a freeze dried (lyophilized) condition requiring only the addition of the sterile liquid carrier, for example water for injections, immediately prior to use.

A composition containing one or more (e.g., one, two, three, four, or more) inhibitors of a glial polypeptide can be administered to a mammal (e.g., a human) having PSP in any appropriate amount (e.g., any appropriate dose). An effective amount of a composition containing one or more inhibitors of a glial polypeptide can be any amount that can treat a mammal having PSP as described herein without producing significant toxicity to the mammal. In cases where an inhibitor of a glial polypeptide is an inhibitor of DDR2 polypeptide expression (e.g., nucleic acid molecule designed to induce RNA interference of DDR2 polypeptide expression), an effective amount of the inhibitor of a glial polypeptide can be from about 0.01 milligrams per kilogram body weight (mg/kg) to about 25 mg/kg (e.g., from about 0.01 mg/kg to about 20 mg/kg, from about 0.01 mg/kg to about 15 mg/kg, from about 0.01 mg/kg to about 10 mg/kg, from about 0.01 mg/kg to about 5 mg/kg, from about 0.01 mg/kg to about 2 mg/kg, from about 0.01 mg/kg to about 0.5 mg/kg, from about 0.5 mg/kg to about 25 mg/kg, from about 2 mg/kg to about 25 mg/kg, from about 5 mg/kg to about 25 mg/kg, from about 10 mg/kg to about 25 mg/kg, from about 15 mg/kg to about 25 mg/kg, from about 20 mg/kg to about 25 mg/kg, from about 0.5 mg/kg to about 20 mg/kg, from about 1 mg/kg to about 15 mg/kg, from about 5 mg/kg to about 10 mg/kg, from about 0.5 mg/kg to about 2 mg/kg, from about 1 mg/kg to about 5 mg/kg, from about 2 mg/kg to about 8 mg/kg, from about 5 mg/kg to about 10 mg/kg, from about 8 mg/kg to about 15 mg/kg, or from about 10 mg/kg to about 20 mg/kg). In cases where an inhibitor of a glial polypeptide is an inhibitor of STOM polypeptide expression (e.g., nucleic acid molecule designed to induce RNA interference of STOM polypeptide expression), an effective amount of the inhibitor of a glial polypeptide can be from about 0.01 mg/kg to about 25 mg/kg (e.g., from about 0.01 mg/kg to about 20 mg/kg, from about 0.01 mg/kg to about 15 mg/kg, from about 0.01 mg/kg to about 10 mg/kg, from about 0.01 mg/kg to about 5 mg/kg, from about 0.01 mg/kg to about 2 mg/kg, from about 0.01 mg/kg to about 0.5 mg/kg, from about 0.5 mg/kg to about 25 mg/kg, from about 2 mg/kg to about 25 mg/kg, from about 5 mg/kg to about 25 mg/kg, from about 10 mg/kg to about 25 mg/kg, from about 15 mg/kg to about 25 mg/kg, from about 20 mg/kg to about 25 mg/kg, from about 0.5 mg/kg to about 20 mg/kg, from about 1 mg/kg to about 15 mg/kg, from about 5 mg/kg to about 10 mg/kg, from about 0.5 mg/kg to about 2 mg/kg, from about 1 mg/kg to about 5 mg/kg, from about 2 mg/kg to about 8 mg/kg, from about 5 mg/kg to about 10 mg/kg, from about 8 mg/kg to about 15 mg/kg, or from about 10 mg/kg to about 20 mg/kg). In cases where an inhibitor of a glial polypeptide is an inhibitor of KANK2 polypeptide expression (e.g., nucleic acid molecule designed to induce RNA interference of KANK2 polypeptide expression), an effective amount of the inhibitor of a glial polypeptide can be from about 0.01 mg/kg to about 25 mg/kg (e.g., from about 0.01 mg/kg to about 20 mg/kg, from about 0.01 mg/kg to about 15 mg/kg, from about 0.01 mg/kg to about 10 mg/kg, from about 0.01 mg/kg to about 5 mg/kg, from about 0.01 mg/kg to about 2 mg/kg, from about 0.01 mg/kg to about 0.5 mg/kg, from about 0.5 mg/kg to about 25 mg/kg, from about 2 mg/kg to about 25 mg/kg, from about 5 mg/kg to about 25 mg/kg, from about 10 mg/kg to about 25 mg/kg, from about 15 mg/kg to about 25 mg/kg, from about 20 mg/kg to about 25 mg/kg, from about 0.5 mg/kg to about 20 mg/kg, from about 1 mg/kg to about 15 mg/kg, from about 5 mg/kg to about 10 mg/kg, from about 0.5 mg/kg to about 2 mg/kg, from about 1 mg/kg to about 5 mg/kg, from about 2 mg/kg to about 8 mg/kg, from about 5 mg/kg to about 10 mg/kg, from about 8 mg/kg to about 15 mg/kg, or from about 10 mg/kg to about 20 mg/kg). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the PSP in the mammal being treated may require an increase or decrease in the actual effective amount administered.

A composition containing one or more (e.g., one, two, three, four, or more) inhibitors of a glial polypeptide can be administered to a mammal (e.g., a human) having PSP in any appropriate frequency. The frequency of administration can be any frequency that can treat a mammal having PSP without producing significant toxicity to the mammal. For example, the frequency of administration can be from about twice a day to about once a month, once a day to about once every two weeks, or from about once every other day to about once a week. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more (e.g., one, two, three, four, or more) inhibitors of a glial polypeptide can be administered to a mammal (e.g., a human) having PSP for any appropriate duration. An effective duration for administering or using a composition containing one or more inhibitors of a glial polypeptide can be any duration that can treat a mammal having PSP without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, the methods for treating a mammal (e.g., a human) having PSP as described herein (e.g., by administering one or more inhibitors of a glial polypeptide) can include administering to the mammal one or more (e.g., one, two, three, four, or more) inhibitors of a glial polypeptide as the sole active ingredient to treat the mammal. For example, a composition containing one or more inhibitors of a glial polypeptide can include the one or more inhibitors of a glial polypeptide as the sole active ingredient(s) in the composition that is effective to treat a mammal having PSP.

In some cases, the methods for treating a mammal (e.g., a human) having PSP as described herein (e.g., by administering one or more inhibitors of a glial polypeptide) also can include administering to the mammal one or more (e.g., one, two, three, four, five or more) additional agents used to treat one or more symptoms of PSP to the mammal. For example, a combination therapy used to treat PSP can include administering to the mammal (e.g., a human) one or more inhibitors of a glial polypeptide described herein and one or more (e.g., one, two, three, four, five or more) agents used to treat one or more symptoms of PSP. Examples of agents that can be administered to a mammal to treat one or more symptoms of PSP include, without limitation, carbidopa-levodopa (e.g., RYTARY®, SINEMET®, and DUOPA™), pramipexole (e.g., MIRAPEX® ER), rotigotine (e.g., NEUPRO®), apomorphine (e.g., APOKYN®), selegiline (e.g., ZELAPAR™), rasagiline (e.g., AZILECT®), safinamide (e.g., XADAGO™), entacapone (e.g., COMTAN®), opicapone (e.g., ONGENTYS®), tolcapone (e.g., TASMAR®), benztropine (e.g., COGENTIN®), trihexyphenidyl, amantadine (e.g., GOCOVRI®), istradefylline (e.g., NOURIANZ®), onabotulinumtoxinA (e.g., BOTOX®), eye drops, levodopa (L-Dopa), and any combinations thereof. In cases where one or more inhibitors of a glial polypeptide are used in combination with additional agents used to treat one or more symptoms of PSP, the one or more additional agents can be administered at the same time (e.g., in a single composition containing both one or more inhibitors of a glial polypeptide and the one or more additional agents) or independently. For example, one or more inhibitors of a glial polypeptide described herein can be administered first, and the one or more additional agents administered second, or vice versa.

In some cases, the methods for treating a mammal (e.g., a human) having PSP as described herein (e.g., by administering one or more inhibitors of a glial polypeptide) also can include performing one or more (e.g., one, two, three, four, five or more) therapies used to treat PSP on the mammal. For example, a combination therapy used to treat PSP can include administering to the mammal (e.g., a human) one or more (e.g., one, two, three, four, or more) inhibitors of a glial polypeptide described herein and performing one or more (e.g., one, two, three, four, five or more) additional therapies used to treat PSP on the mammal. Examples of therapies used to treat PSP include, without limitation, physical therapy, occupational therapy, facial exercises, and/or gait and balance training. In cases where one or more inhibitors of a glial polypeptide described herein are used in combination with one or more additional therapies used to treat PSP, the one or more additional therapies can be performed at the same time or independently of the administration of one or more inhibitors of a glial polypeptide described herein. For example, one or more inhibitors of a glial polypeptide described herein can be administered before, during, or after the one or more additional therapies are performed.

In certain instances, a course of treatment and the severity of one or more symptoms related to the condition being treated (e.g., PSP) can be monitored. Any appropriate method can be used to determine whether or not the severity of a symptom is reduced. For example, the severity of a symptom of PSP can be assessed using physical examination (e.g., to evaluate particular body movements, eye movement, gait, muscle tone, and posture), cognitive function assessments, imaging techniques (e.g., magnetic resonance imaging (MRI) and positron emission tomography (PET) scanning) to detect shrinkage in specific regions of the brain associated with PSP, and/or laboratory tests (e.g., for total tau and/or phosphorylated tau levels in the CSF and/or plasma) at different time points.

The invention will be further described in the following examples, which do not limit the scope of the invention described in the claims.

### EXAMPLES

### Example 1: Cross Species Systems Biology Discovers Glial DDR2, STOM, and KANK2 as Therapeutic Targets in Progressive Supranuclear Palsy (PSP)

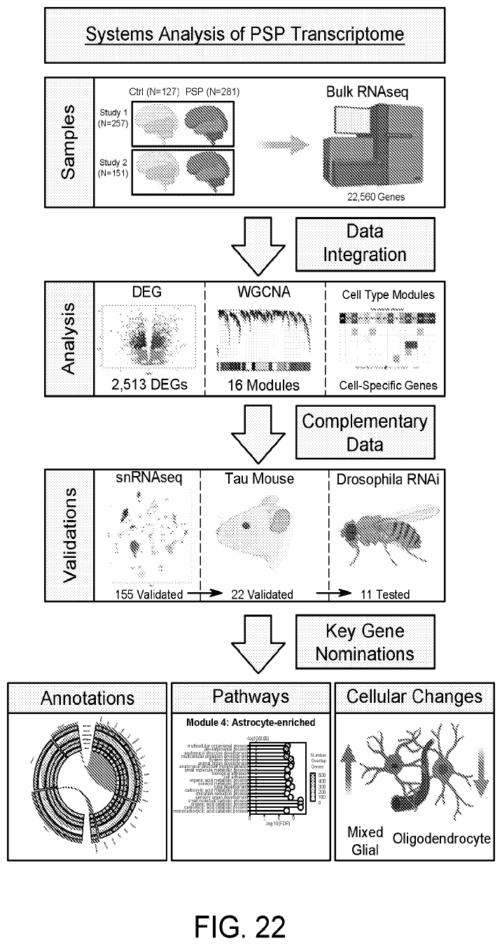

This Example describes the identification of DDR2, STOM, and KANK2 as being overexpressed in subjects with PSP. For example, a systems biology approach was used to integrate multimodal omics and phenotype data across species with experimental validations (FIG. 22).

### Materials and Methods

### Sample Information

RNA was collected from bulk brain tissue of 408 frozen, post-mortem, or temporal cortex (superior temporal gyrus) tissue samples consisting of 127 control and 281 PSP patients from two independent study cohorts (Table 4). All patients received neuropathologic diagnosis of PSP by a single neuropathologist. All PSP samples also underwent neuropathological evaluation for the overall and cell-specific tau lesions (tufted astrocytes (TA), coiled bodies in oligodendrocytes (CB), neurofibrillary tangles (NFT), and tau threads (TauTh)). The semi-quantitative tau pathology scores were transformed and normalized as described elsewhere (see, for example, Allen et al., Acta Neuropathol., 132:197-211 (2016)).

TABLE 4
Demographic information for the bulk RNAseq samples.

| | Variable | | | | | | |
|---|---|---|---|---|---|---|---|
| | Study 1 | | | | | | |
| | Ctrl, | | PSP, | | Overall, | | p- |
| | N = 58 [symbol] | | N = 199 [symbol] | | N = 25 [symbol] | | value² |
| Sex | | | | | | | 0.9 |
| Female | 34 | (59%) | 114 | (57%) | 148 | (58%) | |
| Male | 24 | (41%) | 85 | (43%) | 109 | (42%) | |
| Age | 84 | (79, 92) | 78 | (71, 83) | 79 | (73, 84) | <0.001 |
| RIN | 8.80 | (8.00, 9.20) | 8.10 | (7. [symbol] 0, 8.90) | 8.30 | (7.50, 9.00) | 0.002 |
| Source | | | | | | | <0.001 |
| Banner | 0 | (0%) | 0 | (0%) | 0 | (0%) | |
| Mayo | 15 | (2 [symbol] %) | 199 | (100%) | 214 | ( [symbol] 3%) | |
| UKY | 43 | (74%) | 0 | (0%) | 43 | (17%) | |

| | Variable | | | | | | |
|---|---|---|---|---|---|---|---|
| | Study 2 | | | | | | |
| | Ctrl, | | PSP, | | Overall, | | p- |
| | N = 69 [symbol] | | N = 82 [symbol] | | N = 151 [symbol] | | value² |
| Sex | | | | | | | 0.3 |
| Female | 34 | (49%) | 33 | (40%) | 67 | (44%) | |
| Male | 35 | (51%) | 49 | (60%) | 84 | (5 [symbol] %) | |
| Age | 86 | (78, 89) | 74 | (69, 79) | 78 | (72, 85) | <0.001 |
| RIN | 7.90 | (7.10, 8.50) | 8.40 | (8.10, 8.80) | 8.30 | (7.90, 8.85) | <0.001 |
| Source | | | | | | | <0.001 |
| Banner | 44 | (64%) | 0 | (0%) | 44 | (29%) | |
| Mayo | 25 | (36%) | 82 | (100%) | 107 | (71%) | |
| UKY | 0 | (0%) | 0 | (0%) | 0 | (0%) | |

¹n (%); Median (IQR)
²Pearson's Chi-squared test: Wilcoxon rank sum test: Fisher's exact test [symbol]
indicates data missing or illegible when filed

Controls samples were obtained and were defined as those brain samples having Braak NFT stage of 3.0 or less; CERAD neuritic and cortical plaque densities of 0 (none) or 1 (sparse) and lacking any of the following pathologic diagnosis: AD, Parkinson's disease (PD), DLB, VaD, PSP, motor neuron disease (MND), CBD, Pick's disease (PiD), Huntington's disease (HD), FTLD, hippocampal sclerosis (HipScl) or dementia lacking distinctive histology (DLDH).

Study 1 comprised 199 PSP and 58 control samples, whereas study 2 had 82 PSP and 69 control samples. Within each study cohort, there was well-balanced sex distribution between PSP and controls; however, there were differences for donor age at death, RNA integrity number (RIN) and brain bank source, all of which were adjusted for in the analyses.

For snRNAseq, 18 PSP and 16 control samples that passed quality control (QC) were analyzed. These snRNAseq samples that passed QC had partial overlap with the samples that underwent bulk RNAseq analysis (Table 5).

TABLE 5
Demographic information for the snRNAseq samples.
**Post-QC snRNAseq Sample Demographics**
snRNAseq

| | Control, | | PSP, | | Overall, | | p- |
|---|---|---|---|---|---|---|---|
| | N = 16¹ | | N = 18¹ | | N = 34¹ | | value² |
| Sex | | | | | | | 0.5 |
| Female | 9 | (56%) | 8 | (44%) | 17 | (50%) | |
| Male | 7 | (44%) | 10 | (56%) | 17 | (50%) | |
| Age | 90 | (87, 93) | 86 | (82, 91) | 89 | (83, 92) | 0.10 |
| Bulk RNAseq | | | | | | | <0.001 |
| Overlap | | | | | | | |
| Neither | 9 | (56%) | 2 | (11%) | 11 | (32%) | |
| Study 1 | 0 | (0%) | 16 | (89%) | 16 | (47%) | |
| Study 2 | 7 | (44%) | 0 | (0%) | 7 | (21%) | |

¹n (%); Median (IQR)
²Pearson's Chi-squared test; Wilcoxon rank sum test; Fisher's exact test

### RNAseq of Bulk Human Brain

Raw bulk RNAseq data in study 2 cohort were described elsewhere (see, for example, Wang et al., J. Clin. Invest., 132 (2022)). For study 1, cDNA libraries were prepared using 200 ng of total RNA according to the manufacturer's instructions for the TruSeq RNA Sample Prep Kit v2 (Illumina, San Diego, CA). The concentration and size distribution of the completed libraries were determined using an Agilent Bioanalyzer DNA 1000 chip (Santa Clara, CA) and Qubit fluorometry (Invitrogen, Carlsbad, CA). Libraries were sequenced at six samples per lane, following Illumina's standard protocol using the Illumina cBot and HiSeq 3000/4000 PE Cluster Kit. The flow cells were sequenced as 100×2 paired end reads on an Illumina HiSeq 4000 using the HiSeq 3000/4000 sequencing kit and HCS v3.3.20 collection software. Base-calling was performed using Illumina's RTA version 2.5.2.

snRNAseq of Human Brains

Frozen temporal cortex tissue samples were obtained. Total RNA from ~20 mg collected tissue was isolated to evaluate the quality of tissue. RNA integrity number (RIN) was determined via Agilent 2100 Bioanalyzer using RNA Pico Chip assay, and tissues that have RIN>6.0 were utilized in nuclei isolation and snRNAseq.

For each participant, 100 mg tissue sample was used for nuclei isolation. Samples were homogenized with 25 strokes of loose and tight pestle sequentially using dounce homogenizer in homogenization buffer (0.25 M sucrose, 25 mM KCl, 5 mM MgCl₂, 20 mM tricine-KOH, pH 7.8, 1 mM DTT, 0.15 mM spermine, 0.5 mM spermidine, protease inhibitors, 5 μg/mL actinomycin, 5 U/μL recombinant RNAase inhibitor, and 0.04% BSA). IGEPAL (5%, Sigma) solution was added following stroke with the tight pestle to a final concentration of 0.32%. After 10 additional strokes, the tissue homogenate was filtered using a 30 μm cell strainer. Debris was pelleted by centrifugation at 500 g for 5 minutes and washed with Wash and Storage Buffer (WSB, 1×PBS with 2% BSA and 5 U/μL recombinant RNAase inhibitor). The nuclei-containing supernatant was filtered again with a 30 μm cell strainer, followed by centrifugation at 500 g for 10 minutes. After re-suspending the pellet in 700 μL cold PBS with 5 U/μl RNAse inhibitors, 300 μL debris removal solution (Miltenyi Biotech) was added, and the solution was gently mixed. Another 1 mL WSB was carefully overlaid on top of the nuclei solution. The supernatant was removed after centrifugation at 3000 g for 10 minutes. The nuclei were washed once with WSB and pelleted after centrifugation for 10 minutes at 1000 g.

Isolated nuclei were sorted using fluorescence-activated nuclei sorting (FANS). Human Nuclear Antigen [235-1] (ab191181, Abcam) antibody was applied to the nuclei at 1:50 and incubated for 1 hour on ice. Concurrently, mouse IgG1 and kappa monoclonal isotype were included as controls. Goat anti-mouse 488 secondary antibodies were incubated with the nuclei for 30 minutes on ice. The stained nuclei were reconstituted in WSB and sorted using BD FACSAria II sorter.

After quantifying the sorted nuclei using a hemocytometer in 0.04% trypan blue, a total of 1000 estimated nuclei at 700 nuclei/μL were loaded on the 10× Chromium microchip. Single cell gel beads-in-emulsion (GEMs) were generated by running Single Cell Instrument (10× Genomics) on the sorted nuclei. Chromium Single Cell 3′ Gel Bead and Library Kit v3.1 (10× Genomics, No. 120237) and the Single Index Kit T Set A (10× Genomics, No. 1000213) were used to prepare the single nucleus RNAseq libraries according to the manufacturer's instructions. Qualities of libraries were checked using Agilent High Sensitivity DNA Kit via Agilent 2100 Bioanalyzer.

### Bulk Human Brain RNAseq Analyses and Differential Gene Expression

The bulk RNAseq data in study 2 was aligned and processed as described elsewhere (see, for example, Allen et al., Sci. Data, 3 (2016)). For study 1, raw paired-end reads were processed through MAP-RSeq pipeline v2.0. MAP-RSeq removes reads of low base-calling Phred scores, aligns remaining reads to reference genome hg19 using TopHat aligner v2.0, and counts reads in genes and exons using subread. It obtains QC measures from both pre- and post-alignment reads using RSeQC toolkit and fastQC. Subsequently, samples were identified for exclusion, and were defined as those samples with high RNA degradation, low mappability, discordance between recorded sex and estimated sex, or based on principal component analysis (PCA) such that samples whose PC1 or PC2 are outside mean±/−3×SD.

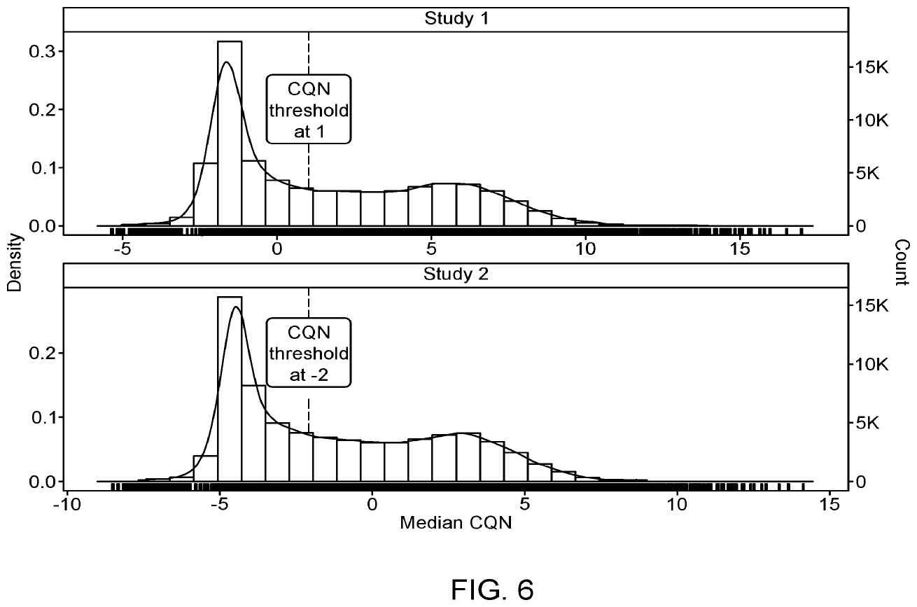

Following exclusion of samples that failed QC, raw RNA read counts from remaining samples were normalized using R package ‘CQN’, which gives library size, gene length, and GC content adjusted expression values in log 2 scale. Based on the bimodal expression distribution, genes with low CQN values (CQN<2 for study 1, CQN<−1 for study 2) were filtered out (FIG. 6). Genes that were expressed in both cohorts were used in the following analysis. Batch effect from the source of the samples was corrected using the combat function from R package ‘sva’. The associations between the batch-corrected, normalized, bulk gene expression, and different clinicopathological traits (PSP diagnosis, TA, CB, NFT, TauTh, and overall pathology) were assessed for study 1 and study 2 separately using a multiple linear model adjusting for technical and biological covariates including sex, age at death, RIN, and sequencing flowcell. Gene expressions were treated as a continuous dependent variable, while the traits of interests were treated as independent variables, coded as binary (PSP=1, control=0) or continuous (quantitative pathology scores) variables. The association effect sizes (regression beta coefficients) in study 1 and study 2 were combined using an inverse-variance weighting meta-analysis, implemented in R package ‘meta’. A fixed-effect model was used for gene-trait associations when Higgin's and Thompson's I² heterogeneity value was less than 0.3, while a random-effect model was used otherwise. Lastly, the Benjamini-Hochberg false discovery rate (FDR) was used to adjust for multiple testing.

### Bulk Human Brain RNAseq Co-Expression Network Analyses

Gene expression networks were constructed using R package ‘WGCNA’. Prior to the network analysis, residuals from the batch-corrected, normalized gene expression data after adjusting for technical and biological covariates (sex, age, RIN, and flowcell number) were generated for study 1 and study 2 separately. The adjacency matrices Aᵢⱼ were separately calculated for study 1 and study 2 using the pairwise biweighted midcorrelation (bicor), whose element aᵢⱼ=bicor(gᵢ,gⱼ). Topological overlap matrices (TOMs) were separately calculated for study 1 and study 2 based on their adjacency matrices as a signed network with a soft power threshold of 12. Subsequently, after applying quantile scaling, the consensus TOM was calculated as the component-wise minimum of the TOMs from study 1 and study 2. The modules were identified using hierarchal clustering and dynamic treecut algorithm provided in the WGCNA package from consensus TOM.

From these initial modules, the final modules were identified as follows. The ME were calculated as the signed first principal component using gene expression of initial modules. The module memberships (MM) were calculated as the biweighted midcorrelation of the ME and gene expression. Modules that are closely related, defined as biweighted midcorrelation of ME higher than 0.8, were merged. Genes were further examined and re-assigned in each module based on their MM. Specifically, genes with negative MM were moved to the background module (module 0), while genes in the initial background modules were reassigned to the module with maximum MM if the maximum MM was greater than 0.5. The ME and MM values were recalculated using the final module definitions.

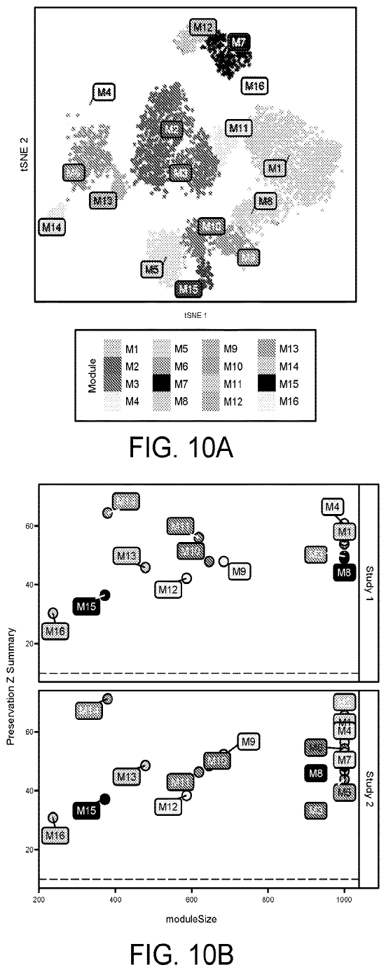

tSNE plots (FIGS. 10A-10B) were made for final modules using gene expression of the combined cohort of study 1 and 2 through R package ‘Rtsne’. In addition, statistics were calculated separately regarding preservedness of final modules in studies 1 and 2 (FIGS. 10A-10B). The module preservations were assessed using the “WGCNA::modulePreservation( )” function, and calculated preservation Z statistics with 100 permutations.

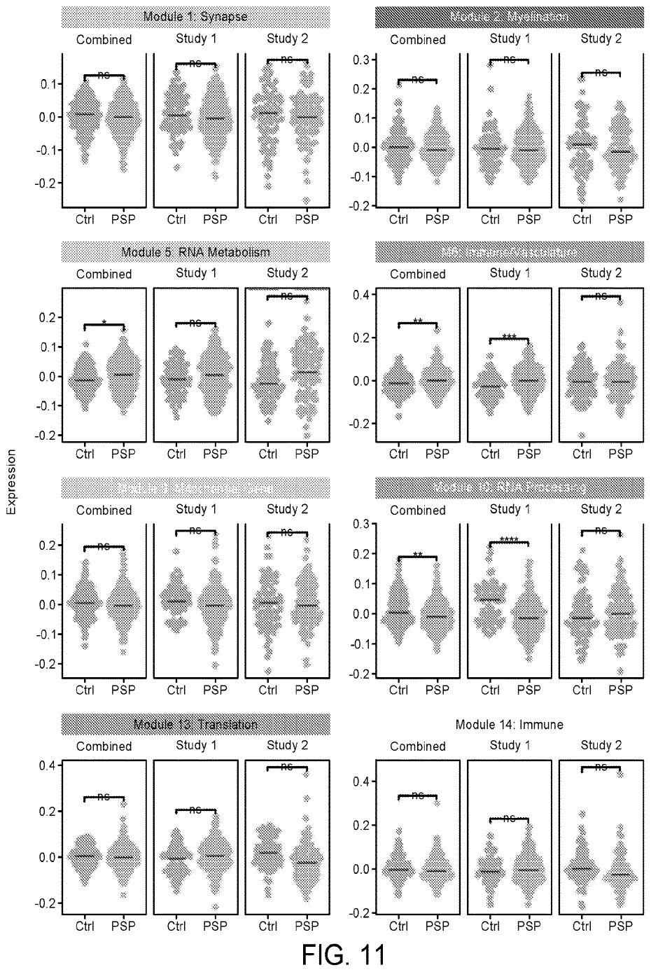

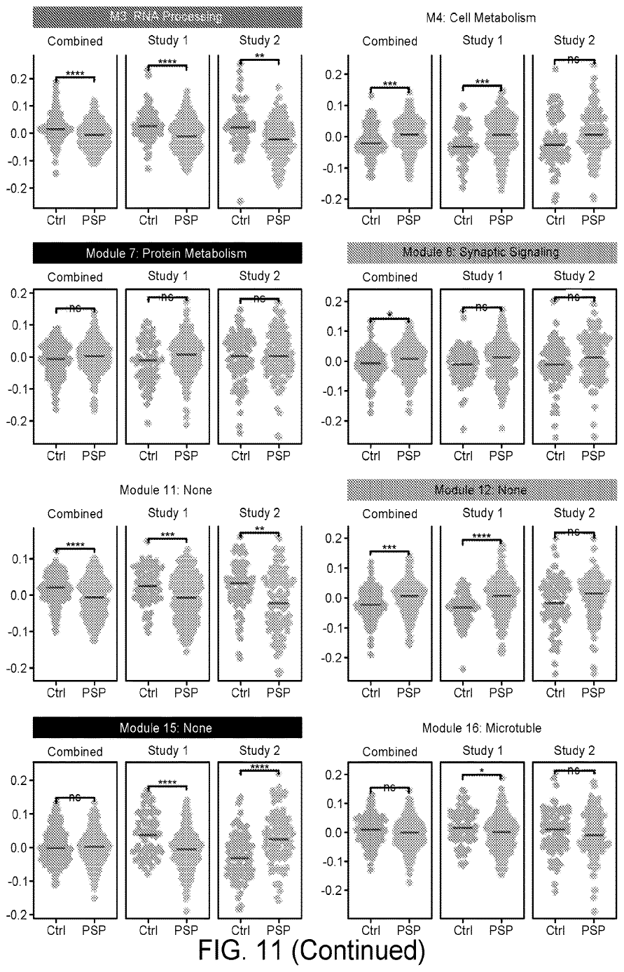

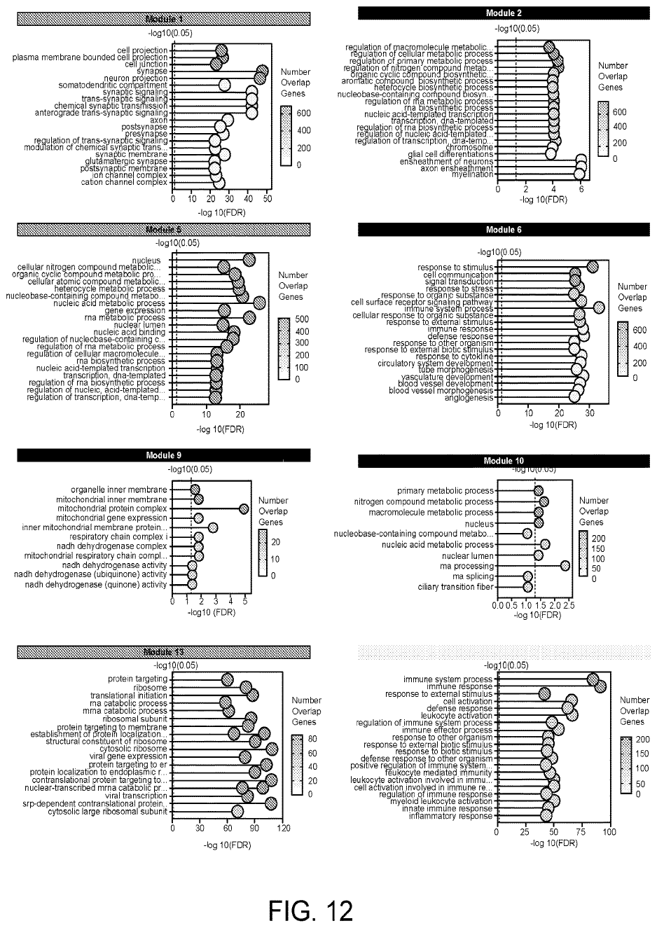

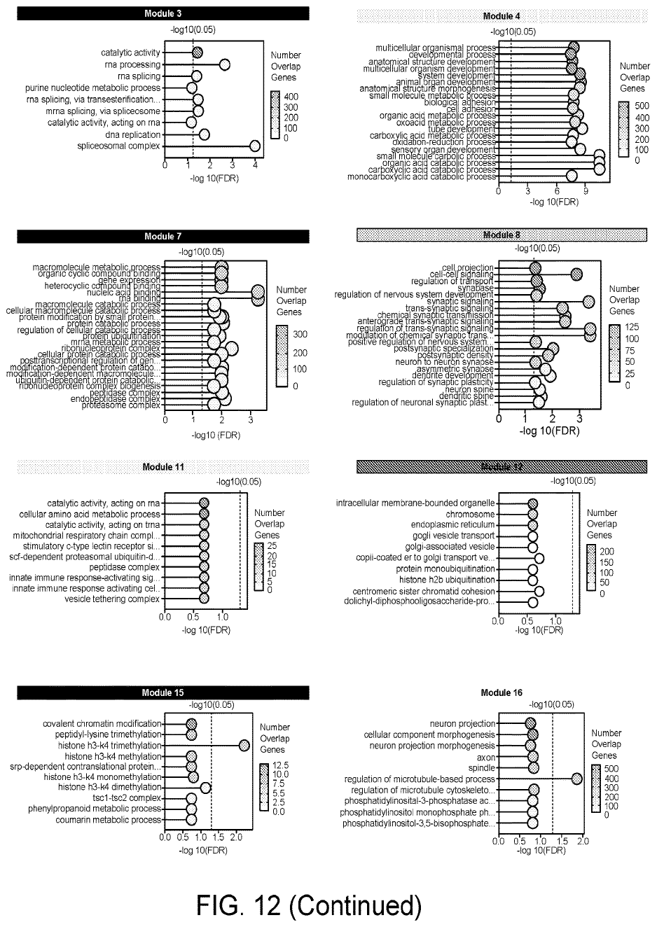

Biweighted midcorrelations between the ME and traits (PSP diagnosis, TA, CB, NFT, TauTh, and overall pathology) were calculated for each module. The association significance p values were adjusted to the number of modules using Bonferroni correction. Based on the module assignment, significant enrichment of the top 1000 cell type marker genes identified using the R package ‘BRETIGEA’ in any of the modules was tested using a one-sided version of Fisher's exact test and a Bonferroni adjusted p-value cut off of 0.05. Additionally, the gene ontology (GO) terms enriched in the modules were calculated using R package ‘anRichment’, which calculates the statistical significance of the overrepresentation of GO terms based on hypergeometric distribution with an FDR-adjusted p-value of 0.05. Modules were manually annotated with the top GO terms from the list (FIGS. 11-12).

Human Brain snRNAseq Validations

Raw snRNAseq data were processed and aligned using Cell Range version 4.0 (10× Genomics). Raw reads were mapped to human reference genome hg38 using STAR aligner. An average of 924 nuclei (standard deviation: 469, N=36) were obtained per sample and quality control was performed for each individual nucleus. Nuclei with more than 10% mitochondrial genes were excluded. Nuclei with an extreme number of detected genes (lower bound 500, upper bound 98^{th} percentile>=9,648.8) or mapped UMIs (lower bound 1,000, upper bound 98^{th} percentile, or >85,906.5) were also removed. Two PSP samples were removed because of low nuclei count. 18 PSP and 16 control samples remained for analyses.

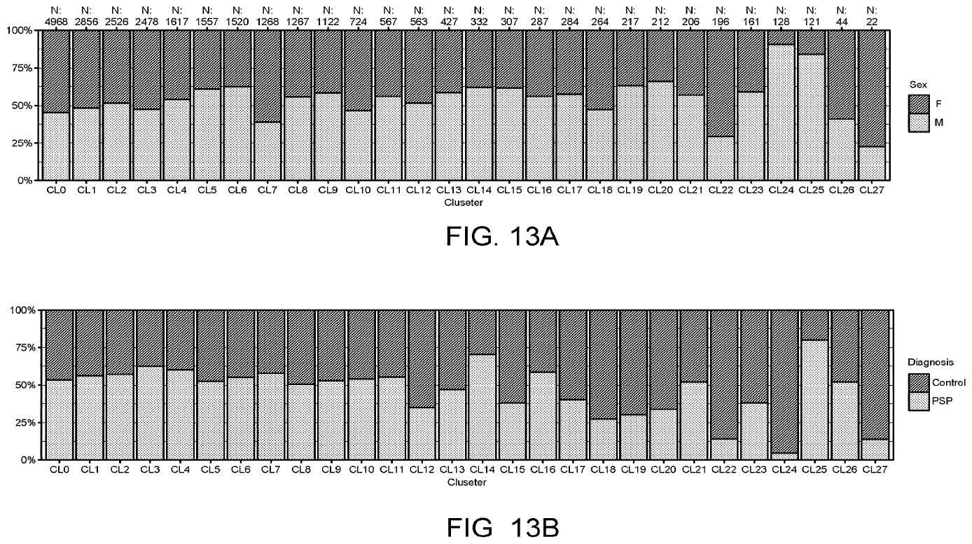

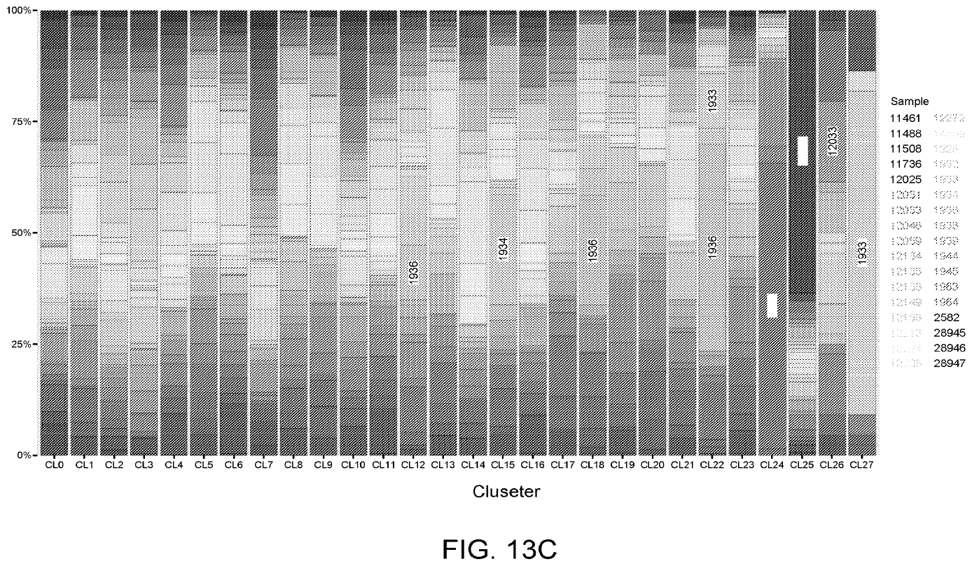

Using the ‘Seurat’ R package, UMI counts from each sample were merged, and library size normalization and log transformation were performed. The dataset was integrated using the Harmony package, and each sample was treated as its own batch. Nuclei were clustered using a Shared Nearest Neighbor (SNN) Graph implemented in Seurat at a resolution of 0.4 with default parameters. Significant enrichment (fold-enriched>5, unadjusted p-value<0.05) of nuclei from any diagnosis, sex, or sample in each cluster was determined (FIGS. 13A-13C).

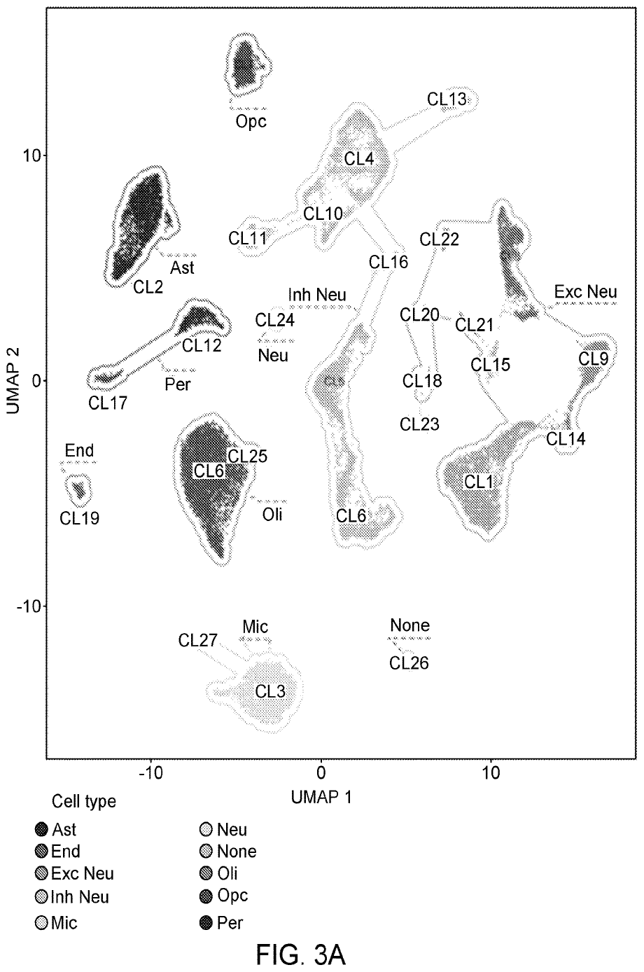

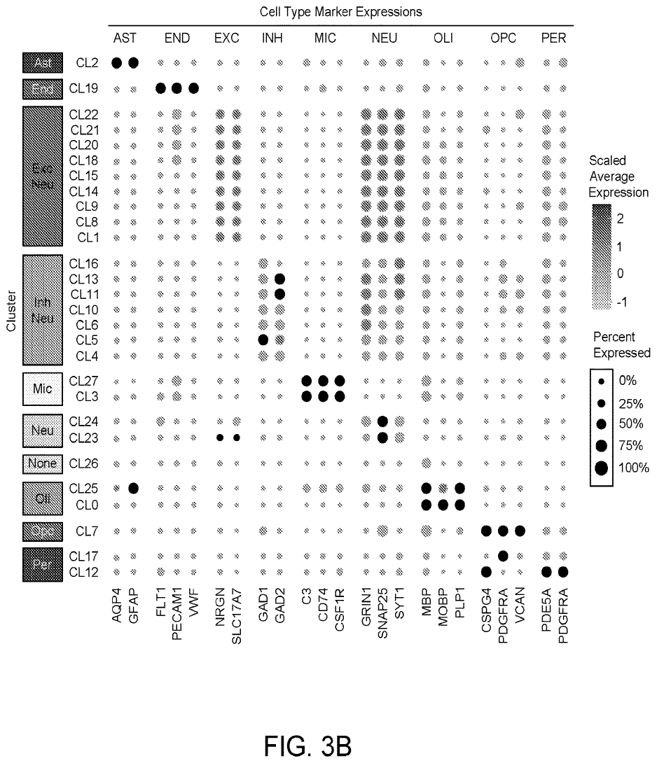

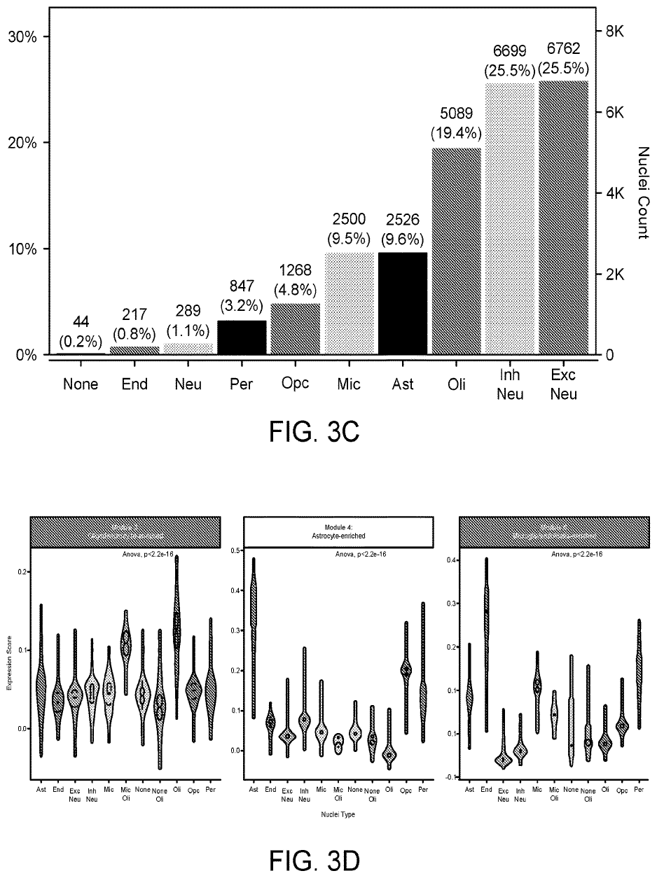

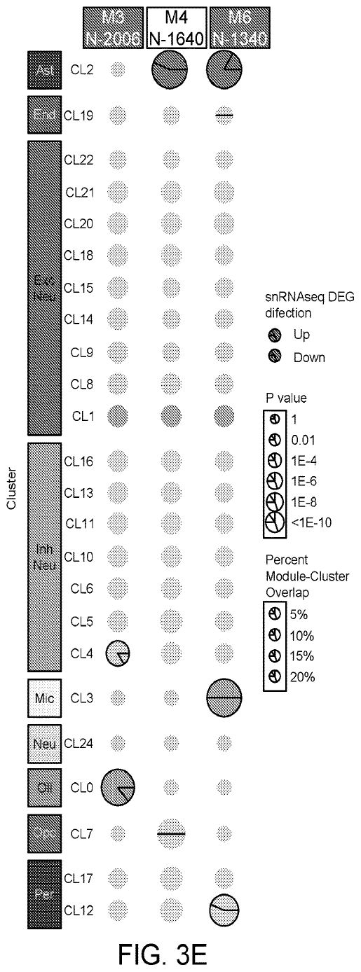

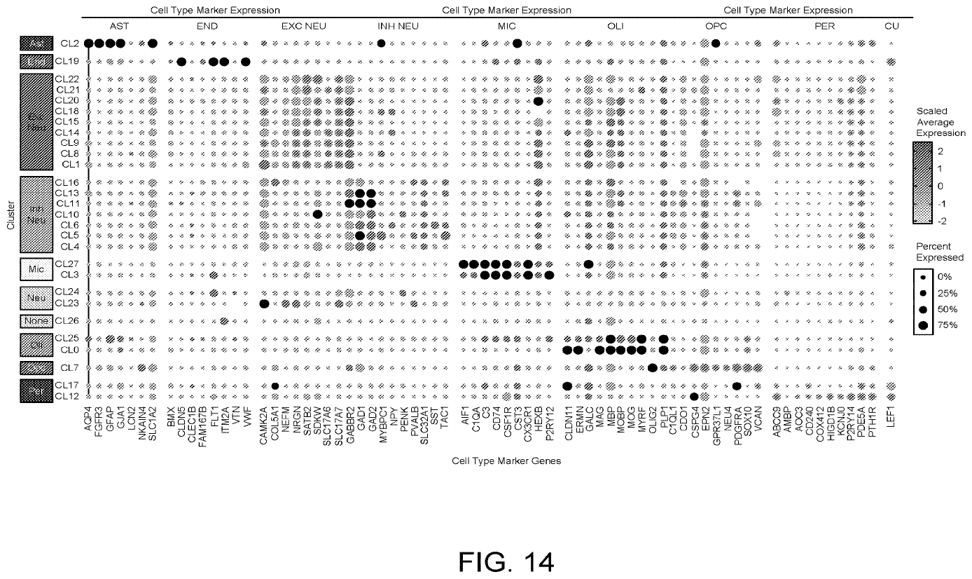

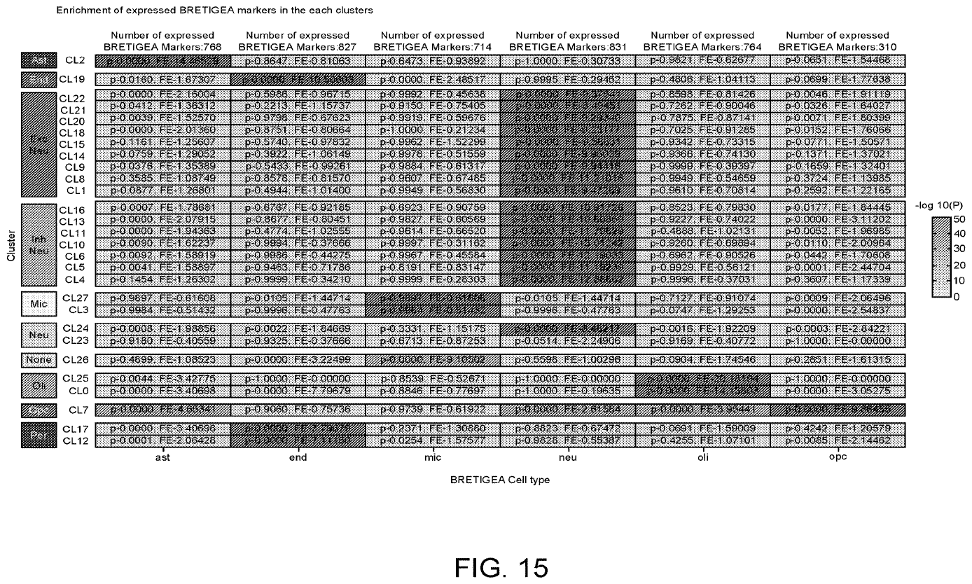

To elucidate cluster cell type, cluster marker genes that were significantly (FDR<0.05) over-expressed (log FC>0.5) and universally expressed (percent nuclei>70%) in each cluster were identified and compared to those of other clusters. Each cluster was annotated based on the overlap of their highly overexpressed cluster-marker genes and known cell-type marker genes curated manually (FIG. 3B; GFAP and AQP4 for astrocyte, VWF, PECAM1, FLT1 for endothelia, NRGN and SLC17A7 for excitatory neurons, GAD1 and GAD2 for inhibitory neuron, C3, CD74, and CSF1R for microglia, GRIN1, SNAP25, and SYT1 for neurons, MBP, MOBP, and PLP1 for oligodendrocyte, CSPG4, PDGFRA, and VCAN for oligodendrocyte progenitor cells (opc), and PDE5A and PDGFRB for pericyte). Additional cluster cell type assignment was performed with published databases (FIGS. 14-15).

Genes that were differentially expressed between PSP and control nuclei (differentially expressed genes (DEGs)) were identified for each cluster through a hurdle model implemented in ‘MAST’ R package, with adjustment of sex and age. DEGs were detected (UMI>0) in at least 20% of the nuclei in a cluster. Multiple testing was adjusted for using FDR<0.05. Pathway enrichment analysis used the FUMA GWAS web service (v1.4.0) using all 22,431 expressed snRNAseq genes as background. Multiple testing was adjusted for using Benjamini-Hochberg method. MigSigDB v7.0 was used. A minimal overlap of 2 genes was required.

For each nucleus in the snRNAseq analysis, its expression scores, which reflect the expression levels of a selected set of genes, were calculated as the average expression levels of the module genes based on the module definition from the 16 WGCNA modules using the “AddModuleScore( ) function implemented in Seurat.

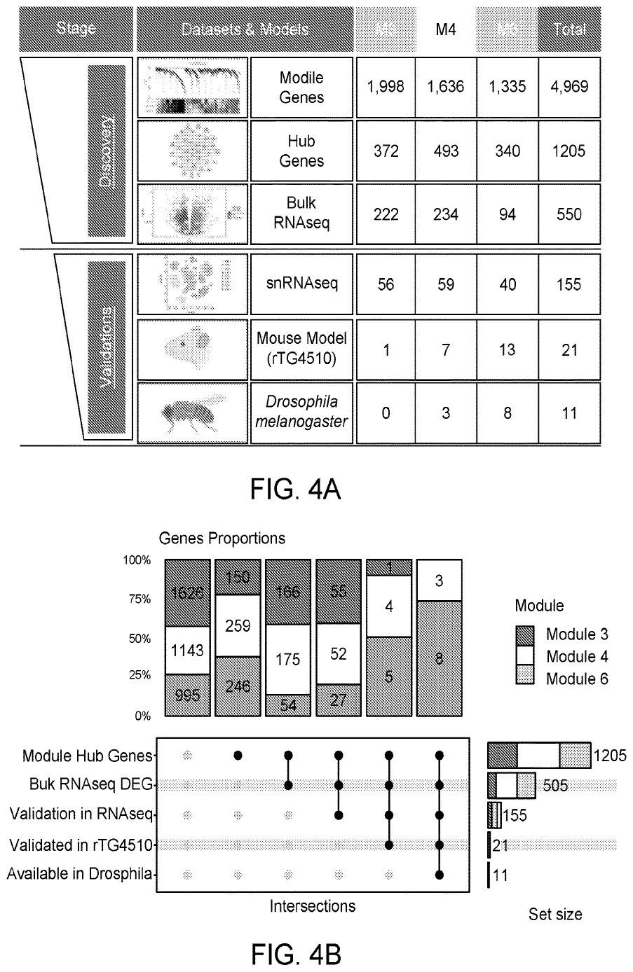

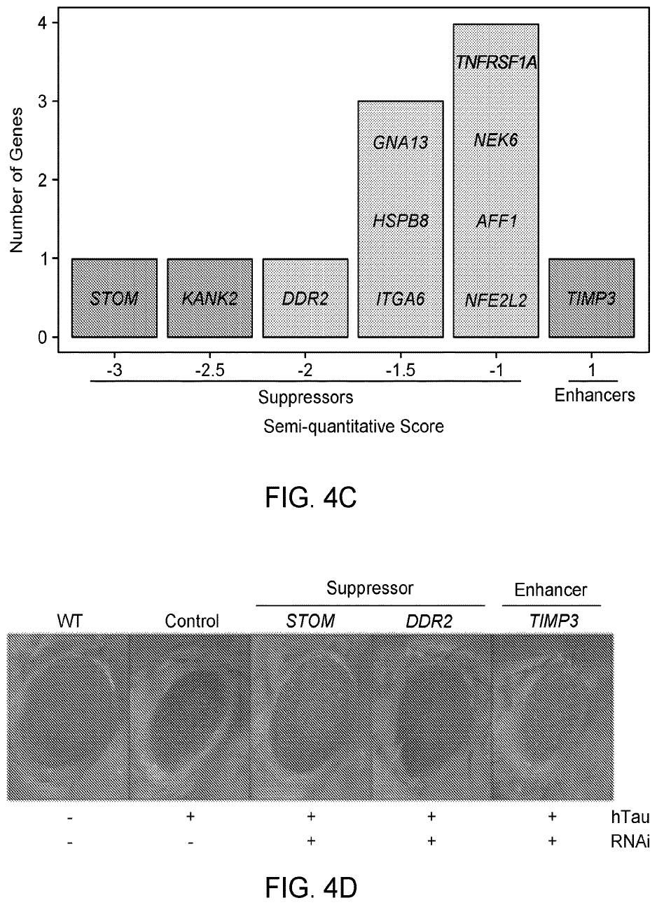

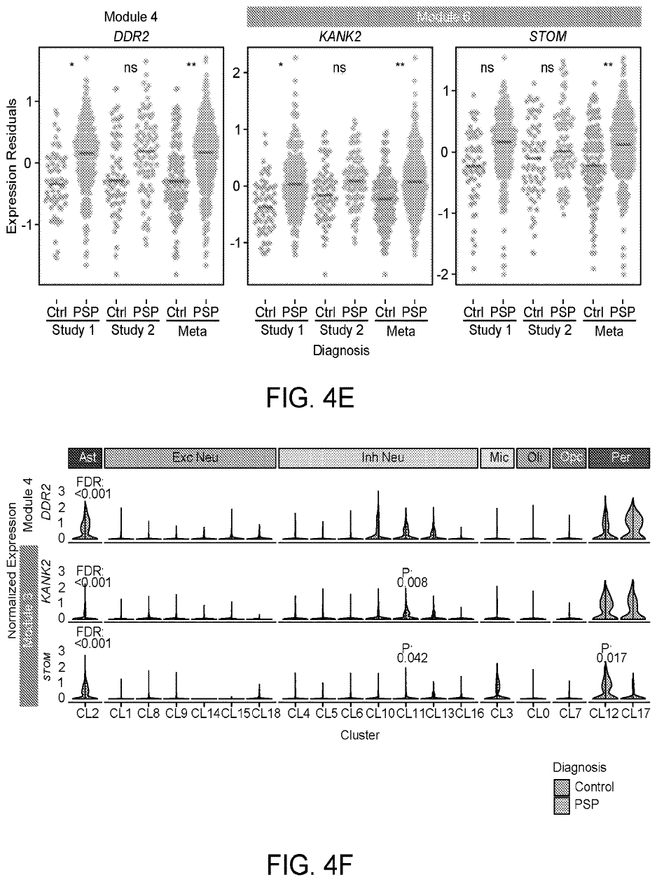

The snRNAseq data was used to validate and filter the bulk RNASeq DEGs based on the prioritization approach (FIG. 4A). The bulk DEGs (FDR<0.05 in the meta-analysis of study 1+2) from the PSP-associated co-expression modules that were also enriched in brain cell types (modules 3, 4 and 6) were focused on. There were 4,969 such DEGs, 550 of which were also highly-connected network hub genes with module membership (MM)>0.7. These 550 significant PSP DEG hub genes were filtered by selecting those that were also significant DEGs in the snRNAseq data cluster corresponding to the bulk module cell-type and that had concordant direction of change between bulk and snRNAseq. Specifically, for M3 candidate genes, genes that were significantly downregulated in any of the oligodendrocyte clusters (CLO or CL26) were selected. For the M4 candidate genes, genes that were upregulated in the astrocyte cluster (CL2) were selected. Lastly, for the M6 candidate genes, genes that were upregulated in any of the microglia (CL3, CL27), endothelia (CL19), pericytes (CL12, CL17), or astrocytes (CL2) clusters were selected.

Validation with rTg4510 Tauopathy Mouse Model Brain RNAseq

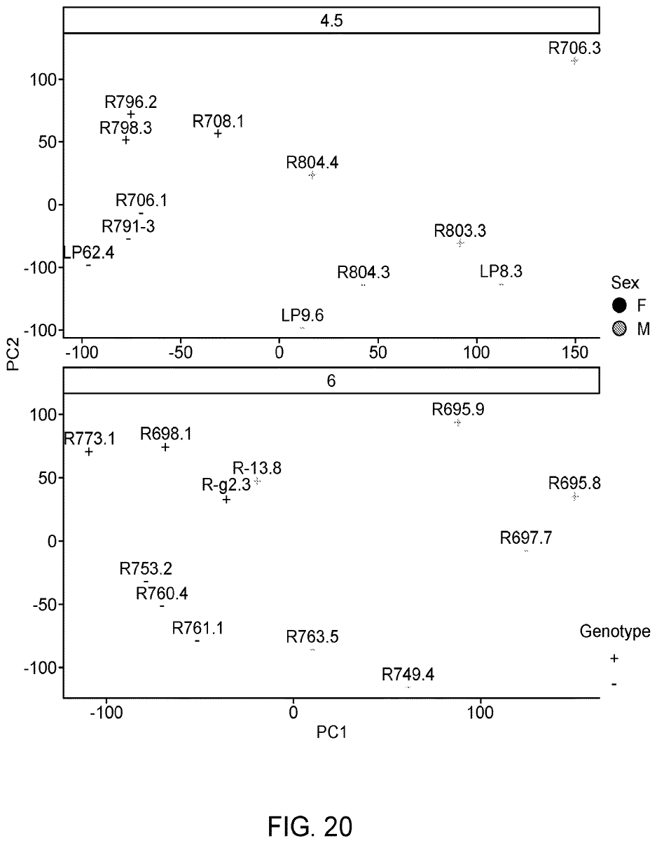

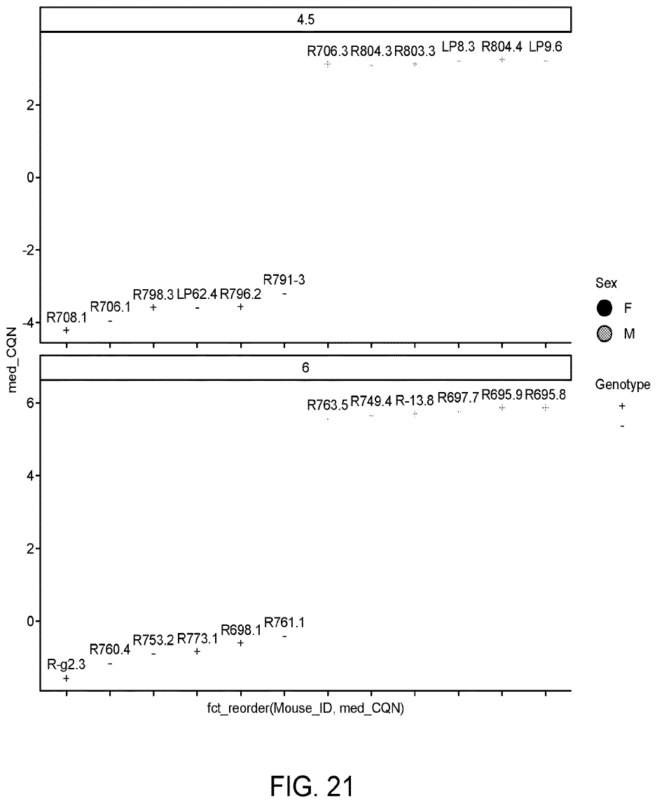

Bulk brain RNAseq data of the tauopathy mouse model rTg4510 was retrieved from the AD Knowledge Portal (syn3157182). Briefly, the dataset consists of bulk RNAseq from forebrain samples of rTg4510 mice overexpressing human P301L tau (4R0N) and wild type non-transgenic (nonTg) littermate control mice. Using the RNAseq data from the 4.5- and 6-month-old (Table 6) mice, the gene expression values were normalized using CQN, followed by QC to check for outliers or mismatched sex (FIGS. 20-21). Read counts between rTg4510 and nonTg mice were compared in each dataset within each age group using a negative binomial generalized linear model implemented in R package ‘edgeR’, with adjustment of sex and RIN. DEGs were identified based on an FDR-adjusted p-value of 0.05. Mouse genes were mapped to their human orthologs using R package ‘biomaRt’ and ensemble version 105.

TABLE 6
Mouse demographic information.

| | rTG4510 Mouse Model | | | |
|---|---|---|---|---|
| | Control, | Transgenic, | Overall, | |
| | N = 12¹ | N = 12¹ | N = 24¹ | p-value² |
| RIN | 7.20 (6.90, | 7.20 (7.07, | 7.20 (7.07, | 0.7 |
| | 7.50) | 7.62) | 7.53) | |
| Sex | | | | >0.9 |
| Female | 6 (50%) | 6 (50%) | 12 (50%) | |
| Male | 6 (50%) | 6 (50%) | 12 (50%) | |
| Age (months) | | | | >0.9 |
| 4.5 | 6 (50%) | 6 (50%) | 12 (50%) | |
| 6 | 6 (50%) | 6 (50%) | 12 (50%) | |

¹Median (IQR); n (%)
²Wilcoxon rank sum test; Pearson's Chi-squared test

Using the mouse DEG information, the 155 human DEGs (FIG. 4A) that were significant, had congruent changes in both bulk and snRNAseq, and were hubs in the prioritized modules M3, M4, and M6 were validated. These human DEGs were considered validated in the mouse model if significant at FDR-adjusted level of 0.05 in either 4.5- or 6-month rTg4510 mouse brains and had the same direction of change as in humans.

Validation with Drosophila Tau Model Experiments

High confidence PSP glial DEGs with significant and congruent changes in human bulk and snRNAseq and rTG4510 mouse model brains were experimentally validated in a Drosophila melanogaster model expressing human wild-type Tau protein. GMR-GAL4/CyO; UAS-hTau/TM3 Drosophila were crossed to RNAi lines and progeny that co-expressed both human Tau and RNAi (GMR-GAL4/+; UAS-hTau/UAS-RNAi, where the UAS-RNAi can be on any chromosome) were selected. The morphology of the Drosophila eyes was compared with the morphology of the control Drosophila expressing the tau protein using a −4 to 4 semi-quantitative scale where 0 indicated no change compared to the tau-expressing control. A positive score indicated enhancement/exacerbation of the well-described eye neurodegeneration pathology, whereas a negative score indicated suppression/rescue of this pathology. A score of 4 indicated that the Drosophila had no eyes, whereas a score of −4 indicated that the eyes were indistinguishable from that of the wild-type control. If the Drosophila fail to enclose (emerge as an adult), the phenotype was recorded as being ‘lethal’.

### Results

PSP Brains have Vast and Replicable Transcriptome Perturbations in Glia-Enriched Genes

To identify perturbed genes in PSP, the bulk gene expression profile from the superior temporal gyrus of temporal cortex (TCX) tissue for 281 neuropathologically confirmed PSP cases and from 127 controls that lack significant pathology were collected and analyzed. Expression data was collected as part of two independent studies (Table 4). All PSP cases have detailed measures of cell-specific tau (CB, NFT, TA, and TauTh) and overall degree of neuropathology quantified from multiple brain.

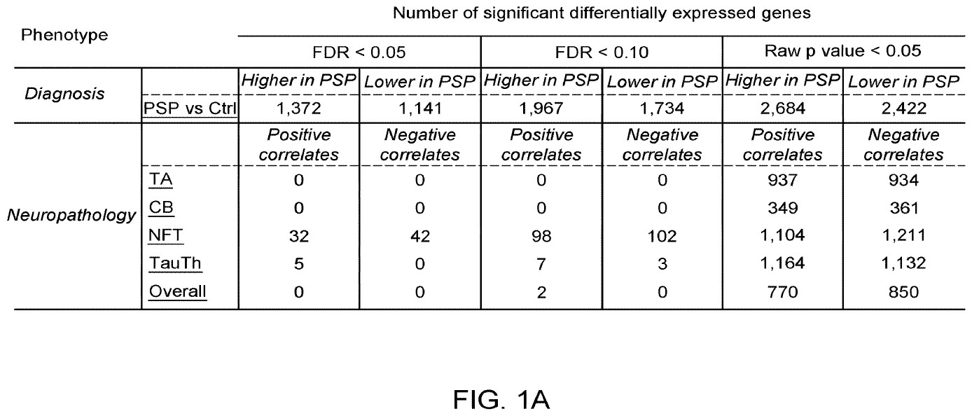

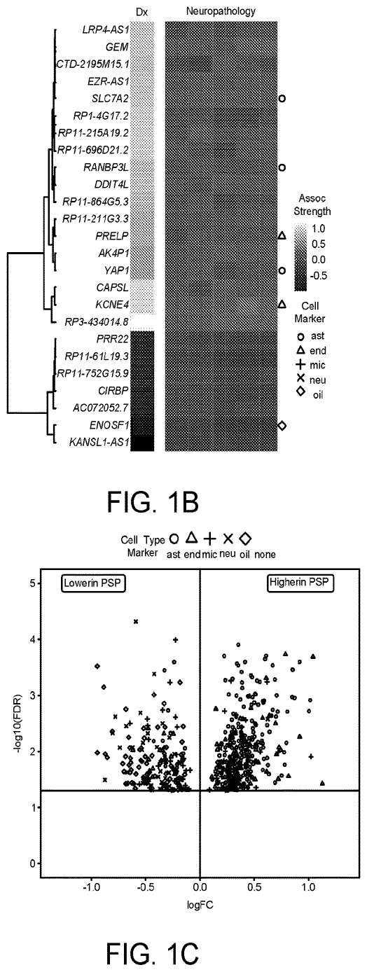

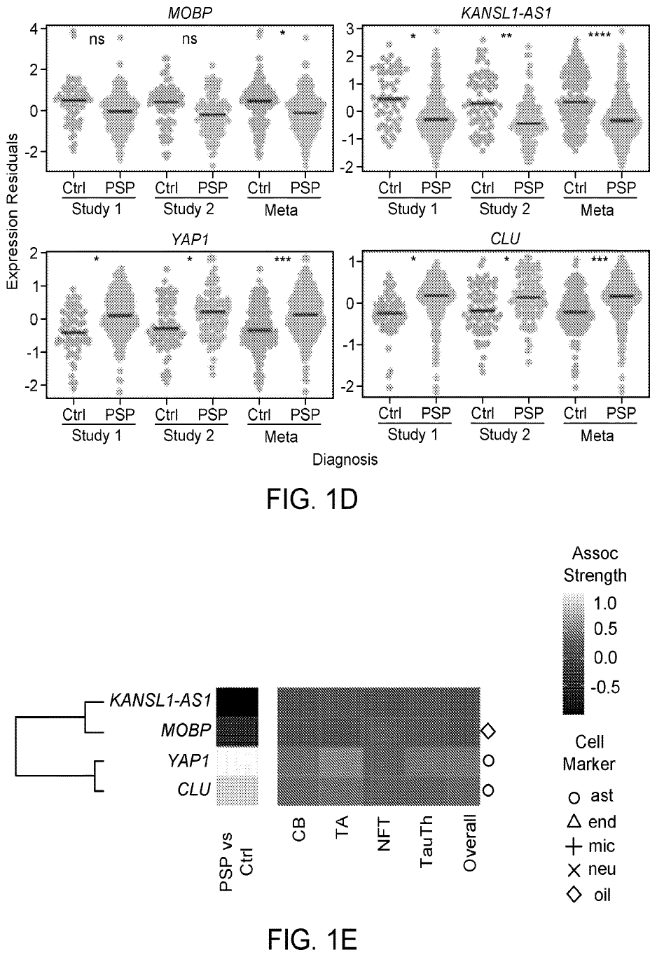

After quality control (QC), a total of 22,560 unique genes were detected in both studies, the majority (67%) of which are protein coding (Table 7, FIG. 6). To identify genes associated with PSP diagnosis, the brain gene expression levels between PSP cases and controls were compared in each dataset separately using multiple linear regression adjusting for relevant covariates, and the results were combined using an inverse-variance meta-analysis model. Using a similar approach, the association of brain gene expression levels with the severity of tau neuropathologies measured within the 281 PSP cases was also analyzed. Compared to controls, 2,513 genes were differentially expressed in PSP brains at an FDR-adjusted p-value of 0.05 (FIG. 1A), suggesting extensive transcriptional dysregulation in PSP brains at bulk tissue level, even in a brain region relatively spared from gross tau pathology. In terms of neuropathology, the greatest number of associations were detected with NFT (74 genes), while 6 gene levels were associated with TauTh (FIG. 1B). Using less stringent significance thresholds (unadjusted p value<0.05), associations were also observed between gene expression levels with other tau neuropathologic lesions in PSP (FIG. 1A).

TABLE 7
Type of detected genes

| Characteristic | | |
|---|---|---|
| Gene Type | N = 22,560¹ | |
| protein_coding | 15,151 | (67%) |
| antisense | 2,717 | (12%) |
| pseudogene | 1,889 | (8.4%) |
| lincANA | 1,619 | (7.2%) |
| processed_transcript | 327 | (1.4%) |
| less than 1% | 857 | (3.8%) |

¹n (%)

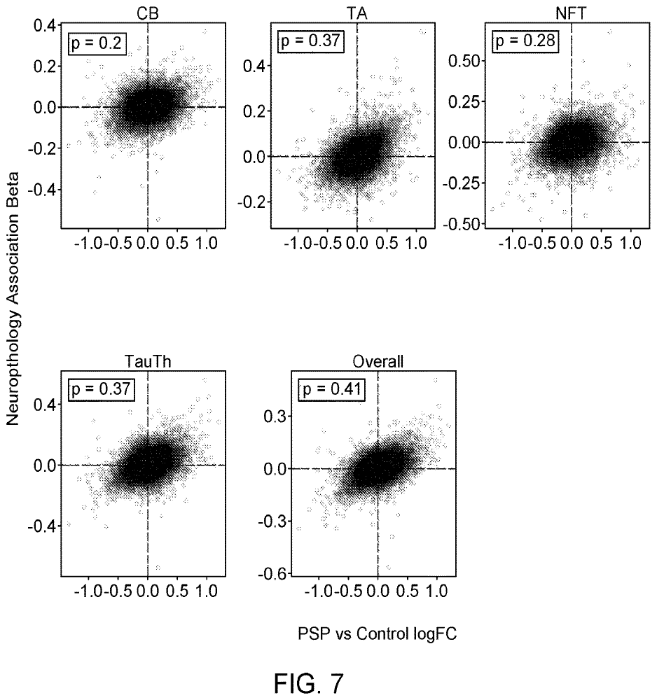

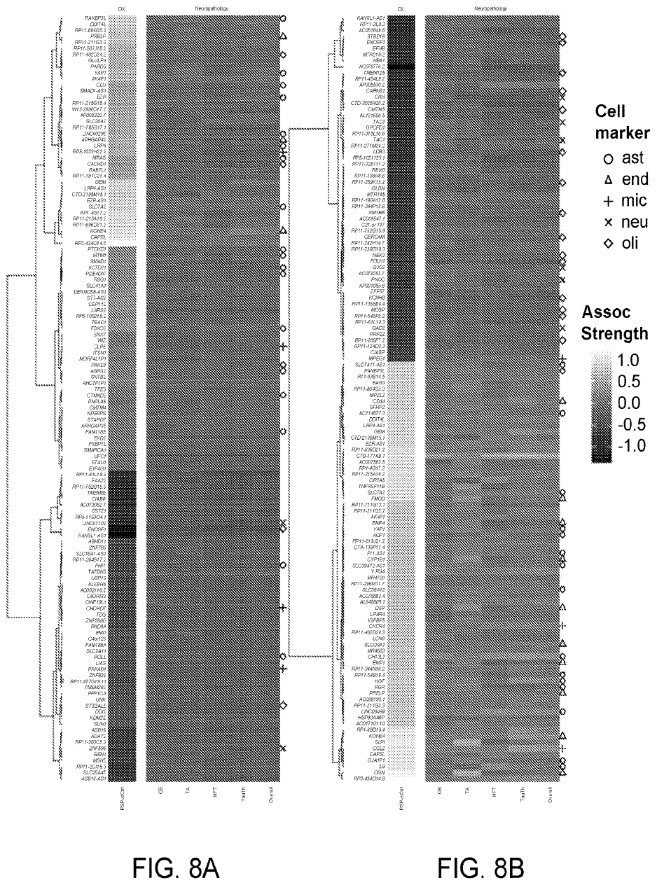

The genes that had the top PSP vs. control brain expression changes (defined as lowest 5% FDR and top 5% of the absolute value of the log FC) were reviewed (FIG. 1B). Biologically congruent gene expression associations with both diagnosis and neuropathology were observed. Specifically, genes that had higher expression in PSP than in controls were also positively correlated with tau neuropathologies, and genes that had down-regulated expression in PSP had negative neuropathology correlations (FIGS. 1B and 7). Among all DEGs (FDR<0.05), there was cell-type specificity as many of the PSP up-regulated genes are also marker genes for astrocytes (p<0.001, 229 genes) or endothelia (p=0.013, 63 genes), whereas many of the PSP down-regulated genes are oligodendrocyte-specific (p<0.001, 77 genes) (FIGS. 1B-1C and 8).

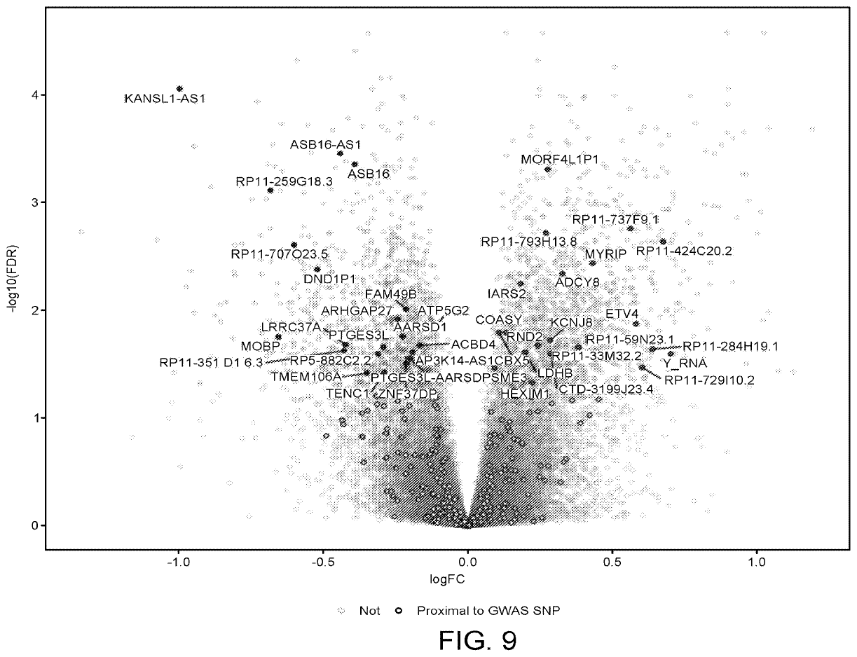

One of the top perturbed genes was the antisense RNA KANSL1-AS1 which was significantly lower in PSP brains (log FC=−1.00, FDR=8.790E-5, FIGS. 1D-1E and 9, Table 8). Both KANSL1-AS1 and its sense gene KANSL1 were located within the PSP risk genome-wide association study (GWAS) locus near tau-encoding MAPT. Another top perturbed gene, astrocyte-enriched YAP1, which was significantly higher in PSP brains, was identified as a regulatory network hub gene that was higher in AD brains.

TABLE 8
PSP DEGs that are within 1 Mbp of a PSP GWAS locus.

| DEG | | |
|---|---|---|
| Direction | GeneName | GWAS SNP |
| Down in | AARSD1 | rs8070723(MAPT), |
| PSP | ASB16 | rs242557(MAPT) |
| | ASB16-AS1 | |
| | MOBP | rs10675541(MOBP), |
| | | rs1768208(MOBP) |
| | PTGES3L | rs8070723(MAPT), |
| | PTGES3L- | rs242557(MAPT) |
| | AARSD1 | |
| | RP5-882C2.2 | |
| | TMEM106A | |
| | ACBD4 | rs71920662(MAPT) |
| | ARHGAP27 | |
| | ATP5G2 | rs147124286(SP1) |
| | DND1P1 | rs71920662(MAPT) |
| | FAM49B | rs2045091(ASAP1) |
| | KANSL1- | rs71920662(MAPT) |
| | AS1 | |
| | LRRC37A | |
| | MAP3K14- | |
| | AS1 | |
| | RP11- | |
| | 259G18.3 | |
| | RP11- | rs2142991(BMS1) |
| | 351D16.3 | |
| | RP11- | rs71920662(MAPT) |
| | 707023.5 | |
| | TENC1 | rs147124286(SP1) |
| | ZNF37BP | rs2142991(BMS1) |
| Up in | COASY | rs8070723(MAPT), |
| PSP | CTD- | rs242557(MAPT) |
| | 3199123.4 | |
| | ETV4 | |
| | KCNJ8 | rs7966334(SLCO1A2), |
| | LDHB | rs11568563(SLCO1A2) |
| | MYRIP | rs10675541(MOBP), |
| | | rs1768208(MOBP) |
| | PSME3 | rs8070723(MAPT), |
| | RND2 | rs242557(MAPT) |
| | RP11- | rs7966334(SLCO1A2), |
| | 284H19.1 | rs11568563(SLCO1A2) |
| | RP11- | |
| | 424C20.2 | |
| | RP11- | |
| | 59N23.1 | |
| | RP11- | |
| | 729110.2 | |
| | ADCY8 | rs2045091(ASAP1) |
| | CBX5 | rs147124286(SP1) |
| | HEXIM1 | rs71920662(MAPT) |
| | IARS2 | rs6687758(None) |
| | MORF4L1P1 | |
| | RP11- | rs57113693(STX6) |
| | 33M22.2 | |
| | RP11- | rs2045091(ASAP1) |
| | 737F9.1 | |
| | RP11- | rs147124286(SP1) |
| | 793H13.8 | |

Glial Cell-Enriched Gene Co-Expression Network Modules are Associated with PSP

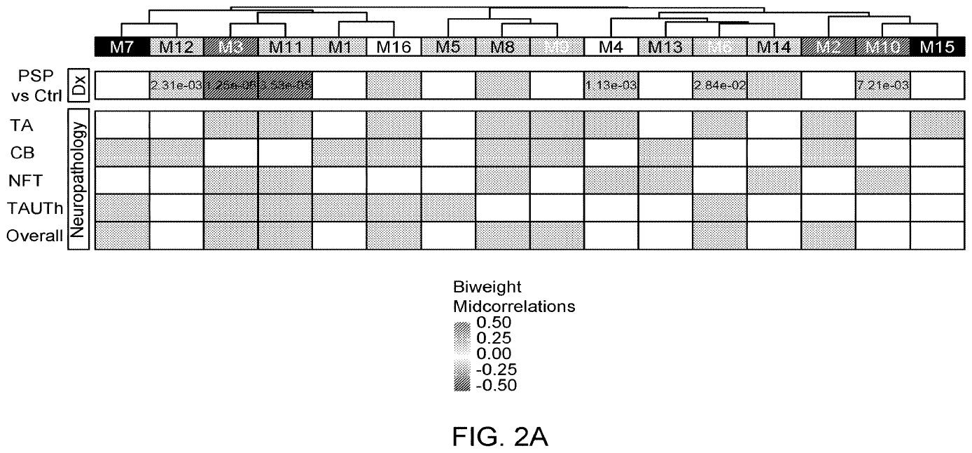

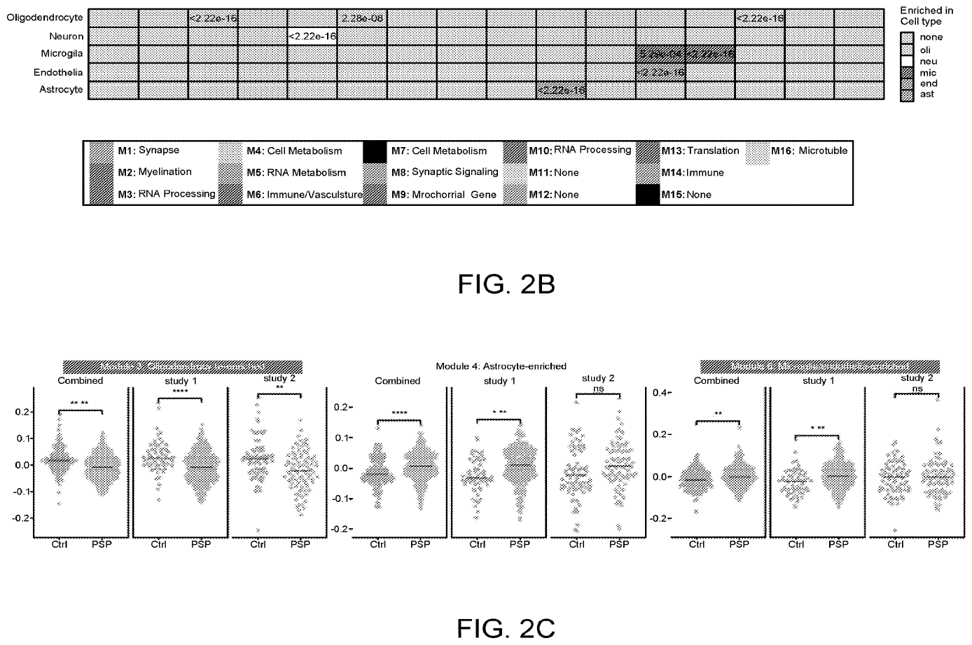

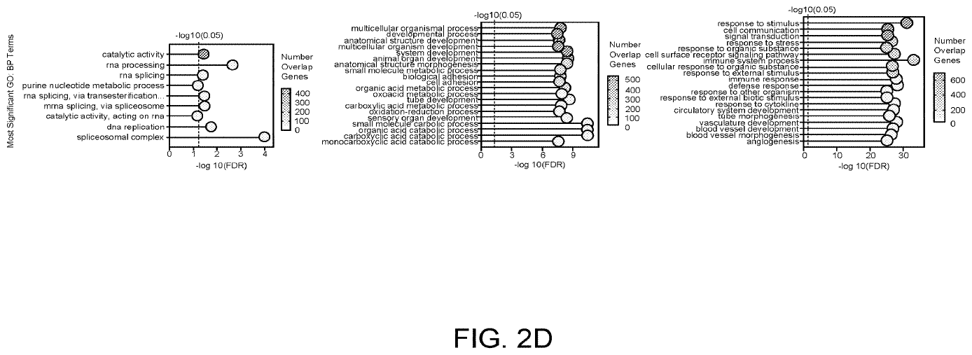

To identify and characterize groups of co-expressed genes associated with PSP, harmonized weighted gene co-expression network analysis (WGCNA) was performed across both studies. A total of 16 modules were detected using the consensus network construction algorithm after harmonized gene assignments (FIGS. 2A-2D). These consensus network modules were robust with high agreement between the gene expression clusters and module assignments (FIG. 10A) and high preservation of modules between the two studies (FIG. 10B). The ME, a summary measure of all gene expression values in the module, were correlated with PSP diagnosis and each neuropathology phenotype (FIG. 2A). Six modules that were significantly (Bonferroni adjusted p<0.05) associated with PSP diagnosis were identified. Among them, 3 modules had higher expression in PSP cases (M4, M6, and M12) and 3 modules had lower expression in PSP cases (M3, M10, M11). These associations were consistent across both studies (FIG. 11).

It was evaluated whether these modules represent gene expression perturbations in distinct cell types and biological pathways (FIGS. 2B-2D). Indeed, three of the PSP-associated modules were enriched for glial genes. An enrichment of oligodendrocyte genes (Bonferroni-adjusted p<2.22E-16) in the down-regulated M3 was found. M6, which was up-regulated in PSP, was enriched for endothelial (Bonferroni-adjusted p<2.22E-16) and microglial (Bonferroni-adjusted p=5.29E-4) marker genes, whereas M4, also up-regulated in PSP, was an astrocyte-enriched module (Bonferroni-adjusted p<2.22E-16). GO terms enriched in co-expression modules were broadly consistent with their cell types (FIGS. 2D and 12). Microglial/endothelial M6 was enriched for immunity- and vascular-related GO terms, while astrocytic M4 had metabolic, and oligodendroglial M3 had RNA processing/splicing GO term enrichment. These findings suggest that cell-type specific transcriptional changes in PSP were indicators of disrupted biological processes including immune activation, angiogenesis, cell energetics, and RNA metabolism.

### Single-Nucleus RNAseq Captures Glial Expression Changes in PSP Brains

Studies used to identify glial gene expression changes in bulk brain RNAseq were replicated in two independent studies by conducting snRNAseq experiments in brain tissue samples from the TCX of PSP and control patients (Table 5). After QC, a total of 26,241 nuclei were obtained from 34 brain samples comprising 18 PSP and 16 controls. To ensure cell populations could be reliably identified across each sample, the snRNAseq data, treating each sample as a batch variable, were integrated. Nuclei were clustered based on the integrated PCA embedding using the Louvain algorithm implemented in Seurat, which yielded 28 nuclei clusters. (FIG. 3A). There was no statistically significant enrichment of nuclei from either sex or diagnosis in any cluster (FIGS. 13A-13C). Although a few samples were statistically over-represented in 8 clusters, all except one of these (cluster 12=CL12) contributed to <1% of the total nuclei, indicating homogeneity for many of the nuclear clusters with respect to sex, diagnosis, and samples.

Each cluster was annotated based on the overlap of overexpressed cluster-marker genes and known cell-type marker genes curated manually (FIG. 3B) or via published databases (FIGS. 14-15). All major brain cell types were identified (FIG. 3C). All clusters could be annotated according to their cell type except three small clusters (CL23, CL24, and CL26), which constitute<1% of total nuclei.

Upon comparison of the expression levels between PSP and control nuclei in each cluster, DEGs at FDR<0.05 for genes expressed in >10% of the nuclei in the cluster analyzed were identified. DEGs that were up- or down-regulated in neuronal and glial clusters (Table 9) were detected. Next, the genes from the three glial-marker enriched WGCNA co-expression modules that were replicably associated with PSP in bulk RNAseq from two studies (M3, M4, and M6) were analyzed. Expression scores, calculated based on the average expression levels of these modules' genes for each nucleus in the snRNAseq, were analyzed across clusters for each cell type (FIG. 3D). Module genes had high expression in the nuclei corresponding to the enriched cell type of each module. Oligodendrocyte-enriched module M3, astrocyte-enriched M4, and microglia/endothelia-enriched M6 genes had the highest expression in oligodendrocyte, astrocyte, and endothelia snRNAseq clusters, respectively, thus validating module cell-type annotations. There was expression of these module genes in other cellular clusters as well, underscoring that they were enriched in but not exclusive to specific cell types.

TABLE 9
Number of DEG (PSP vs Ctrl) in each snRNAseq cluster.

| Cell Type | Cluster | Up | Down | N.S. |
|---|---|---|---|---|
| Astrocyte | CL2 | 1477 | 1121 | 28240 |
| Endothelia | CL19 | 1 | 4 | 30833 |
| Excitatory neuron | CL22 | 34 | 14 | 30790 |
| | CL21 | 23 | 14 | 30801 |
| | CL20 | 20 | 5 | 30813 |
| | CL18 | 282 | 78 | 30478 |
| | CL15 | 99 | 21 | 30718 |
| | CL14 | 104 | 71 | 30663 |
| | CL9 | 1946 | 534 | 28358 |
| | CL8 | 1451 | 725 | 28662 |
| | CL1 | 1354 | 2260 | 27224 |
| Inhibitory neuron | CL16 | 12 | 19 | 30807 |
| | CL13 | 133 | 260 | 30445 |
| | CL11 | 47 | 41 | 30750 |
| | CL10 | 40 | 60 | 30738 |
| | CL6 | 403 | 484 | 29951 |
| | CL5 | 658 | 996 | 29184 |
| | CL4 | 270 | 638 | 29930 |
| Microglia | CL27 | 0 | 0 | 30838 |
| | CL3 | 537 | 659 | 29642 |
| Neuron | CL24 | 22 | 37 | 30779 |
| | CL23 | 0 | 0 | 30838 |
| None | CL26 | 0 | 0 | 30838 |
| Oligodendrocyte | CL25 | 1 | 0 | 30837 |
| | CL0 | 1293 | 1242 | 28303 |
| OPC | CL7 | 440 | 412 | 29986 |
| Pericyte | CL17 | 5 | 4 | 30829 |
| | CL12 | 44 | 35 | 30759 |

DEGs are defined as genes that are significantly (FDR-adjusted p value < 0.05) up-regulated (logFC > 0) or down-regulated (logFC < 0) in PSP compared to control, AND the gene is expressed in at least 10% of the nuclei in the cluster

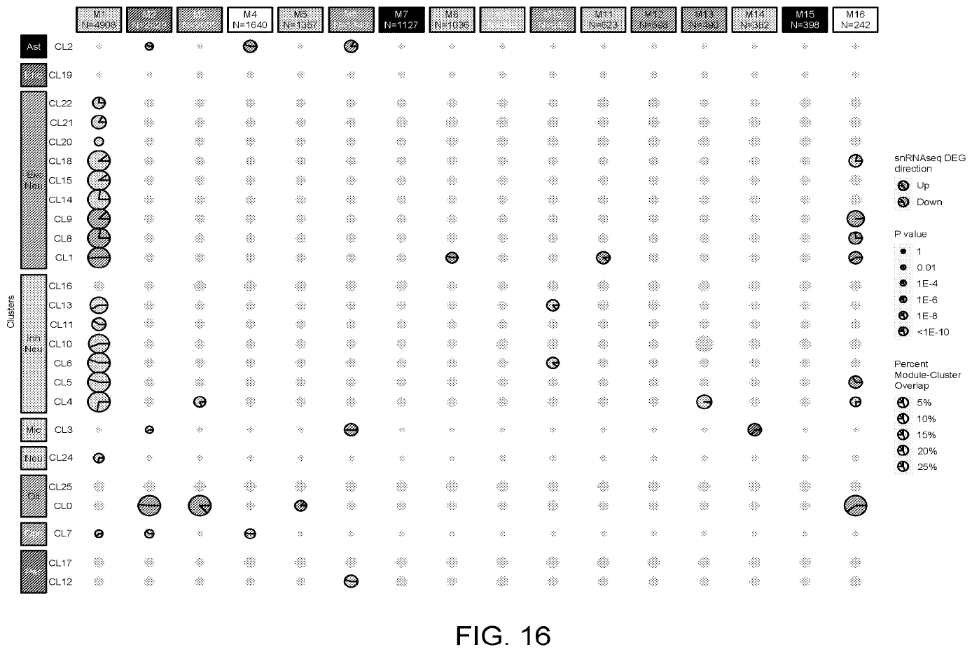

The overlap between the snRNAseq DEGs in each nuclear cluster with genes from the three modules (FIGS. 3E and 16) was evaluated. Oligodendrocyte-enriched M3 genes have the most significant overlap (p=2.74E-10) with DEGs of the oligodendrocyte cluster CLO. Further, 86.76% of these overlapping snRNAseq genes were down in PSP, consistent with the negative association between M3 eigengene and PSP (FIG. 2A). Astrocyte-enriched M4 genes overlap mostly with astrocyte CL2 DEGs (p=4.48E-49). These overlapping genes have a slightly higher proportion of up DEGs (54.52%), consistent with the positive association between M4 eigengene and PSP (FIG. 2A). Microglia/endothelia-enriched M6 genes significantly overlap with microglia CL3 DEGs (p=2.34E-10), pericytes CL12 DEGs (p=3.90E-4) and astrocytes CL2 DEGs (p=3.35E-20). The direction of change was mostly up in astrocytes (82.36% up) and mixed in the other clusters (microglia: 49.57% up, pericytes: 53.85% up).

In summary, snRNAseq data corroborated the cell-enrichment annotations for the PSP-associated bulk RNAseq modules, M3, M4, and M6. Directionality of gene expression changes for these modules' genes was consistent between bulk RNAseq and snRNAseq oligodendrocyte clusters, although other glial clusters had DEGs that changed in both directions, suggesting that single nucleus data captured subtle gene expression changes.

### Prioritization of Glial Gene Expression Changes in PSP

The bulk RNAseq data analyses yielded expression perturbations within glial cell-enriched co-expression modules (M3, M4, and M6) comprising a high number of genes (n=4,969). The subset of these module genes that were also significant snRNAseq DEGs in the same glial cell clusters still constituted a large number for experimental validations (FIG. 16). A systematic data-driven prioritization approach was applied to further narrow down and select genes using the human brain transcriptome data from this study and from a study using a tau mouse model described elsewhere (see, for example, Ramsden et al., J. Neurosci., 25:10637-10647 (2005)).

A prioritization approach is shown in FIG. 4A. Among the 4,969 genes from modules M3, M4 and M6, those that were central and highly connected within each module, defined as hub genes with module membership (MM)>0.7, were focused on. There were 550 hub genes that were also DEGs (FDR<0.05) based on the meta-analysis of two bulk brain RNAseq studies. These genes were further filtered by selecting those that were also significant DEGs in the snRNAseq data cluster corresponding to the bulk module cell-type and that had concordant direction of change between bulk and snRNAseq. For oligodendrocyte-enriched M3 and astrocyte-enriched M4 hub genes, snRNAseq DEGs that are down in PSP in oligodendrocyte and up in astrocyte clusters, respectively, were filtered for resulting in 56 and 59 genes, respectively (FIG. 4A). For the microglia/endothelia-enriched M6 hub genes, given the enrichment of snRNAseq DEGs from this module genes within these cell types (FIG. 3E), those that are up-regulated snRNAseq in microglia, endothelia, pericytes or astrocytes, were selected resulting in 40 genes. In total, there were 155 such genes which reflect robust glial gene expression changes in PSP brains.

To determine the gene expression changes that were preserved in a mouse model of tauopathy, brain transcriptome data from a mouse model that overexpresses a mutant form of human tau encoding MAPT and develops tau neuropathology by 4 months of age was used (Ramsden et al., J. Neurosci., 25:10637-10647 (2005)). Brain gene expression data from 24 transgenic (rTg4510) mice and their wild-type control littermates sacrificed at 4.5 or 6 months of age (Table 6) were analyzed.

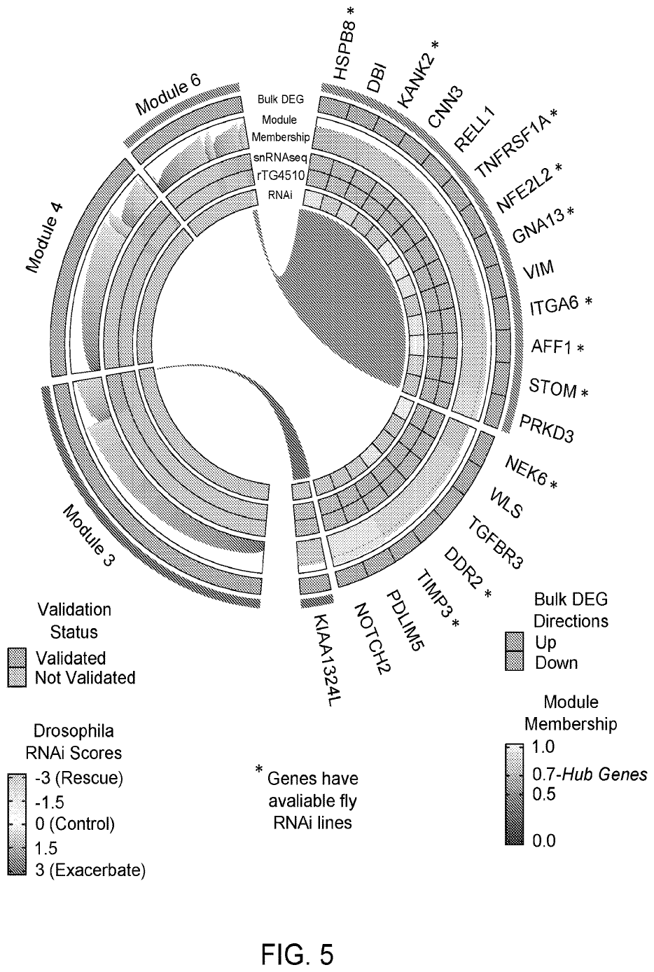

Among the 155 genes with robust glial expression changes in PSP brains, 21 were also significant DEGs perturbed in the same direction as humans in either 4.5- or 6-month rTg4510 mouse brains (FIGS. 4A-4B and 5). The rTg4510-validated genes were significantly over-represented in the astrocyte-enriched module M4 (p=1.97E-3) and microglia/endothelia-enriched module M6 (p=4.63E-18), whereas one DEG from the oligodendrocyte-enriched M3 was validated in the mouse data. The cell-type-specific overrepresentation of validated genes suggested that the rTg4510 mouse model may recapitulate tauopathy-related glial expression changes in astrocytes, endothelia, and microglia, but not in oligodendrocytes. The 21 genes that passed through the human and mouse model filters represented high confidence PSP glial DEGs with cross-species validation.

### In Vivo Experimental Validation of High Confidence PSP Glial Expression Changes

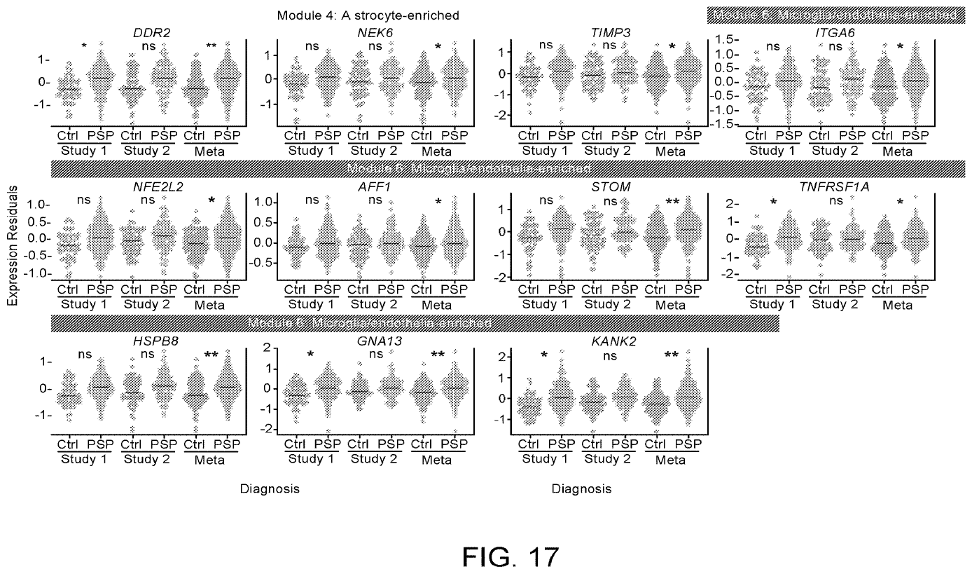

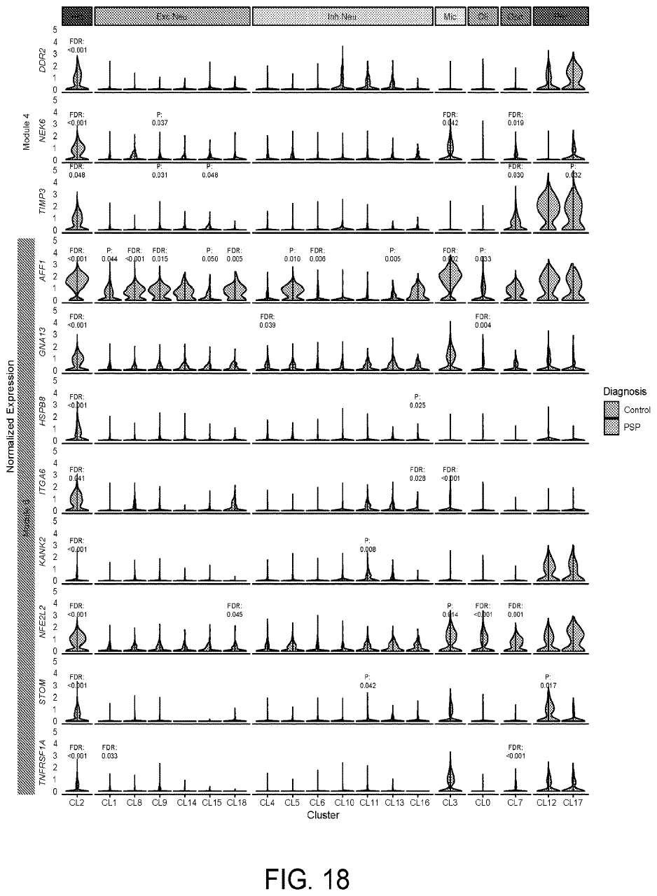

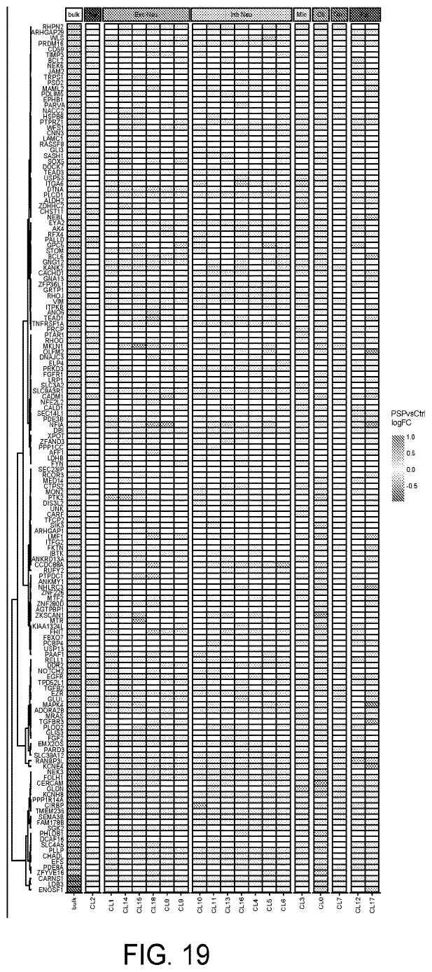

To determine whether experimentally perturbing levels of high confidence PSP glial genes have an impact on tau-related neurodegeneration, a Drosophila tau model was used (Wittmann et al., Science, 293:711-714 (2001)). Of the 21 high confidence PSP glial DEGs, 11 had an available Drosophila ortholog (FIGS. 4A-4B). Since all 11 genes were up-regulated in human PSP (FIGS. 5 and 17-18) and mouse brain transcriptome, it was evaluated whether down-regulation of these genes with RNAi in the Drosophila tau model would ameliorate neurodegeneration, as measured by eye morphology.

Using a semi-quantitative scoring system, where negative scores reflect suppression and positive scores reflect enhancement of the neurodegenerative eye morphology, it was determined that RNAi suppression of 10 out of the 11 tested genes suppressed the neurodegenerative eye morphology in Drosophila tau model (FIGS. 4C-4D). Three of these genes, DDR2, KANK2 and STOM, had the strongest suppressive effect on Drosophila tau eye phenotype (Score<−1.5), when downregulated with RNAi.

DDR2 was a hub gene in the astrocyte-enriched M4, whereas KANK2 and STOM were hubs in the microglia/endothelia-enriched M6 (FIGS. 4E-4F, 5, and 17-18). In alignment with the prioritization paradigm, all three genes were upregulated (FDR<0.05) in PSP brains based on bulk RNAseq (FIG. 4E), in snRNAseq (astrocyte cluster, FIG. 4F), and tau mouse model rTg4510 brain transcriptome (FIG. 5).

Together, these results demonstrate that upregulation of DDR2, KANK2, and/or STOM can be a pathogenic event in PSP, and that one or more DDR2 inhibitors, one or more KANK2 inhibitors, and/or one or more STOM inhibitors can be used to ameliorate tau-related neurodegeneration.

### Example 2: Inhibiting DDR2, KANK2, and/or STOM

### RNAi Knockdown of DDR2, KANK2, and STOM in Drosophila

To assess the impact of the three top tau-toxicity suppressor genes, crosses were set up between female GMR-GAL4/CyO; UAS-hTau/TM3 flies and male flies with RNAi flies from Bloomington Drosophila Stock Center (stock IDs: 33432, 55906, 65975). The progenies that co-expresses tau and RNAi were selected and aged for 5 days at 25° C. before pictures of the fly left eyes were taken. The severity of the tau-induced eye degeneration was assessed blindly based on the following categories: loss of bristle (0-1), size (0-1), color (0-2), the presence of necrotic patterns (0-2), the collapse of the eye (0-2), and the loss of ommatidia (0-2), where a higher score indicates a more severe phenotype. Scores were provided by two independent evaluators separately, and the average scores were used for analysis.

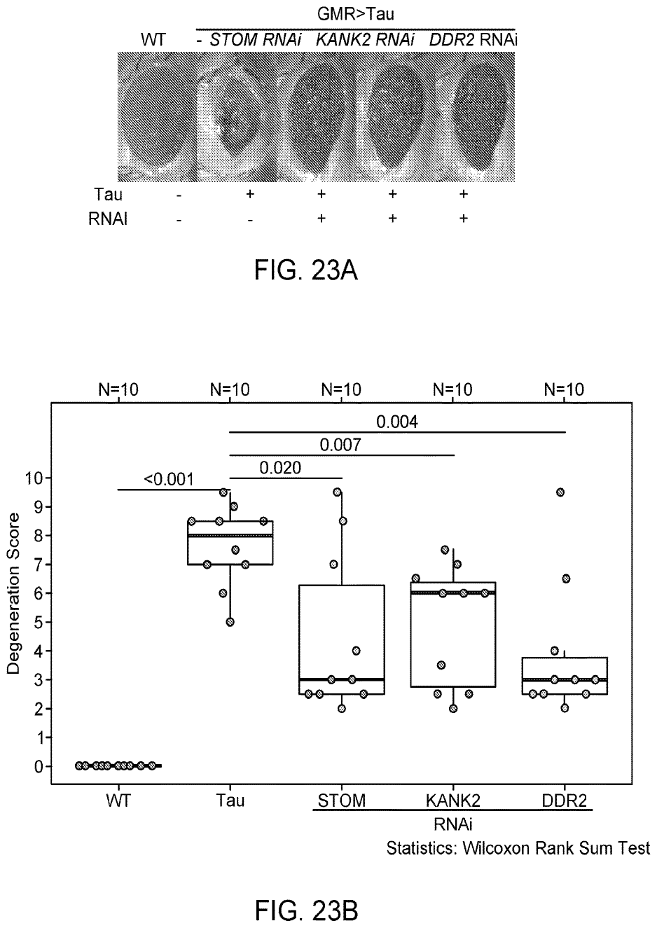

A robust rescue of tau-mediated toxicity was observed when inhibiting the expression of the fly ortholog of STOM, KANK2, or DDR2 with RNAi (FIG. 23A). In addition, a significant reduction in eye degeneration was observed (FIG. 23B).

### Small Molecule Inhibitor of DDR2 in Drosophila

To assess the impact of DDR2 inhibitor on tau mediated cell toxicity were assessed using the GMR>Tau system as described in Example 1. Crosses were set up with regular fly food supplemented with either dasatinib (final concentrations of 1 μM or 10 μM) or DMSO (0.5%). Progenies with the correct genotypes were transferred to fresh dasatinib- or DMSO-containing food after eclosing, and aged for 5 days, changing to fresh dasatinib- or DMSO-containing food every 2 days. The fly eye degeneration was assessed with blinded evaluators using the same scale as described earlier in the document.

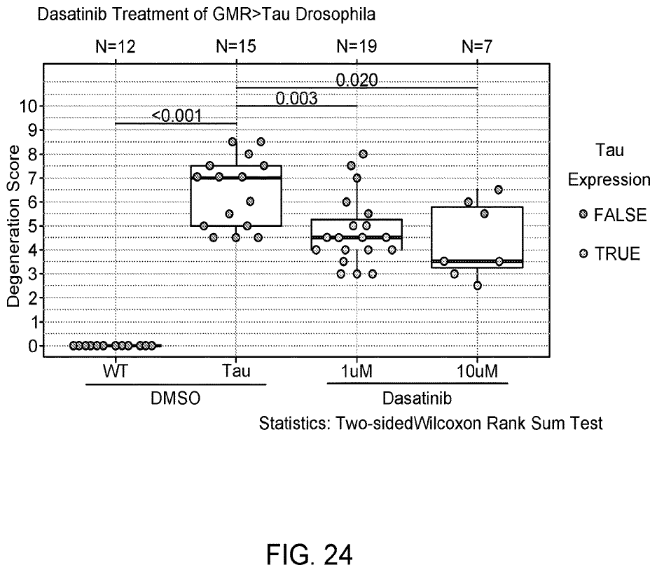

Treatment of DDR2 inhibitor dasatinib created a dosage-dependent reduction in tau-mediated eye degeneration in Drosophila (FIG. 24).

### Example 3: Exemplary Anti-Sense Oligonucleotides

### Exemplary Modifications

| Key | Modification | Category |
|---|---|---|
| /52MOErA/ | 5′ 2′-O-methoxyethyl A | Modified Base |
| /52MOErG/ | 5′ 2′-O-methoxyethyl G | Modified Base |
| /52MOErC/ | 5′ 2′-O-methoxyethyl MeC | Modified Base |
| /52MOErT/ | 5′ 2′-O-methoxyethyl T | Modified Base |
| /i2MOErA/ | internal 2′-O-methoxyethyl A | Modified Base |
| /i2MOErG/ | internal 2′-O-methoxyethyl G | Modified Base |
| /i2MOErC/ | internal 2′-O-methoxyethyl MeC | Modified Base |
| /i2MOErT/ | internal 2′-O-methoxyethyl T | Modified Base |
| /32MOErA/ | 3′ 2′-O-methoxyethyl A | Modified Base |
| /32MOErG/ | 3′ 2′-O-methoxyethyl G | Modified Base |
| /32MOErC/ | 3′ 2′-O-methoxyethyl MeC | Modified Base |
| /32MOErT/ | 3′ 2′-O-methoxyethyl T | Modified Base |
| * | Phosphorothioate bond | Modified Linkage |

Exemplary ASOs designed to induce RNAi of DDR2 polypeptide expression containing one or more modifications:

| SEQ ID No: | |
|---|---|
| containing | |
| exemplary | |
| modifications | Sequence containing exemplary modifications |
| 1 | /52MOErC/*/i2MOErA/*/i2MOErG/*/i2MOErA/*/i2MOErA/*C*T*C*A*A*G*A* |
| | T*A*G*/i2MOErG/*/i2MOErC/*/i2MOErA/*/i2MOErG/*/32MOErC/ |
| 2 | /52MOErG/*/i2MOErT/*/i2MOErG/*/i2MOErA/*/i2MOErA/*A*T*T*C*C*T*G* |
| | A*T*G*/i2MOErC/*/i2MOErG/*/i2MOErG/*/i2MOErT/*/32MOErC/ |
| 3 | /52MOErG/*/i2MOErA/*/i2MOErA/*/i2MOErG/*/i2MOErT/*A*G*C*A*C*T*G* |
| | T*A*C*/i2MOErC/*/i2MOErT/*/i2MOErC/*/i2MOErC/*/32MOErT/ |
| 4 | /52MOErA/*/i2MOErC/*/i2MOErA/*/i2MOErG/*/i2MOErA/*G*T*C*A*G*G*A* |
| | C*A*A*/i2MOErA/*/i2MOErT/*/i2MOErG/*/i2MOErG/*/32MOErC/ |
| 5 | /52MOErC/*/i2MOErA/*/i2MOErC/*/i2MOErT/*/i2MOErC/*T*T*G*C*A*G*C* |
| | A*G*A*/i2MOErC/*/i2MOErA/*/i2MOErT/*/i2MOErT/*/32MOErC/ |
| 6 | /52MOErA/*/i2MOErG/*/i2MOErA/*/i2MOErA/*/i2MOErT/*C*A*A*G*G*A*C* |
| | T*C*T*/i2MOErC/*/i2MOErG/*/i2MOErG/*/i2MOErA/*/32MOErG/ |
| 7 | /52MOErA/*/i2MOErG/*/i2MOErG/*/i2MOErT/*/i2MOErA/*A*A*C*T*G*G*T* |
| | G*A*G*/i2MOErG/*/i2MOErG/*/i2MOErC/*/i2MOErA/*/32MOErA/ |
| 8 | /52MOErA/*/i2MOErA/*/i2MOErG/*/i2MOErG/*/i2MOErT/*A*C*T*T*C*A*T* |
| | G*C*C*/i2MOErA/*/i2MOErG/*/i2MOErA/*/i2MOErG/*/32MOErG/ |
| 9 | /52MOErC/*/i2MOET/*/i2MOErG/*/i2MOErC/*/i2MOErT/*C*A*T*T*C*C*A* |
| | A*A*G*/i2MOErT/*/i2MOErC/*/i2MOErA/*/i2MOErG/*/32MOErC/ |
| 10 | /52MOErG/*/i2MOErC/*/i2MOErA/*/i2MOErC/*/i2MOErT/*G*A*C*A*T*C*T* |
| | A*G*G*/i2MOErG/*/i2MOErC/*/i2MOErA/*/i2MOErA/*/32MOErA/ |

Exemplary ASOs designed to induce RNAi of KANK2 polypeptide expression containing one or more modifications:

| SEQ ID No: | |
|---|---|
| containing | |
| exemplary | |
| modifications | Sequence containing exemplary modifications |
| 17 | /52MOErT/*/i2MOErC/*/i2MOErT/*/i2MOErC/*/i2MOErC/*A*G*A*G*C*A*C* |
| | C*A*T*/i2MOErA/*/i2MOErC/*/i2MOErT/*/i2MOErG/*/32MOErA/ |
| 18 | /52MOErC/*/i2MOErC/*/i2MOErT/*/i2MOErC/*/i2MOErC/*G*T*G*T*T*T*G* |
| | A*T*C*/i2MOErC/*/i2MOErT/*/i2MOErG/*/i2MOErA/*/32MOErT/ |
| 19 | /52MOErC/*/i2MOErA/*/i2MOErC/*/i2MOET/*/i2MOErT/*G*A*T*G*T*T*C* |
| | A*T*G*/i2MOErC/*/i2MOErG/*/i2MOErG/*/i2MOErG/*/32MOErA/ |
| 20 | /52MOErG/*/i2MOErC/*/i2MOErA/*/i2MOErA/*/i2MOErG/*C*C*A*G*T*C*T* |
| | A*C*A*/i2MOErT/*/i2MOErC/*/i2MOErA/*/i2MOErA/*/32MOErG/ |
| 21 | /52MOErC/*/i2MOErC/*/i2MOErC/*/i2MOErT/*/i2MOErC/*A*G*A*C*A*C*C* |
| | C*A*C*/i2MOErA/*/i2MOErA/*/i2MOErT/*/i2MOErT/*/32MOErA/ |
| 22 | /52MOErC/*/i2MOErG/*/i2MOErT/*/i2MOErA/*/i2MOErC/*C*G*T*G*A*T*C* |
| | A*G*A*/i2MOErA/*/i2MOErA/*/i2MOErG/*/i2MOErT/*/32MOErG/ |
| 23 | /52MOErC/*/i2MOErC/*/i2MOErC/*/i2MOErT/*/i2MOErG/*T*C*T*C*T*T*G*A |
| | *A*G*/i2MOErT/*/i2MOErT/*/i2MOErT/*/i2MOErG/*/32MOErC/ |
| 24 | /52MOErT/*/i2MOErC/*/i2MOErT/*/i2MOErC/*/i2MOErT/*G*T*G*C*T*G*T*C |
| | *G*T*/i2MOErT/*/i2MOErG/*/i2MOErT/*/i2MOErC/*/32MOErT/ |
| 25 | /52MOErA/*/i2MOErT/*/i2MOErC/*/i2MOErC/*/i2MOErA/*T*C*C*A*T*C*C* |
| | A*T*C*/i2MOErC/*/i2MOErT/*/i2MOErG/*/i2MOErC/*/32MOErT/ |
| 26 | /52MOErC/*/i2MOErG/*/i2MOErT/*/i2MOErC/*/i2MOErC/*T*G*T*C*T*T*G*C |
| | *T*T*/i2MOErT/*/i2MOErG/*/i2MOErT/*/i2MOErC/*/32MOErT/ |

Exemplary ASOs designed to induce RNAi of STOM polypeptide expression containing one or more modifications:

| SEQ ID No: | |
|---|---|
| containing | |
| exemplary | |
| modifications | Sequence containing exemplary modifications |
| 27 | /52MOErG/*/i2MOErG/*/i2MOErA/*/i2MOErC/*/i2MOErT/*A*C*G*A*A*T*T*C |
| | *A*C*/i2MOErA/*/i2MOErA/*/i2MOErG/*/i2MOErG/*/32MOErC/ |
| 28 | /52MOErA/*/i2MOErC/*/i2MOErA/*/i2MOErT/*/i2MOErT/*G*T*T*G*G*A*A*A |
| | *G*G*/i2MOErG/*/i2MOErA/*/i2MOErG/*/i2MOErG/*/32MOErC/ |
| 29 | /52MOErA/*/i2MOErA/*/i2MOErC/*/i2MOErA/*/i2MOErA/*A*C*C*A*G*G*T* |
| | C*C*T*/i2MOErT/*/i2MOErT/*/i2MOErG/*/i2MOErG/*/32MOErC/ |
| 30 | /52MOErG/*/i2MOErC/*/i2MOErA/*/i2MOErA/*/i2MOET/*G*G*T*G*G*T*C*A |
| | *G*T*/i2MOErG/*/i2MOErT/*/i2MOErC/*/i2MOErT/*/32MOErG/ |
| 31 | /52MOErG/*/i2MOErG/*/i2MOErT/*/i2MOErT/*/i2MOErC/*A*G*A*A*T*G*A* |
| | G*T*C*/i2MOErA/*/i2MOErG/*/i2MOErT/*/i2MOErG/*/32MOErG/ |
| 32 | /52MOErT/*/i2MOErC/*/i2MOErT/*/i2MOErC/*/i2MOErT/*G*G*A*G*G*T*A*A |
| | *G*G*/i2MOErC/*/i2MOErA/*/i2MOErC/*/i2MOErA/*/32MOErT/ |
| 33 | /52MOErT/*/i2MOErC/*/i2MOErT/*/i2MOErG/*/i2MOErA/*A*A*C*A*C*G*G*T |
| | *C*T*/i2MOErG/*/i2MOErG/*/i2MOErA/*/i2MOErT/*/32MOErT/ |
| 34 | /52MOErA/*/i2MOErT/*/i2MOErG/*/i2MOErC/*/i2MOErC/*A*G*G*T*T*G*C*T |
| | *C*A*/i2MOErG/*/i2MOErA/*/i2MOErT/*/i2MOErT/*/32MOErC/ |
| 35 | /52MOErA/*/i2MOErG/*/i2MOErT/*/i2MOErT/*/i2MOErT/*G*A*C*A*G*T*A*T |
| | *C*T*/i2MOErG/*/i2MOErC/*/i2MOErC/*/i2MOErC/*/32MOErA/ |
| 36 | /52MOErA/*/i2MOErC/*/i2MOErA/*/i2MOErC/*/i2MOErG/*C*T*C*C*A*C*C*T |
| | *T*T*/i2MOErA/*/i2MOErT/*/i2MOErT/*/i2MOErC/*/32MOErC/ |

### Example 4: Inhibiting DDR2, KANK2, and/or STOM

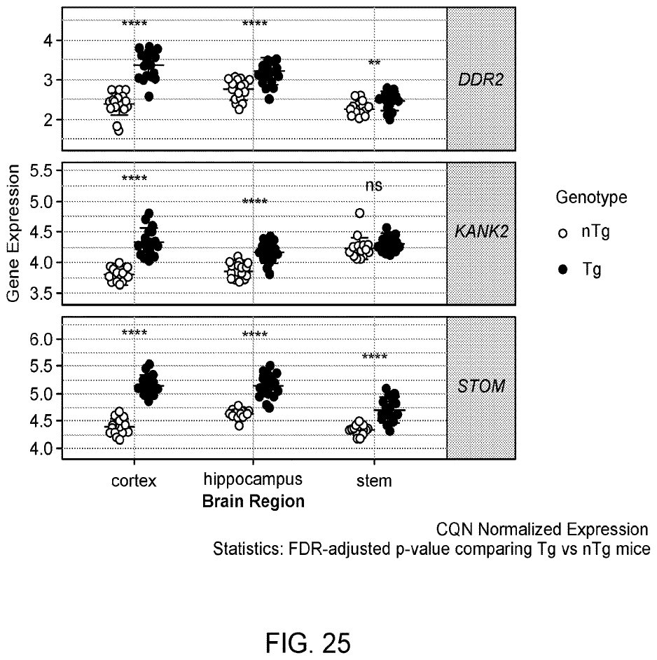

Expression of DDR2, KANK2, and STOM was assessed using Tau P301S mouse RNAseq data (Swarup et al., Nat. Med., 25 (1): 152-164 (2019)). Target genes were significantly up-regulated in the transgenic mice compared to non-transgenic control (FIG. 25).

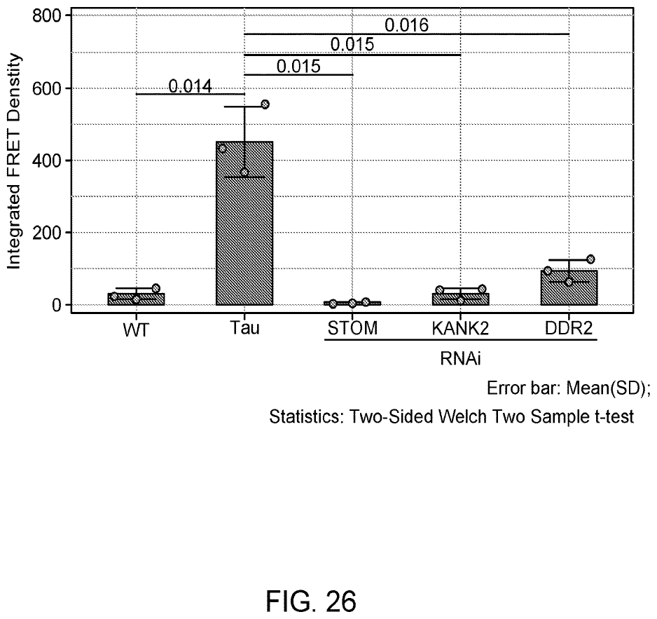

The relationship between the target genes and tau seeding activity was assessed. When DDR2, KANK2, and STOM orthologs were knocked down in drosophila using RNAi described in Example 3, a statistically significant reduction of tau seeding activity was observed in tau transgenic Drosophila brains (FIG. 26).

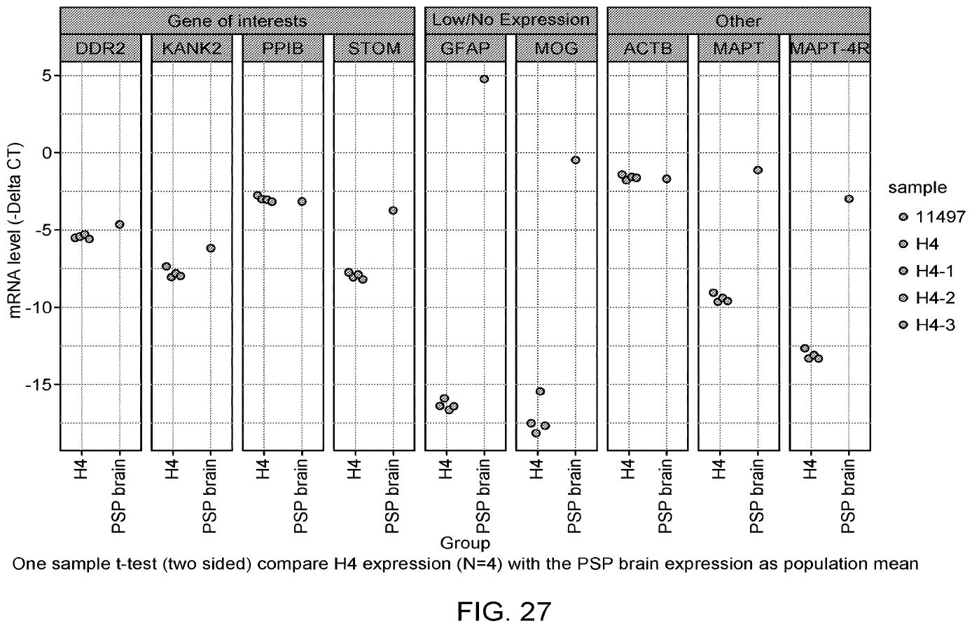

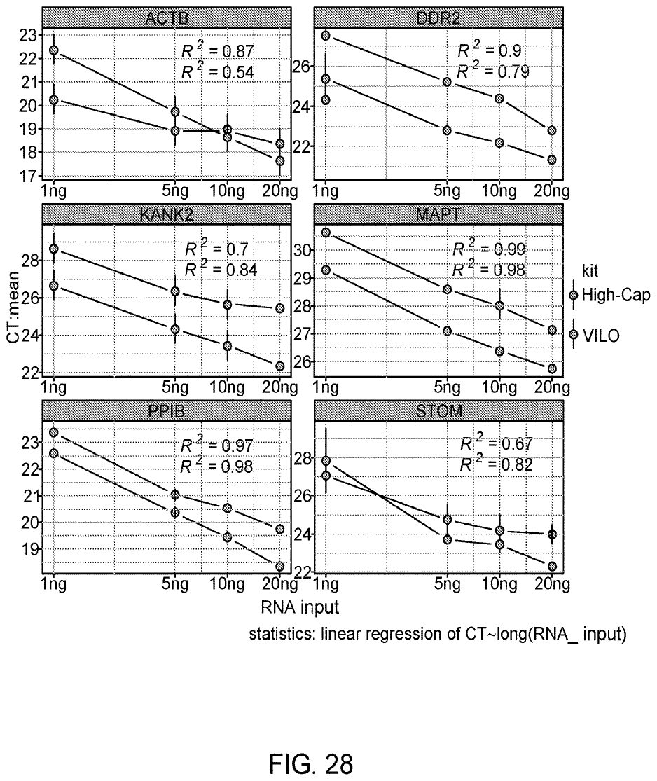

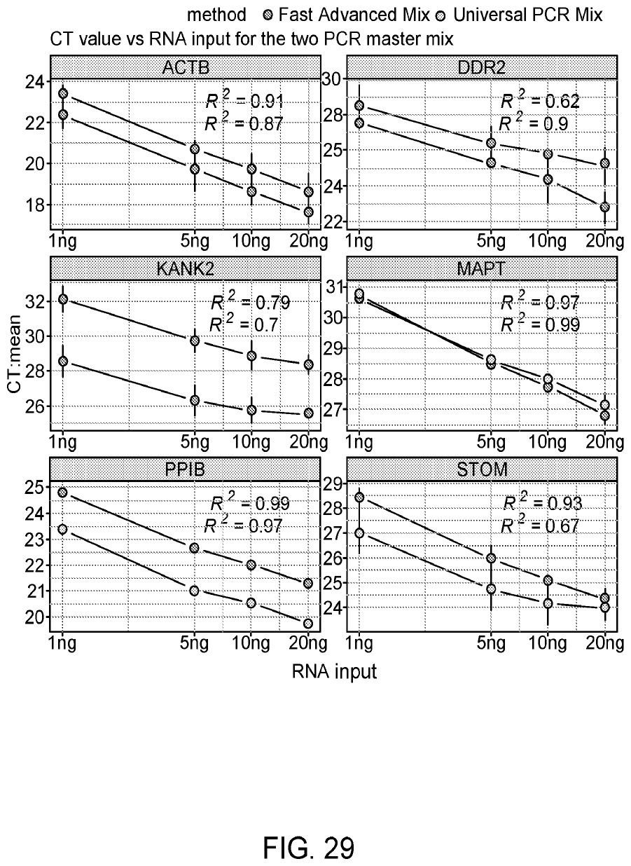

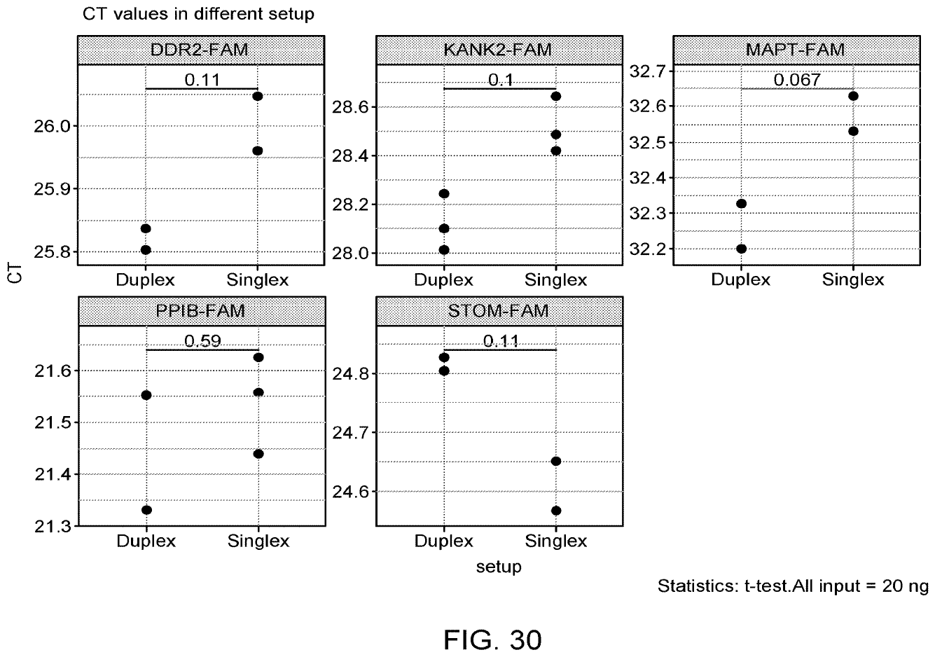

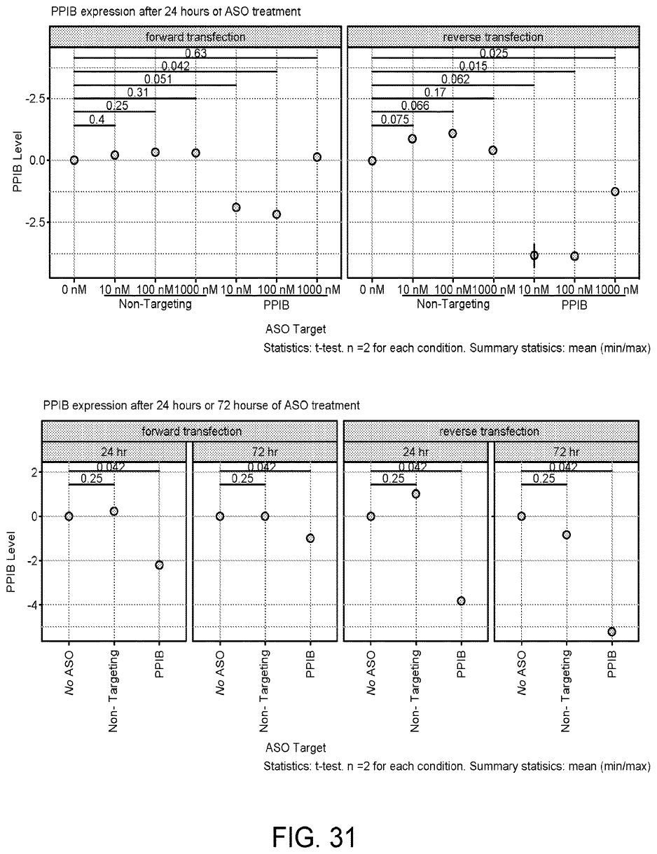

Expression levels of DDR2, KANK2, and STOM were confirmed to have similar expression levels in the H4 neuroglioma cells (ATCC cat #HTB-148) and PSP brain homogenate (FIG. 27), and then ASOs described in Example 3 were screened for various characteristics in H4 cells. cDNA reverse transcription kits High-Capacity cDNA Reverse Transcription Kit (High-cap, Thermo Fisher Scientific, cat #4368814) and SuperScript™ IV VILO™ (VILO, Thermo Fisher Scientific, cat #11756050) were assessed. Both cDNA synthesis kits tested quantitatively reverse transcribed RNA into cDNA (FIG. 28). qPCR master mixes TaqMan™ Fast Advanced (Fast Advance, Thermo Fisher Scientific, cat #4444556) and TaqMan™ Universal PCR Master Mix (Universal, Thermo Fisher Scientific, cat #4364340) were assessed. Both qPCR master mixes quantitatively amplified cDNA and generated detectable signals (FIG. 29). Multiplexed qPCR assays were also performed. Duplexed gene quantification yielded similar results as singleton qPCR setups (FIG. 30). ASOs targeting PPIB were used as a positive control to confirm delivery of the ASOs to H4 cells and establish ASO screening parameters (FIG. 31).

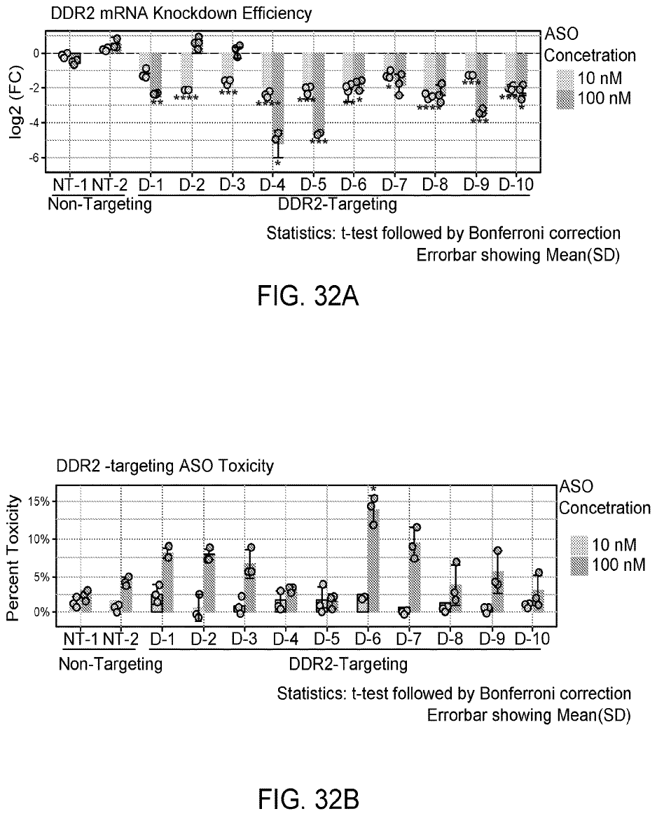

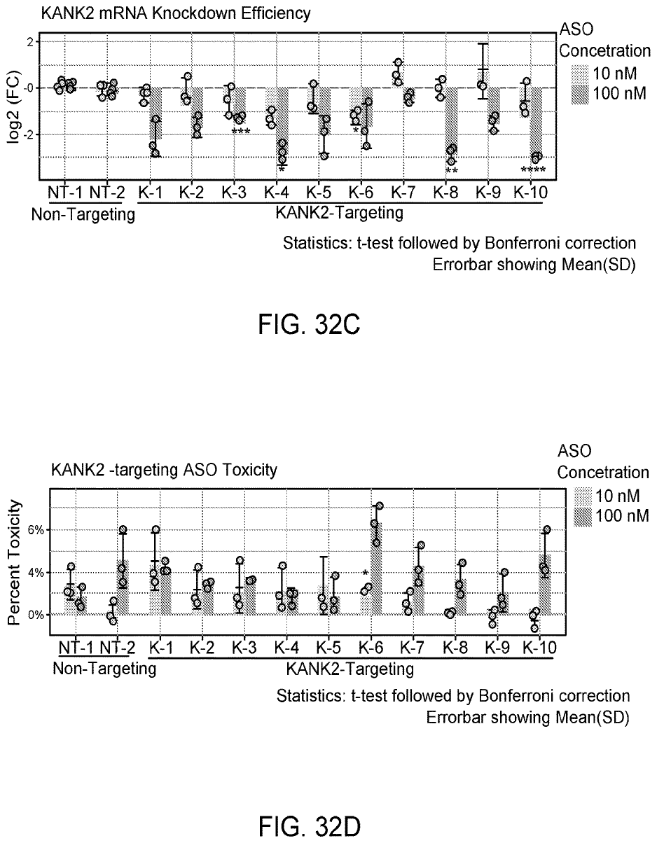

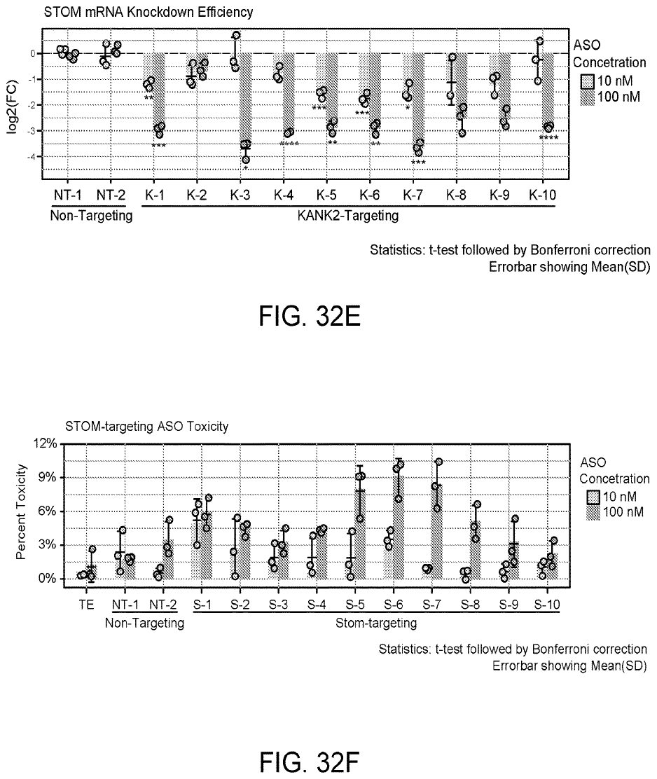

ASOs described in Example 3 were screened in the H4 neuroglioma cells. Different concentrations of ASOs (10 nM and 100 nM) were administered and knock-down efficiency (measured by fold change of mRNA compared to non-targeting ASO treated controls) and ASO toxicity (measured by LDH assay) were determined (FIGS. 32A-32F).

ASO used in this study:

| SEQ ID No: | | |
|---|---|---|
| containing | | |
| exemplary | | ASO |
| modifications | Sequence containing exemplary modifications | Label |
| 1 | /52MOErC/*/i2MOErA/*/i2MOErG/*/i2MOErA/*/i2MOErA/*C*T*C*A* | D1 |
| | A*G*A*T*A*G*/i2MOErG/*/i2MOErC/*/i2MOErA/*/i2MOErG/*/32MO | |
| | ErC/ | |
| 2 | /52MOErG/*/i2MOErT/*/i2MOErG/*/i2MOErA/*/i2MOErA/*A*T*T*C*C | D2 |
| | *T*G*A*T*G*/i2MOErC/*/i2MOErG/*/i2MOErG/*/i2MOErT/*/32MOEr | |
| | C/ | |
| 3 | /52MOErG/*/i2MOErA/*/i2MOErA/*/i2MOErG/*/i2MOErT/*A*G*C*A* | D3 |
| | C*T*G*T*A*C*/i2MOErC/*/i2MOErT/*/i2MOErC/*/i2MOErC/*/32MOE | |
| | IT/ | |
| 4 | /52MOErA/*/i2MOErC/*/i2MOErA/*/i2MOErG/*/i2MOErA/*G*T*C*A* | D4 |
| | G*G*A*C*A*A*/i2MOErA/*/i2MOErT/*/i2MOErG/*/i2MOErG/*/32MO | |
| | ErC/ | |
| 5 | /52MOErC/*/i2MOErA/*/i2MOErC/*/i2MOErT/*/i2MOErC/*T*T*G*C*A | D5 |
| | *G*C*A*G*A*/i2MOErC/*/i2MOErA/*/i2MOErT/*/i2MOErT/*/32MOEr | |
| | C/ | |
| 6 | /52MOErA/*/i2MOErG/*/i2MOErA/*/i2MOErA/*/i2MOErT/*C*A*A*G* | D6 |
| | G*A*C*T*C*T*/i2MOErC/*/i2MOErG/*/i2MOErG/*/i2MOErA/*/32MOE | |
| | rG/ | |
| 7 | /52MOErA/*/i2MOErG/*/i2MOErG/*/i2MOErT/*/i2MOErA/*A*A*C*T* | D7 |
| | G*G*T*G*A*G*/i2MOErG/*/i2MOErG/*/i2MOErC/*/i2MOErA/*/32MO | |
| | ErA/ | |
| 8 | /52MOErA/*/i2MOErA/*/i2MOErG/*/i2MOErG/*/i2MOET/*A*C*T*T*C | D8 |
| | *A*T*G*C*C*/i2MOErA/*/i2MOErG/*/i2MOErA/*/i2MOErG/*/32MOEr | |
| | G/ | |
| 9 | /52MOErC/*/i2MOET/*/i2MOErG/*/i2MOErC/*/i2MOErT/*C*A*T*T*C | D9 |
| | *C*A*A*A*G*/i2MOErT/*/i2MOErC/*/i2MOErA/*/i2MOErG/*/32MOEr | |
| | C/ | |
| 10 | /52MOErG/*/i2MOErC/*/i2MOErA/*/i2MOErC/*/i2MOErT/*G*A*C*A* | D10 |
| | T*C*T*A*G*G*/i2MOErG/*/i2MOErC/*/i2MOErA/*/i2MOErA/*/32MOE | |
| | rA/ | |
| 17 | /52MOErT/*/i2MOErC/*/i2MOET/*/i2MOErC/*/i2MOErC/*A*G*A*G*C | K1 |
| | *A*C*C*A*T*/i2MOErA/*/i2MOErC/*/i2MOErT/*/i2MOErG/*/32MOEr | |
| | A/ | |
| 18 | /52MOErC/*/i2MOErC/*/i2MOErT/*/i2MOErC/*/i2MOErC/*G*T*G*T*T | K2 |
| | *T*G*A*T*C*/i2MOErC/*/i2MOErT/*/i2MOErG/*/i2MOErA/*/32MOEr | |
| | T/ | |
| 19 | /52MOErC/*/i2MOErA/*/i2MOErC/*/i2MOErT/*/i2MOErT/*G*A*T*G*T | K3 |
| | *T*C*A*T*G*/i2MOErC/*/i2MOErG/*/i2MOErG/*/i2MOErG/*/32MOEr | |
| | A/ | |
| 20 | /52MOErG/*/i2MOErC/*/i2MOErA/*/i2MOErA/*/i2MOErG/*C*C*A*G* | K4 |
| | T*C*T*A*C*A*/i2MOErT/*/i2MOErC/*/i2MOErA/*/i2MOErA/*/32MOE | |
| | rG/ | |
| 21 | /52MOErC/*/i2MOErC/*/i2MOErC/*/i2MOErT/*/i2MOErC/*A*G*A*C* | K5 |
| | A*C*C*C*A*C*/i2MOErA/*/i2MOErA/*/i2MOErT/*/i2MOErT/*/32MOE | |
| | rA/ | |
| 22 | /52MOErC/*/i2MOErG/*/i2MOErT/*/i2MOErA/*/i2MOErC/*C*G*T*G* | K6 |
| | A*T*C*A*G*A*/i2MOErA/*/i2MOErA/*/i2MOErG/*/i2MOErT/*/32MO | |
| | ErG/ | |
| 23 | /52MOErC/*/i2MOErC/*/i2MOErC/*/i2MOErT/*/i2MOErG/*T*C*T*C*T | K7 |
| | *T*G*A*A*G*/i2MOErT/*/i2MOErT/*/i2MOErT/*/i2MOErG/*/32MOEr | |
| | C/ | |
| 24 | /52MOErT/*/i2MOErC/*/i2MOErT/*/i2MOErC/*/i2MOErT/*G*T*G*C*T | K8 |
| | *G*T*C*G*T*/i2MOErT/*/i2MOErG/*/i2MOErT/*/i2MOErC/*/32MOErT | |
| | / | |
| 25 | /52MOErA/*/i2MOErT/*/i2MOErC/*/i2MOErC/*/i2MOErA/*T*C*C*A*T | K9 |
| | *C*C*A*T*C*/i2MOErC/*/i2MOErT/*/i2MOErG/*/i2MOErC/*/32MOEr | |
| | T/ | |
| 26 | /52MOErC/*/i2MOErG/*/i2MOErT/*/i2MOErC/*/i2MOErC/*T*G*T*C*T | K10 |
| | *T*G*C*T*T*/i2MOET/*/i2MOErG/*/i2MOErT/*/i2MOErC/*/32MOErT | |
| 27 | /52MOErG/*/i2MOErG/*/i2MOErA/*/i2MOErC/*/i2MOErT/*A*C*G*A* | S1 |
| | A*T*T*C*A*C*/i2MOErA/*/i2MOErA/*/i2MOErG/*/i2MOErG/*/32MOE | |
| | rC/ | |
| 28 | /52MOErA/*/i2MOErC/*/i2MOErA/*/i2MOErT/*/i2MOErT/*G*T*T*G*G | S2 |
| | *A*A*A*G*G*/i2MOErG/*/i2MOErA/*/i2MOErG/*/i2MOErG/*/32MOEr | |
| | C/ | |
| 29 | /52MOErA/*/i2MOErA/*/i2MOErC/*/i2MOErA/*/i2MOErA/*A*C*C*A* | S3 |
| | G*G*T*C*C*T*/i2MOErT/*/i2MOErT/*/i2MOErG/*/i2MOErG/*/32MOE | |
| | rC/ | |
| 30 | /52MOErG/*/i2MOErC/*/i2MOErA/*/i2MOErA/*/i2MOErT/*G*G*T*G* | S4 |
| | G*T*C*A*G*T*/i2MOErG/*/i2MOErT/*/i2MOErC/*/i2MOErT/*/32MOE | |
| | rG/ | |
| 31 | /52MOErG/*/i2MOErG/*/i2MOErT/*/i2MOErT/*/i2MOErC/*A*G*A*A* | S5 |
| | T*G*A*G*T*C*/i2MOErA/*/i2MOErG/*/i2MOErT/*/i2MOErG/*/32MOE | |
| | rG/ | |
| 32 | /52MOErT/*/i2MOErC/*/i2MOErT/*/i2MOErC/*/i2MOET/*G*G*A*G*G | S6 |
| | *T*A*A*G*G*/i2MOErC/*/i2MOErA/*/i2MOErC/*/i2MOErA/*/32MOEr | |
| | T/ | |
| 33 | /52MOErT/*/i2MOErC/*/i2MOErT/*/i2MOErG/*/i2MOErA/*A*A*C*A* | S7 |
| | C*G*G*T*C*T*/i2MOErG/*/i2MOErG/*/i2MOErA/*/i2MOErT/*/32MOE | |
| | rT/ | |
| 34 | /52MOErA/*/i2MOErT/*/i2MOErG/*/i2MOErC/*/i2MOErC/*A*G*G*T*T | S8 |
| | *G*C*T*C*A*/i2MOErG/*/i2MOErA/*/i2MOErT/*/i2MOErT/*/32MOEr | |
| | C/ | |
| 35 | /52MOErA/*/i2MOErG/*/i2MOErT/*/i2MOErT/*/i2MOErT/*G*A*C*A* | S9 |
| | G*T*A*T*C*T*/i2MOErG/*/i2MOErC/*/i2MOErC/*/i2MOErC/*/32MOE | |
| | rA/ | |
| 36 | /52MOErA/*/i2MOErC/*/i2MOErA/*/i2MOErC/*/i2MOErG/*C*T*C*C* | S10 |
| | A*C*C*T*T*T*/i2MOErA/*/i2MOErT/*/i2MOErT/*/i2MOErC/*/32MOEr | |
| | C/ | |

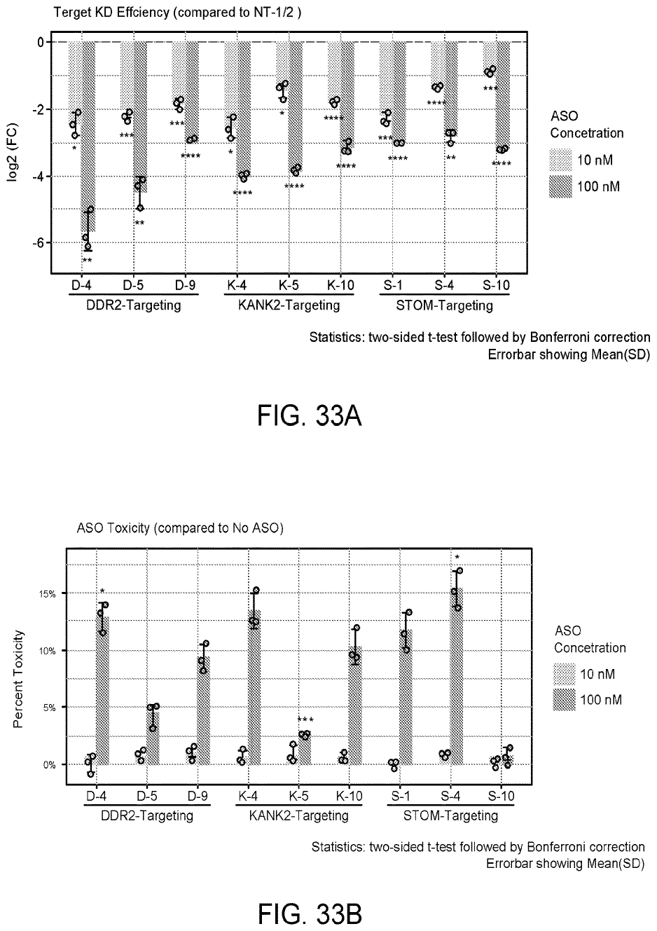

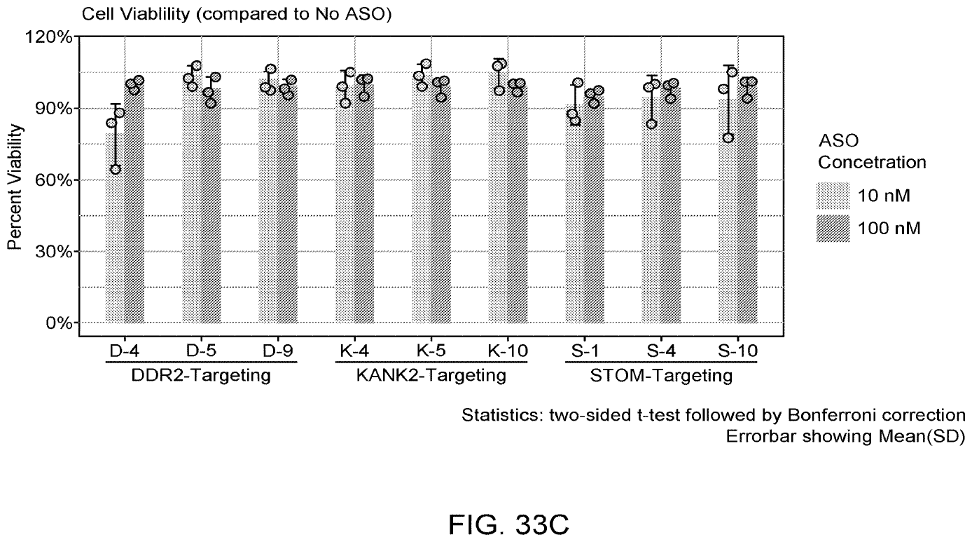

Three ASO hits were selected for each target (DDR2: D4, D5, D9; KANK2: K4, K5, K10; STOM: S1, S4, S10). The knock-down efficiency (mRNA change) and ASO safety (cell toxicity as measured by LDH assay and viability as measured by XTT assay) were repeated/performed to validate the hits, as shown in FIG. 33A-33C.

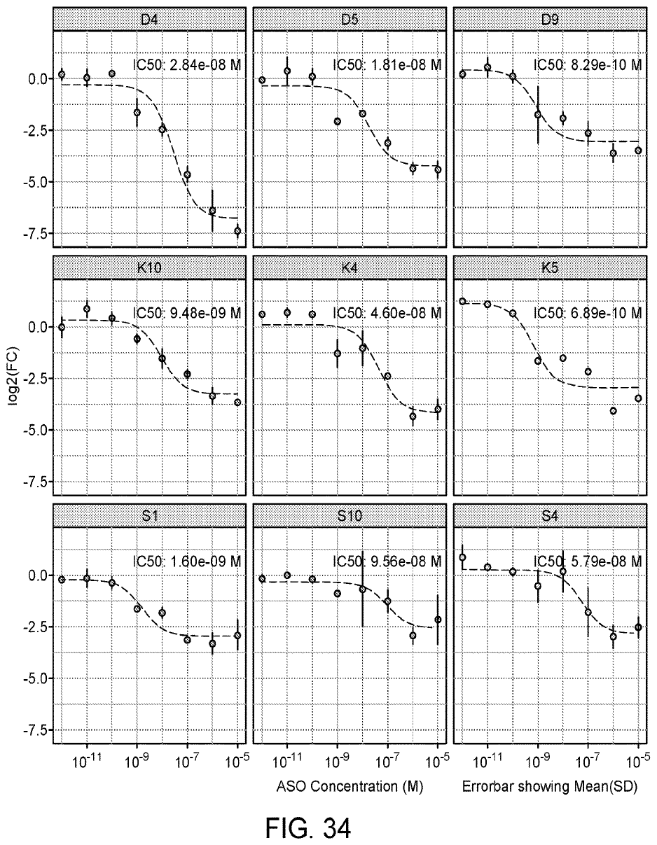

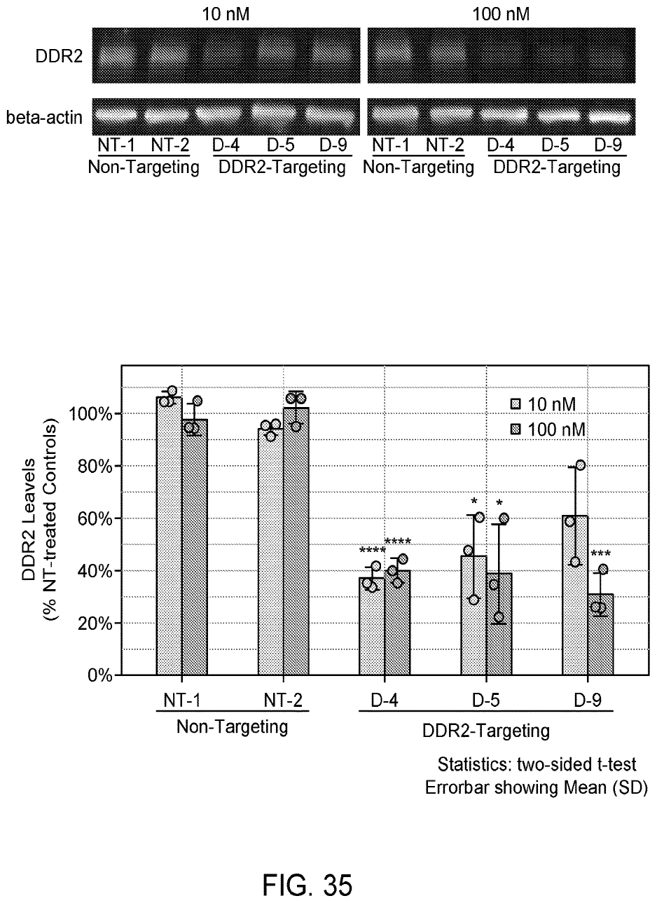

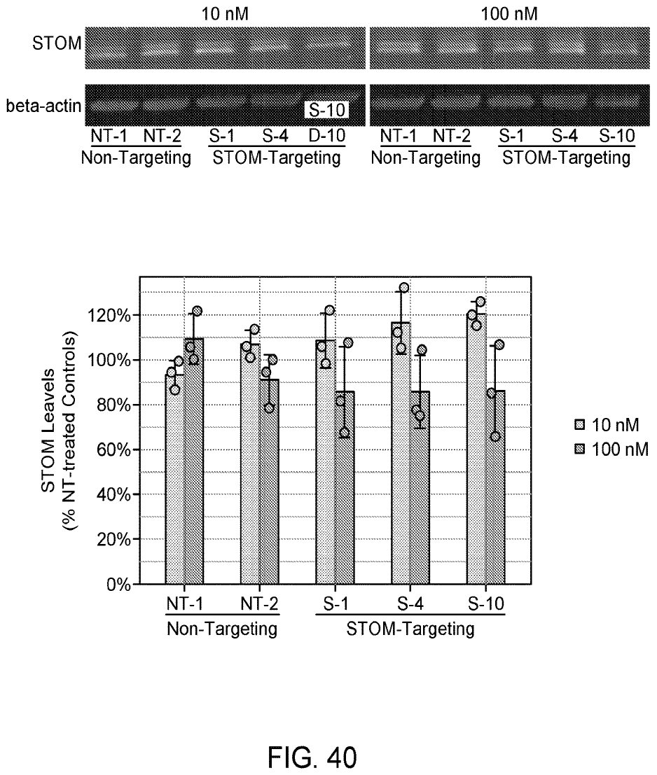

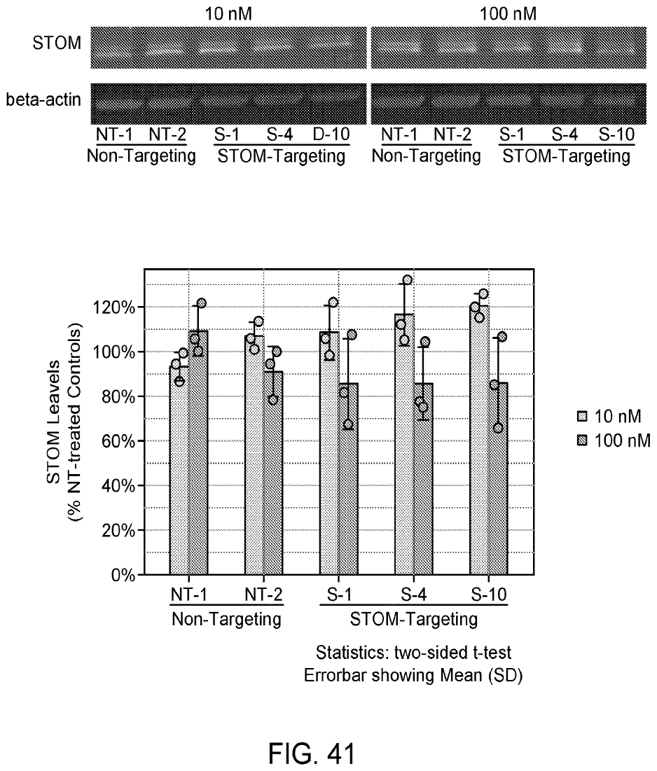

Dose-response curves for 8 different concentrations of ASOs in H4 cells are shown in FIG. 34. Knock-down was assessed by measuring polypeptide levels (FIGS. 35, 40, 41) using western Blot. These results demonstrate that ASOs resulted in significant gene knock down without inducing toxicity.

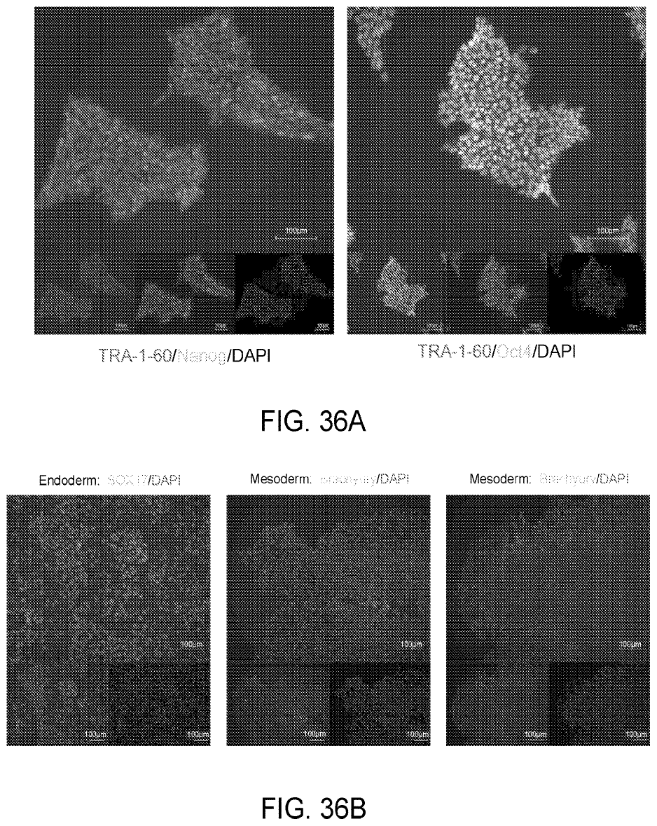

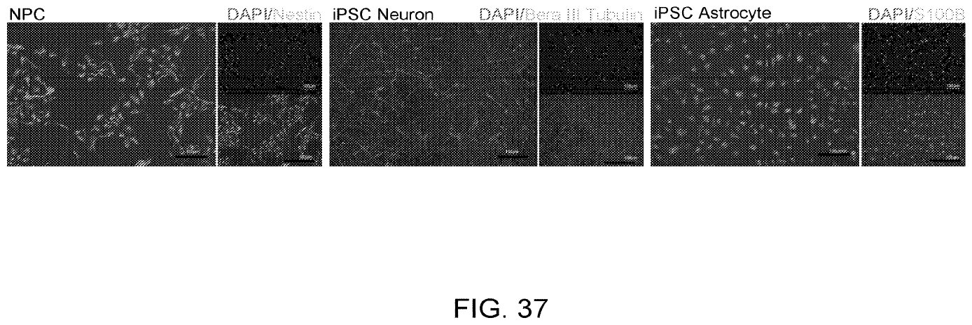

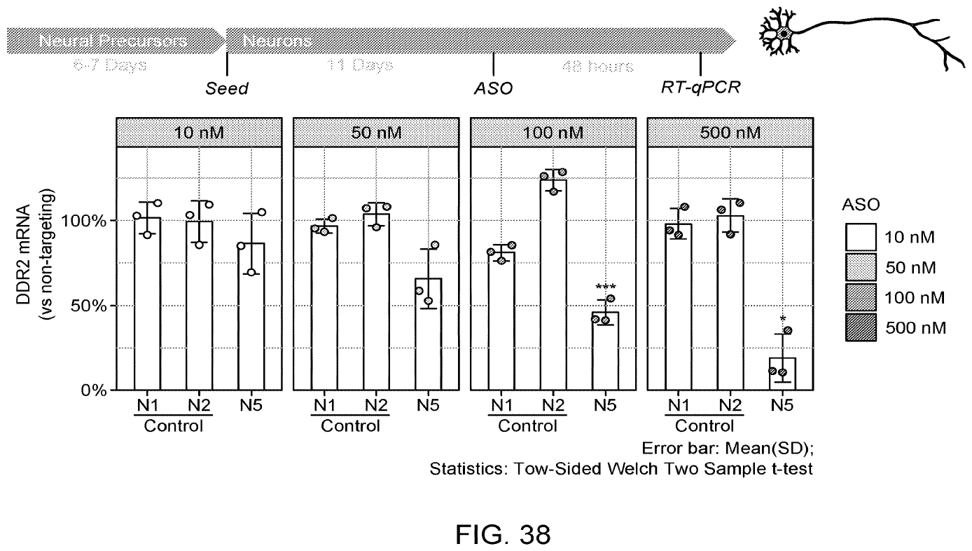

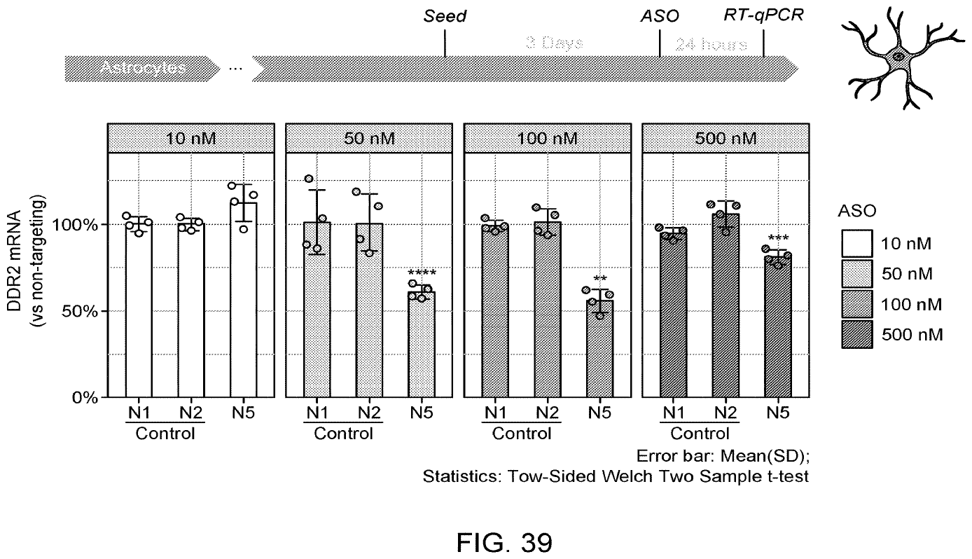

Induced pluripotent stem cells (iPSCs) were examined as a model to assess target engagement of the ASO D5 (targeting DDR2). It was confirmed that iPSCs expressed pluripotency markers (FIG. 36A) and could be differentiated into three germ layers (FIG. 36B), indicating that iPSCs could also be differentiated into neuronal progenitor cells (NPCs), which can then be differentiated into either neurons or astrocytes (FIG. 37). ASO D5 was administered to iPSCs-derived astrocytes and neurons separately following the schematic depicted in FIGS. 38-39. ASO D5 reduced DDR2 mRNA in both iPSC-derived neurons (FIG. 38) and iPSC-derived astrocytes (FIG. 39).

### Example 5: Treating Progressive Supranuclear Palsy

A human identified as having PSP is administered one or more inhibitors of a DDR2 polypeptide (e.g., nucleic acid molecules designed to induce RNAi of DDR2 polypeptide expression such as the nucleic acid molecules set forth in any one of SEQ ID NOs: 1-16) by intraperitoneal injection or intraabdominal injection. The administered inhibitor(s) can reduce or slow neurodegeneration (e.g., tau-related neurodegeneration) within the brain of the human and/or to reduce a level of tau polypeptides (e.g., 4-repeat tau polypeptides) in the brain of the human.

### Example 6: Treating Progressive Supranuclear Palsy

A human identified as having PSP is administered one or more inhibitors of a KANK2 polypeptide (e.g., nucleic acid molecules designed to induce RNAi of KANK2 polypeptide expression such as the nucleic acid molecules set forth in any one of SEQ ID NOs: 17-26) by intraperitoneal injection or intraabdominal injection. The administered inhibitor(s) can reduce or slow neurodegeneration (e.g., tau-related neurodegeneration) within the brain of the human and/or to reduce a level of tau polypeptides (e.g., 4-repeat tau polypeptides) in the brain of the human.

### Example 7: Treating Progressive Supranuclear Palsy

A human identified as having PSP is administered one or more inhibitors of a STOM polypeptide (e.g., nucleic acid molecules designed to induce RNAi of STOM polypeptide expression such as the nucleic acid molecules set forth in any one of SEQ ID NOs: 27-36) by intraperitoneal injection or intraabdominal injection. The administered inhibitor(s) can reduce or slow neurodegeneration (e.g., tau-related neurodegeneration) within the brain of the human and/or to reduce a level of tau polypeptides (e.g., 4-repeat tau polypeptides) in the brain of the human.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having progressive supranuclear palsy (PSP), wherein said method comprises administering an inhibitor of a glial polypeptide to said mammal.

2. The method of claim 1, wherein said mammal is a human.

3. The method of claim 1, wherein said method comprises identifying said mammal as having said PSP prior to said administering.

4. The method of claim 1, wherein said inhibitor of a glial polypeptide inhibits a discoidin domain receptor tyrosine kinase 2 (DDR2) polypeptide, a KN motif and ankyrin repeat domains 2 (KANK2) polypeptide, or a stomatin (STOM) polypeptide.

5-27. (canceled)

28. A method for treating a mammal having progressive supranuclear palsy (PSP), wherein said method comprises administering an inhibitor of a KN motif and ankyrin repeat domain 2 (KANK2) polypeptide to said mammal.

29. The method of claim 28, wherein said mammal is a human.

30-31. (canceled)

32. The method of claim 28, wherein said administering further comprises administering an inhibitor of a DDR2 polypeptide.

33-35. (canceled)

36. The method of claim 28, wherein said administering further comprises administering an inhibitor of a STOM polypeptide.

37-46. (canceled)
